SEQUENCE LISTING (56 sequences)

SEQ ID NO: 1 - protein, 475 aa, synthetic construct [sequence omitted]

SEQ ID NO: 2 - protein, 85 aa, synthetic construct
  REGION 1..85: note = RING domain of human TRIM21
MASAARLTMMWEEVTCPICLDPFVEPVSIECGHSFCQECISQVGKGGGSVCPVCRQRFLLKNLRPNRQLANMVNNLKEISQEARE

SEQ ID NO: 3 - protein, 79 aa, synthetic construct
  REGION 1..79: note = Amino acids 3-81 of human TRIM21
SAARLTMMWEEVTCPICLDPFVEPVSIECGHSFCQECISQVGKGGGSVCPVCRQRFLLKNLRPNRQLANMVNNLKEISQ

SEQ ID NO: 4 - protein, 81 aa, synthetic construct
  REGION 1..81: note = Amino acids 1-81 of human TRIM21
MASAARLTMMWEEVTCPICLDPFVEPVSIECGHSFCQECISQVGKGGGSVCPVCRQRFLLKNLRPNRQLANMVNNLKEISQ

SEQ ID NO: 5 - protein, 80 aa, synthetic construct
  REGION 1..80: note = Amino acids 2-81 of human TRIM21
ASAARLTMMWEEVTCPICLDPFVEPVSIECGHSFCQECISQVGKGGGSVCPVCRQRFLLKNLRPNRQLANMVNNLKEISQ

SEQ ID NO: 6 - protein, 12 aa, synthetic construct
  REGION 1..12: note = ALFAtag epitope
SRLEELRRRLTE

SEQ ID NO: 7 - protein, 52 aa, synthetic construct
  REGION 1..52: note = Amino acids 286-337 of human TRIM21
AVHITLDPDTANPWLILSEDRRQVRLGDTQQSIPGNEERFDSYPMVLGAQHF

SEQ ID NO: 8 - protein, 127 aa, synthetic construct
  REGION 1..127: note = Amino acids 339-465 of human TRIM21
SGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPL

SEQ ID NO: 9 - protein, 180 aa, synthetic construct
  REGION 1..180: note = PRYSPRY domain
AVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPL

SEQ ID NO: 10 - protein, 38 aa, synthetic construct
  REGION 1..38: note = B-box domain
RCAVHGERLHLFCEKDGKALCWVCAQSRKHRDHAMVPL

SEQ ID NO: 11 - protein, 110 aa, synthetic construct
  REGION 1..110: note = Coiled coil domain
EEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHS

SEQ ID NO: 12 - protein, 7 aa, synthetic construct
  REGION 1..7: note = Linker sequence
GSGGGGS

SEQ ID NO: 13 - protein, 15 aa, synthetic construct
  REGION 1..15: note = Linker sequence
GTQGERGLKKMLRTC

SEQ ID NO: 14 - protein, 4 aa, synthetic construct
  REGION 1..4: note = N-Acetyltransferase recognition site
DDDI

SEQ ID NO: 15 - protein, 4 aa, synthetic construct
  REGION 1..4: note = N-Acetyltransferase recognition site
EEEI

SEQ ID NO: 16 - protein, 6 aa, synthetic construct
  REGION 1..6: note = tev site
ENLYVQ

SEQ ID NO: 17 - protein, 438 aa, synthetic construct
  REGION 1..438: note = Human Caveolin-1 with c-terminal mEGFP
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEIPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLKEFVTAAGITLGMDELYKYSDLELKLRIL

SEQ ID NO: 18 - protein, 734 aa, synthetic construct
  REGION 1..734: note = Canine Caveolin-1 with c-terminal mEGFP and Halo tag
MSGGKYVDSEGHLYTVPIREQGNIYKPNNKAMAEEMSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTFCDPFFEAVGKIFSNIRINMQKETPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLKEFVTAAGITLGMDELYKYSDLEGGGGSMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 19 - protein, 486 aa, synthetic construct
  REGION 1..486: note = Human TRIM21 with c-terminal HA tag, driven by native TRIM21 promoter
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDYGGYPYDVPDYA

SEQ ID NO: 20 - protein, 350 aa, synthetic construct
  REGION 1..350: note = GFP nanobody fused to Fc region of human IgG1
MDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSLQMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 410 aa, synthetic construct
  REGION 1..410: note = TRIM21 RING, Box, Coiled-coil fused to GFP nanobody
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAGSGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 22 - protein, 209 aa, synthetic construct
  REGION 1..209: note = TRIM21 RING fused to GFP nanobody
MASAARLTMMWEEVTCPICLDPFVEPVSIECGHSFCQECISQVGKGGGSVCPVCRQRFLLKNLRPNRQLANMVNNLKEISQEAREGSGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 23 - protein, 369 aa, synthetic construct
  REGION 1..369: note = 12 copies of ALFAtag epitope n-terminal to GFP nanobody
MPSRLEEELRRRLTEPGGGGSPSRLEEELRRRLTEPGGGGSPSRLEEELRRRLTEPGGGGSPSRLEEELRRRLTEPGGGGSGSATMPSRLEEELRRRLTEPGGGGSPSRLEEELRRRLTEPGGGGSPSRLEEELRRRLTEPGGGGSPSRLEEELRRRLTEPGGGGSRIQATMPSRLEEELRRRLTEPGGGGSPSRLEEELRRRLTEPGGGGSPSRLEEELRRRLTEPGGGGSPSRLEEELRRRLTEPGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 24 - protein, 369 aa, synthetic construct
  REGION 1..369: note = 12 copies of ALFAtag epitope n-terminal to GFP nanobody (K45R, K67R, K89R)
MPSRLEEELRRRLTEPGGGGSPSRLEEELRRRLTEPGGGGSPSRLEEELRRRLTEPGGGGSPSRLEEELRRRLTEPGGGGSGSATMPSRLEEELRRRLTEPGGGGSPSRLEEELRRRLTEPGGGGSPSRLEEELRRRLTEPGGGGSPSRLEEELRRRLTEPGGGGSRIQATMPSRLEEELRRRLTEPGGGGSPSRLEEELRRRLTEPGGGGSPSRLEEELRRRLTEPGGGGSPSRLEEELRRRLTEPGGGGSMDQVQLVESGGALVQPGGSLRLSCAASGFPVNRYSMRWYRQAPGREREWVAGMSSAGDRSSYEDSVRGRFTISRDDARNTVYLQMNSLRPEDTAVYYCNVNVGFEYWGQGTQVTVSS

SEQ ID NO: 25 - protein, 224 aa, synthetic construct
  REGION 1..224: note = TRIM21 RING fused to ALFAtag nanobody with c-terminal HA eptiope
MASAARLTMMWEEVTCPICLDPFVEPVSIECGHSFCQECISQVGKGGGSVCPVCRQRFLLKNLRPNRQLANMVNNLKEISQEAREGSGGGGSEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGYPYDVPDYA

SEQ ID NO: 26 - protein, 224 aa, synthetic construct
  REGION 1..224: note = TRIM21 RING (K45R, K61R, K77R) fused to ALFAtag nanobody with c-terminal HA eptiope
MASAARLTMMWEEVTCPICLDPFVEPVSIECGHSFCQECISQVGRGGGSVCPVCRQRFLLRNLRPNRQLANMVNNLREISQEAREGSGGGGSEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGYPYDVPDYA

SEQ ID NO: 27 - protein, 224 aa, synthetic construct
  REGION 1..224: note = TRIM21 RING (K45R, K61R, K77R) fused to ALFAtag nanobody (K79R, K89R) with c-terminal HA eptiope (K79R, K89R) with c-terminal HA eptiope
MASAARLTMMWEEVTCPICLDPFVEPVSIECGHSFCQECISQVGRGGGSVCPVCRQRFLLRNLRPNRQLANMVNNLREISQEAREGSGGGGSEVQLQESGGGLVQPGGSLRLSCTASGVTISALNAMAMGWYRQAPGERRVMVAAVSERGNAMYRESVQGRFTVTRDFTNRMVSLQMDNLRPEDTAVYYCHVLEDRVDSFHDYWGQGTQVTVSSGYPYDVPDYA

SEQ ID NO: 28 - protein, 133 aa, synthetic construct
  REGION 1..133: note = ALFAtag nanobody with c-terminal HA eptiope
MEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGYPYDVPDYA

SEQ ID NO: 29 - protein, 133 aa, synthetic construct
  REGION 1..133: note = ALFAtag nanobody (K79R, K89R) with c-terminal HA eptiope
MEVQLQESGGGLVQPGGSLRLSCTASGVTISALNAMAMGWYRQAPGERRVMVAAVSERGNAMYRESVQGRFTVTRDFTNRMVSLQMDNLRPEDTAVYYCHVLEDRVDSFHDYWGQGTQVTVSSGYPYDVPDYA

SEQ ID NO: 30 - protein, 318 aa, synthetic construct
  REGION 1..318: note = TRIM21 RING with N-terminal GST-Tev
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDSSGSENLYFQGSHMASAARLTMMWEEVTCPICLDPFVEPVSIECGHSFCQECISQVGKGGGSVCPVCRQRFLLKNLRPNRQLANMVNNLKEISQEARE

SEQ ID NO: 31 - protein, 622 aa, synthetic construct
  REGION 1..622: note = TRIM21 RING fused to TRIM21 PRYSPRY with N-terminal GST-Tev-SUMO
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDSSGSENLYFQGSHMGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 32 - protein, 707 aa, synthetic construct
  REGION 1..707: note = Tandem TRIM21 RINGs fused to TRIM21 PRYSPRY with N-terminal GST-Tev-SUMO
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDSSGSENLYFQGSHMGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 33 - protein, 317 aa, synthetic construct
  REGION 1..317: note = TRIM21 RING fused GFP nanobody with N-terminal 6His-SUMO
MGSSHHHHHHGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMASAARLTMMWEEVTCPICLDPFVEPVSIECGHSFCQECISQVGKGGGSVCPVCRQRFLLKNLRPNRQLANMVNNLKEISQEAREGSGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 34 - protein, 317 aa, synthetic construct
  REGION 1..317: note = TRIM21 RING (K45R, K61R, K77R) fused GFP nanobody (K45R, K67R, K77R) with N-terminal 6His-SUMO
MGSSHHHHHHGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMASAARLTMMWEEVTCPICLDPFVEPVSIECGHSFCQECISQVGRGGGSVCPVCRQRFLLRNLRPNRQLANMVNNLREISQEAREGSGGGGSMDQVQLVESGGALVQPGGSLRLSCAASGFPVNRYSMRWYRQAPGREREWVAGMSSAGDRSSYEDSVRGRFTISRDDARNTVYLQMNSLRPEDTAVYYCNVNVGFEYWGQGTQVTVSS

SEQ ID NO: 35 - protein, 316 aa, synthetic construct
  REGION 1..316: note = TRIM21 RING (I18R/M72E) fused GFP nanobody with N-terminal 6His-SUMO
MAHHHHHHMGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMASAARLTMMWEEVTCPRCLDPFVEPVSIECGHSFCQECISQVGKGGGSVCPVCRQRFLLKNLRPNRQLANEVNNLKEIRQEAREGSGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 36 - protein, 76 aa, synthetic construct [sequence omitted]

SEQ ID NO: 37 - protein, 1291 aa, synthetic construct
  REGION 1..1291: note = Ube1 with N-terminal GST-Tev
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDSSGSENLYFQGSHMSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 38 - protein, 166 aa, synthetic construct
  REGION 1..166: note = Ube2N with N-terminal 6His-Tev
MAHHHHHENLYFQGMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 39 - protein, 171 aa, synthetic construct
  REGION 1..171: note = Ube2N (D119A) with N-terminal 6His-Tev
MAHHHHHHSSGSENLYFQGMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDAPLANDVAEQWKTNEAQAIETARAWTRLYAMNNI

SEQ ID NO: 40 - protein, 380 aa, synthetic construct
  REGION 1..380: note = Ube2V2 with N-trminal GST-Tev
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDSSGSENLYFQGSQEFMAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 41 - protein, 275 aa, synthetic construct
  REGION 1..275: note = mEGFP with N-terminal 6His
MGSSHHHHHHSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLKEFVTAAGITLGMDELYKYSDLELKLRILQSTARDPVQVKRPL

SEQ ID NO: 42 - protein, 216 aa, synthetic construct
  REGION 1..216: note = Chaetomium thermophilum Naa50 residues 82-289 with C-terminal 6His
MGLPSGASIVPLAQEHIPALRRINSLLLPVAYPDSFYHKALDPLASGLFSRAILWQDTNADPPKVVGGLICRLEPNPFLSVTGEPTPVQLPADQPQRAPQAPKDTPFHAIYIQSLALLSPYRSLGLAAAALDHIIATAAVLPAAGSNIDARTIYAHVWTENEEGLKWYESRGFVKEGGEPVKGYYFKLRPDTAWIVRRHIGESAKLNDVVHHHHHH

SEQ ID NO: 43 - protein, 88 aa, synthetic construct
  REGION 1..88: note = TRIM21 RING (N-terminal GSH scar)
GSHMASAARLTMMWEEVTCPICLDPFVEPVSIECGHSFCQECISQVGKGGGSVCPVCRQRFLLKNLRPNRQLANMVNNLKEISQEARE

SEQ ID NO: 44 - protein, 290 aa, synthetic construct
  REGION 1..290: note = TRIM21 RING fused to TRIM21 PRYSPRY (native N-terminus)
MASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 45 - protein, 375 aa, synthetic construct
  REGION 1..375: note = Tandem TRIM21 RINGs fused to TRIM21 PRYSPRY (native N-terminus)
MASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 46 - protein, 209 aa, synthetic construct; identical to SEQ ID NO: 22
  REGION 1..209: note = TRIM21 RING fused to GFP nanobody (native N-terminus)

SEQ ID NO: 47 - protein, 209 aa, synthetic construct
  REGION 1..209: note = TRIM21 RING (K45R, K61R, K77R) fused GFP nanobody (K45R, K67R, K77R) (native N-terminus)
MASAARLTMMWEEVTCPICLDPFVEPVSIECGHSFCQECISQVGRGGGSVCPVCRQRFLLRNLRPNRQLANMVNNLREISQEAREGSGGGGSMDQVQLVESGGALVQPGGSLRLSCAASGFPVNRYSMRWYRQAPGREREWVAGMSSAGDRSSYEDSVRGRFTISRDDARNTVYLQMNSLRPEDTAVYYCNVNVGFEYWGQGTQVTVSS

SEQ ID NO: 48 - protein, 209 aa, synthetic construct
  REGION 1..209: note = TRIM21 RING (I18R/M72E) fused GFP nanobody (native N-terminus)
MASAARLTMMWEEVTCPRCLDPFVEPVSIECGHSFCQECISQVGKGGGSVCPVCRQRFLLKNLRPNRQLANEVNNLKEIRQEAREGSGGGGSMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 49 - protein, 76 aa, synthetic construct [sequence omitted]

SEQ ID NO: 50 - protein, 1291 aa, synthetic construct; identical to SEQ ID NO: 37
  REGION 1..1291: note = Ube1 with N-terminal GST

SEQ ID NO: 51 - protein, 153 aa, synthetic construct
  REGION 1..153: note = Ube2N (N-terminal G scar)
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 52 - protein, 153 aa, synthetic construct
  REGION 1..153: note = Ube2N (D119A) (N-terminal G scar)
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDAPLANDVAEQWKTNEAQAIETARAWTRLYAMNNI

SEQ ID NO: 53 - protein, 150 aa, synthetic construct
  REGION 1..150: note = Ube2V2 (N-terminal GSQEF scar)
GSQEFMAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 54 - protein, 275 aa, synthetic construct; identical to SEQ ID NO: 41
  REGION 1..275: note = mEGFP with N-terminal 6His

SEQ ID NO: 55 - protein, 216 aa, synthetic construct; identical to SEQ ID NO: 42
  REGION 1..216: note = Chaetomium thermophilum Naa50 residues 82-289 with C-terminal 6His

SEQ ID NO: 56 - protein, 9 aa, synthetic construct
  REGION 1..9: note = HA epitope
YPYDVPDYA

(19) United States
(12) Patent Application Publication
(10) Pub. No.: US 2025/0376495 A1
(43) Pub. Date: Dec. 11, 2025

(54) MODIFIED FUSION PROTEINS AND NUCLEIC ACID CONSTRUCTS

(71) Applicant: UNITED KINGDOM RESEARCH AND INNOBATION, Swindon, Wiltshire (GB)

(72) Inventors: Leo JAMES, Swindon (GB); Leo KISS, Swindon (GB); Dean CLIFT, Swindon (GB); Tyler RHINESMITH, Swindon (GB)

(21) Appl. No.: 18/877,919

(22) PCT Filed: Jun. 28, 2023
(86) PCT No.: PCT/EP2023/067745

§ 371 Date: Dec. 20, 2024

(30) Foreign Application Priority Data
Jun. 29, 2022 (GB) 2209517.8

(51) Int. Cl. C07K 14/47; C12N 15/62; C12N 15/86

(52) U.S. Cl. CPC C07K 14/4702; C12N 15/62; C12N 15/86; C07K 2319/915; C12N 2750/14143

(57) ABSTRACT

The present invention relates to modified fusion proteins and nucleic acid constructs suitable for use for protein degradation in cells. The fusion proteins comprise a RING domain; and an adaptor domain that is capable of localising the RING domain with a substrate. The fusion proteins are unable to undergo N-terminal autoubiquitination and have increased cellular half-life. The present invention also relates to compositions comprising these fusion proteins and nucleic acids, and the use of the fusion proteins and nucleic acid constructs in therapy.

### FIELD OF THE INVENTION

The present invention relates to modified fusion proteins and nucleic acid constructs suitable for use for protein degradation in cells. The fusion proteins are unable to undergo N-terminal autoubiquitination and have increased cellular half-life. The present invention also relates to compositions comprising these fusion proteins and nucleic acids, and the use of the fusion proteins and nucleic acid constructs in therapy.

### BACKGROUND OF THE INVENTION

Protein degradation occurs naturally within cells and provides an endogenous mechanism to prevent the occurrence of misfolded proteins, and to mediate cellular responses. The major pathway for protein degradation is via the ubiquitin-proteasome system (UPS). The ability to manipulate the UPS in order to redirect the system and provide targeted protein degradation within cells has enormous potential for applications in research, drug discovery and therapeutics.

Selective depletion of a target protein enables the study of protein function and dynamic protein interactions at the cellular level. Such selective depletion is of particular use in drug discovery, where small molecules known as “proteolysis-targeting chimeras” (PROTACs) can be used to redirect protein degradation to induce selective depletion of a target protein (Schapira et al, 2019). Similarly, technologies such as “Trim-Away™” utilise a specific component of the UPS, an E3 ubiquitin ligase known as TRIM21, to selectively deplete antibody-bound target proteins (Clift et al. 2017; Zeng et al (2021); Castro-Dopico, T., et al. 2019; Chen, X et al. 2019). A further strategy utilising constructs comprising RING domains of E3 ubiquitin ligases is also disclosed in WO2022175549. These emerging tools and drug discovery platforms enable the study of protein interactions in a post-translational setting, and avoid many limitations associated with genetic manipulation, which can fail to provide phenotypic insight and can be costly and time-consuming.

Targeted protein degradation holds potential for use in therapeutic applications (Wu, T, et al. 2020) in particular for use in diseases associated with excessive protein production or aberrant protein aggregation. The use of targeted protein degradation as a therapeutic strategy could minimise the off-target effects of drugs and avoid or reduce systemic drug exposure.

TRIM proteins are the largest family of E3 ligases in mammals. They include TRIMs that suppress viral infection (TRIM5 (Stremlau et al., 2004), TRIM21 (Mallery et al., 2010), TRIM22 (Pagani et al., 2021), TRIM25 (Galao et al., 2022)), activate innate immunity (TRIM32 (Zhang et al., 2012), TRIM56 (Tsuchida et al., 2010), TRIM65 (Kato et al., 2021), RIPLET (Cadena et al., 2019)), and repress transcription (TRIM4 (Herquel et al., 2011), TRIM28 (Robbez-Masson et al., 2018)). In particular the intracellular antibody receptor TRIM21, is responsible for mediating targeted protein degradation during Trim-Away™. Despite their importance, the ubiquitination mechanism of TRIM ligases has remained elusive. TRIM ligases contain both substrate-targeting and catalytic domains in one polyprotein. However, how TRIMs catalyse ubiquitination is incompletely understood, particularly in terms of activation, ubiquitin priming and chain extension.

Current mechanisms of TRIM catalysis have been informed primarily by experiments on the two antiviral proteins TRIM5 and TRIM21. Both proteins are dimers containing a RING, B Box, coiled-coil and PRYSPRY domains. Each RING domain is arranged at opposite ends of the elongated antiparallel coiled-coil (Sanchez et al., 2014b) and whilst ubiquitination of monomeric RINGs can be detected in vitro, dimerization is required for full cellular activity (Dickson et al., 2018; Zeng et al., 2021). TRIM21 also undergoes supramolecular clustering (Zeng et al., 2021), including on the surface of viral capsids (McEwan et al., 2012), but is anchored to its substrates by an intermediate antibody molecule (Mallery et al., 2010): The Fabs of each antibody bind the substrate whilst the Fc is bound by the TRIM21 PRYSPRY (James et al., 2007).

TRIM ligases undergoes degradation along with its substrate. This has been shown for TRIM5 during HIV infection (Rold and Aiken, 2008) and for TRIM21 with a wide-range of substrates during Trim-Away (Clift et al., 2017). Moreover, TRIM21 and its substrates are degraded with matching kinetics suggesting that they are processed together as a complex (Clift et al., 2017). In support of TRIM ligase self-degradation, light-induced clustering of a TRIM21 RING-crytochrome2 fusion was sufficient to cause ligase degradation, (Zeng et al., 2021). Meanwhile, TRIM5 self-degradation can be induced simply by ectopic overexpression (Diaz-Griffero et al., 2006), which leads to the formation of large oligomers called ‘cytoplasmic bodies’, likely driven by B Box trimerization (Diaz-Griffero et al., 2009; Wagner et al., 2016).

It has been proposed that ligase autoubiquitination alone may drive proteasome recruitment, resulting in degradation of the entire TRIM:substrate complex (Diaz-Griffero et al., 2006; Kiss and James, 2022; Mallery et al., 2010; Towers, 2007). Due to the degradation of the entire TRIM:substrate complex intracellular turnover of the TRIM containing proteins can be high.

Consequently, there is a need for further fusion proteins and corresponding nucleic acid constructs which can be used to selectively degrade proteins in cells which would have an improved cellular half-life. Such fusion proteins would be useful in particular in both therapeutic and research settings.

### SUMMARY OF THE INVENTION

The present invention is directed to fusion proteins that do not undergo N-terminal autoubiquitination and nucleic acid constructs that encode such proteins, suitable for degrading proteins in cells. Specifically, fusion proteins that comprise at least one RING domain and an adaptor sequence, suitable for degrading proteins in cells. The inventors have surprisingly found that modification of the N-terminal of the fusion protein uncouples ligase and substrate degradation. The inventors have provided fusion proteins that can be used to degrade protein wherein the target substrate is degraded, whilst the fusion protein is not.

In a first aspect, the present invention provides a fusion protein comprising:
at least a first RING domain; and
an adaptor domain that is capable of localising the RING domain with a substrate, and
   wherein the fusion protein is incapable of N-terminal autoubiquitination.

The fusion protein comprising the RING domain is unable to undergo N-terminal autoubiquitination. The fusion protein is not able to act as a substrate for the E2 enzyme Ube2W. The N-terminus of the fusion protein is modified to inhibit ubiquitination of the fusion protein itself by E2 enzymes, in particular Ube2W. The E2 enzyme Ube2W is still capable of binding to the fusion protein. The fusion protein can use Ube2W to ubiquitinate substrates of the fusion protein, but the fusion protein is inhibited from N-terminally ubiquitinating itself (e.g. autoubiquitination). The inventors have found that inhibiting the ability of RING comprising fusion proteins to undergo N-terminal autoubiquitination slows self-turnover of the fusion proteins, i.e. increases cellular half-life, allowing them to persist in cells for longer without preventing substrate degradation. The fusion proteins are not degraded alongside their target substrate. The modified fusion proteins have an increased half-life in cells as compared to an equivalent unmodified fusion protein, whilst still maintaining the ability to degrade the substrate protein in cells.

A second aspect of the invention provides a nucleic acid construct encoding the fusion protein according to the first aspect.

A third aspect of the invention provides a nucleic acid construct comprising a first nucleic acid sequence encoding a first RING domain, and a second nucleic acid sequence encoding an adaptor domain, wherein the nucleic acid construct encodes for a fusion protein that is incapable of N-terminal autoubiquitination.

A fourth aspect of the invention provides a pharmaceutical composition comprising a fusion protein according to the first aspect or a nucleic acid according to the second aspect, and a pharmaceutically acceptable carrier and/or excipient.

A fifth aspect of the invention provides a fusion protein according to the first aspect or a nucleic construct according to the second aspect for use as a medicament.

A sixth aspect of the invention provides a method of degrading a target protein in a cell comprising introducing a fusion protein according to the first aspect or a nucleic construct according to the second aspect into the cell.

A seventh aspect of the invention provides a method of increasing the cellular half-life of a fusion protein comprising a RING domain and an adaptor domain where the adaptor domain is capable of localising the RING domain with a substrate, the method comprising modifying the fusion protein such that it is incapable of N-terminal autoubiquitination.

An eighth aspect of the invention a provides a method of producing the fusion protein according to the first aspect, comprising;
(a) culturing a host cell comprising a first vector encoding for a fusion protein comprising a RING domain and an adaptor domain, under conditions to permit expression of the fusion protein; and
(b) obtaining the expressed protein from the host cell, wherein the fusion protein is incapable of N-terminal autoubiquitination.

Further aspects and embodiments of the invention are described below.

### BRIEF DESCRIPTION OF FIGURES

FIG. 1: Dimeric Ube2W and RING clustering is required for ligase autoubiquitination (A) 2.25 Å X-ray structure of TRIM21 RING (blue) in complex with Ube2W (pink). (B) Close-up of the E2:E3 interface. (C) Structural model of a RING:Ube2W˜Ub complex based on superposition of the RING:Ube2W structure and the Ub-RING:Ube2N-Ub:Ube2V2 structure (7BBD)(Kiss et al., 2021). Ube2N-Ub was superposed onto Ube2W. (D) Close-up of the RING:Ube2W˜Ub model showing a potential salt bridge between TRIM21 E13 and ubiquitin K11. (E) Schematic model of the catalytic RING topology for N-ubiquitination of TRIM21 by a Ube2W dimer. (F) Ube2W-mediated TRIM21 RING (R) mono-ubiquitination assay using 10 μM T21-R-PS or R-R-PS and 0.25 μM Ube2W WT or monomeric V30K/D67K. Representative example of n=2 independent experiments. (G) Schematic model of antibody-induced recruitment of either two RINGs or two RING dimers. Only the latter satisfies the ‘two-plus-one’ model for RING autoubiquitination (Fletcher et al., 2018; Kiss et al., 2021). (H) Antibody induced N-ubiquitination of 100 nM T21-R-PS or R-R-PS in the absence or presence of 1 molar equivalent of anti-GFP antibody. Ube2W was titrated (25, 50, 100, 200 nM). Representative example from n=3 independent experiments. (1) Quantification of monoubiquitination from (H). Graph shows mean and s.e.m. from n=3 independent experiments. (J) RPE-1 CAV1-mEGFP cells were electroporated with PBS or anti-GFP antibody±indicated E2 proteins and 24 h later CAV1-mEGFP fluorescence was quantified using the IncuCyte system. Values normalized to PBS control condition. Graph shows mean and s.e.m from n=3 independent experiments. Statistical significance in (I) and (J) is based on two-tailed Student's t-test.

FIG. 2: Ligase N-terminal ubiquitination is not required for substrate degradation and antiviral activity. (A) Schematic model showing N-terminal acetylation of TRIM21 by AcCoA and NAT and then incubation of the acetylated or unmodified ligase with ubiquitin (Ub) and Ube2W in a ubiquitination reaction. (B) Protein-stained gel of ubiquitination reaction depicted in (A) using TRIM21 R-R-PS ligase. Monoubiquitination of ligase that has been incubated with AcCoA and NAT is inhibited. Representative example from n=3 independent experiments. (C) Schematic model showing Trim-Away experiment in which antibody is electroporated into cells together with R-R-PS (±N-acetylation). Once inside cells, a ternary complex with the target protein is formed. If degradation is driven by ligase N-terminal ubiquitination, then N-terminally acetylated R-R-PS activity will be inhibited. (D) Results of Trim-Away experiment described in (C) shows that N-terminal acetylation of the ligase does not alter the kinetics of substrate (CAV1-GFP) degradation. RPE-1 CAV1-mEGFP cells were electroporated with PBS or anti-GFP antibody (1.67 μM)±R-R-PS proteins (2.1 μM) and CAV1-mEGFP fluorescence was quantified using the IncuCyte system. Time shows hours (h) post-electroporation. Values normalized to PBS control condition. Graphs shows mean and s.e.m. from n=4 technical replicates. Representative example from n=2 independent experiments. Note that there is CAV1-mEGFP degradation with anti-GFP alone due to the presence of endogenous cellular TRIM21. (E) Schematic model showing electroporation of N-acetylated ligase (Ac-R-R-PS) into cells followed by infection with Adv5 in the presence of anti-hexon antibody 9C12. If ligase N-terminal ubiquitination is necessary for TRIM21 antiviral function, neutralization of infection and immune signaling will be inhibited. (F, G) Neutralization of AdV5 infection by increasing 9C12 concentrations (F) and AdV5-9C12-induced NFkB activation (G) in TRIM21 KO cells infected immediately after electroporation with PBS or R-R-PS±N-acetylation. 9C12H433A does not bind TRIM21 PRYSPRY. Graphs shows mean and s.e.m. from n=3 independent experiments. Statistical significance is based on two-way ANOVA (F) and Student's t-test (G).

FIG. 3: N-terminal ubiquitination regulates ligase turnover in cells. (A) Schematic model showing electroporation of R-R-PS±N-acetylation and ubiquitin-proteasome dependent self-degradation. (B) Western blot of experiment depicted in (A) 1 hour post-electroporation. R-R-PS protein levels are rescued by addition of 10 μM proteasome inhibitor epoxomycin. Acetylated R-R-PS protein persists in cells irrespective of proteasome inhibition. Representative example from n=2 independent experiments. (C) Schematic model showing electroporation of R-R-PS±N-acetylation into cells, followed by delayed Trim-Away or Adv5 neutralization assays. (D) For the delayed Trim-Away assay, mRNA (80 nM) encoding the antibody construct (vhhGFP4-Fc) responsible for recruiting R-R-PS to substrate (CAV1-mEGFP) is co-electroporated into NIH3T3-CAV1-mEGFP cells with PBS or R-R-PS±N-acetylation (2.4 μM); Trim-away is delayed for ˜2 h until vhhGFP4-Fc protein is translated. Graph shows mean and s.e.m. from 4 technical replicates of CAV1-mEGFP fluorescence quantified using the IncuCyte system. Values normalised to PBS control condition (no vhhGFP4-Fc). Time shows hours (h) post-electroporation Representative example from n=2 independent experiments. Note that NIH3T3 cells do not contain endogenous TRIM21 and expression of vhhGFP4-Fc in the absence of TRIM21 activity leads to GFP stabilization. (E) For the delayed Adv5 neutralisation assay, HEK293T TRIM21 KO cells are infected with AdV5±9C12 two hours post-electroporation of PBS or R-R-PS±N-acetylation. Graph shows mean and s.e.m. from n=3 independent experiments. Statistical significance is based on two-way ANOVA.

FIG. 4: TRIM21 independently ubiquitinates itself and its substrate. (A) Schematic showing an in vitro ubiquitination reaction in which ligase (R-R-PS), substrate (GFP) and anti-GFP antibody are incubated together with various E2 enzymes to promote either mono- or polyubiquitination. (B) Western blot of experiment described in (A). Top panel is blotted for GFP, middle panel for IgG and lower panel for TRIM21. Depending on the E2s present, monoubiquitinated species or a higher molecular weight smear indicative of polyubiquitin is observed. Representative example from n=2 independent experiments. (C) Western blot of experiment similar to (B) but comparing R-R-PS to Ac-R-R-PS. Note that while R-R-PS ubiquitinates itself, antibody heavy chain and substrate, Ac-R-R-PS only ubiquitinates antibody and substrate. Representative example from n=2 independent performed experiments. See also See also FIG. 9.

FIG. 5: Substrate ubiquitination parallels substrate degradation during Trim-Away. (A) Scheme showing Trim-Away experiment in which antibodies are electroporated into cells expressing endogenous TRIM21. Ubiquitination and degradation are then monitored in the presence or absence of proteasome inhibitor MG132. (B, C) RPE-1 cells were electroporated with PBS or (B) anti-ERK1 antibody or (C) anti-IKKα antibody and whole cell lysates harvested at the indicated times after electroporation for immunoblotting. Short exposures show degradation of TRIM21 and substrates. Long exposures reveal substrate ubiquitination followed by degradation of ubiquitinated species. (D) RPE-1 cells were electroporated with PBS or anti-IKKα antibody±MG132 and whole cell lysates harvested 3 h post-electroporation for immunoblotting. (E) RPE-1 TRIM21 KO cells reconstituted with TRIM21-HA or empty vector (EV) were electroporated with PBS or anti-ERK1 antibody±MG132 and whole cell lysates harvested 1 h post-electroporation for immunoblotting. Representative examples from 3 independent experiments.

FIG. 6: Trim-Away mediates protein depletion in the absence of lysine ubiquitination on either ligase or substrate. (A). Schematic showing T21R-vhhGFP4 fusion protein and substitutions to remove all lysines. (B, C) RPE-1 CAV1-mEGFP-Halo cells were electroporated with PBS or T21R-vhhGFP4 protein±lysines±N-acetylation. (B) CAV1-mEGFP-Halo fluorescence was quantified using the IncuCyte system. Time shows hours (h) post-electroporation. Values normalized to PBS control condition. Graphs shows mean and s.e.m. from n=4 technical replicates. (C) Whole cell lysates were harvested 3 h post-electroporation for immunoblotting. Representative examples (B, C) from 3 independent experiments. (D) Schematic showing a model lysine-less substrate consisting of a vhhGFP4 nanobody (Lysine substitutions in box) with 12 copies of the naturally lysine-less ALFAtag epitope. (E) Scheme showing Trim-Away experiment in which the anti-ALFAtag antibody (NbALFA-Fc) is electroporated into cells expressing endogenous TRIM21 and the lysine-less substrate (12×ALFAtag-vhhGFP4^{K0}). (F-H) RPE-1 WT or TRIM21 KO cells expressing either substrate with lysines (12×ALFAtag-vhhGFP4) or without lysines (12×ALFAtag-vhhGFP4^{K0}) were electroporated with PBS or NbALFA-Fc and whole cell lysates harvested 8 h post-electroporation for capillary-based immunoblotting. Lane view (F) and quantification (G, H) of substrate (G) and TRIM21 (H) protein levels normalized to PBS condition. Graphs show mean and s.e.m. from 2 independent experiments. Statistical significance is based on two-way ANOVA. Note that binding of NbALFA-Fc in the absence of TRIM21 causes stabilization of substrate. (1) Schematic of lysine-less Trim-Away assay. The substrate constitutes a vhhGFP4 nanobody with 12 copies of the ALFAtag epitope. The ligase constitutes the T21 RING fused to an anti-ALFAtag nanobody. The 12×ALFAtag allows clustering of multiple T21 RINGs, triggering degradation. The substitutions necessary to remove lysines from each domain in the assay are shown boxed. Note that the ALFAtag and HA epitopes are naturally lysine-less. (J, K) RPE-1 TRIM21 KO cells expressing either substrate with lysines (12×ALFAtag-vhhGFP4) or without lysines (12×ALFAtag-vhhGFP4^{KO}) were electroporated with water (control) or mRNA encoding the indicated constructs and whole cell lysates harvested 8 h post-electroporation for capillary-based immunoblotting. Lane view (J) and quantification (K) of substrate protein levels normalized to control condition. Graph shows mean and s.e.m. from 2 independent experiments. Statistical significance from control condition is based on two-way ANOVA. Note that binding of NbALFA alone causes stabilization of substrate.

FIG. 7: Interaction of Ube2W with TRIM21 RING. (A) Histograms of chemical shift perturbations (CSP) shown against the sequence of TRIM21 RING^{M10E} (R^{M10E}). These CSPs result from NMR titrations of Ube2W^{V30K/D67K/C91K} against ¹⁵N-labelled TRIM21 tri-ionic mutants at a 1:1 molar ratio. Blue circles indicate proline residues, white circles missing assignments. (B) A part of ¹⁵N-HSQC spectral overlay of R^{M10E} in absence (blue) or presence of 1:1 molar equivalent of Ube2W^{V30K/D67/C91K}. In addition, spectra of TRIM21 mutants (E12A in light green, E12R in dark green and E13A in orange) are shown in presence of 1:1 molar equivalent of Ube2W^{V30K/D67/C91K}. (C) Histograms shown in (A) are here shown as an overlay. (D) Ube2W-mediated TRIM21 RING monoubiquitination assay. Shown is a time-course, where error bars represent s.e.m. from n=3 independent experiments. Western blots are representative of all replicates. (E) Shown are RING dimers of different TRIM21 complexes (RING:Ube2W, RING-Box (5OLM)(Dickson et al., 2018), RING:Ube2N-Ub (two RING dimers in asymmetric unit, 6S53) (Kiss et al., 2019), Ub-RING:Ube2N-Ub:Ube2V2 (7BBD)(Kiss et al., 2021)). Zn²⁺-atoms are shown as grey spheres, the isopeptide bond is marked by an arrow and polar interactions are indicated by dashed black lines.

FIG. 8: TRIM21 RING can be N-terminally acetylated with NAT and AcCoA to block N-terminal autoubiquitination. (A) LC-MS/MS spectra of TRIM21 R-R-PS after 4 h acetylation reaction show N-acetylated TRIM21 N-terminal peptides after digestion with the protease N-Asp. (B) Instant-Blue-stained gels showing Ube2W-mediated TRIM21 mono-ubiquitination reactions with R-R-PS. Before the ubiquitination reaction, acetylation reactions were performed for the indicated times. Gel representative of n=2 independent experiments. (C) As in (B) but showing results of ubiquitination reaction upon incubation with Ube2W and Ube2N/V2 for 1 h after 4 h of N-acetylation. In contrast to N-terminal monoubiquitination, incubation with NAT and AcCoA doesn't impact ubiquitin smearing characteristic of free K63-chain formation. Gel representative of n=2 independent experiments.

FIG. 9: Ligase and substrate ubiquitination in vitro. (A) Western blots of ubiquitination reactions using combinations of R-R-PS, anti-GFP antibody, GFP, Ube2W and Ube2N/Ube2V2. Substrate and antibody heavy chain are only ubiquitinated in the presence of R-R-PS and substrate only in the presence of both antibody and R-R-PS. Western blots representative of n=2 independently performed experiments.

FIG. 10: Substrate ubiquitination during Trim-Away in live cells. (A) Western blots of Trim-Away time-course experiment using anti-IKKα antibody in the presence or absence of MG132. Proteasome inhibition rescues IKKα degradation and leads to the accumulation of ubiquitinated protein. Western blots representative of n=2 independent experiments. (B) Ubiquitination of CAV1-mEGFP by T21RBCC-vhhGFP4 or T21R-vhhGFP4 fusions in the presence or absence of MG132. Western blots representative of n=2 independent experiments. Ponceau protein stain shows equal loading. (C) Scheme showing electroporation of T21R-vhhGFP4 protein fusion into cells expressing CAV1-mEGFP. Clustering of the RING fusion may lead to ubiquitination on either substrate or ligase and complex degradation. (E) Western blot of Trim-Away experiment using WT T21R-vhhGFP4 or a mutant incapable of catalysing ubiquitination (T21R-vhhGFP4^{I18R/M72E}). Western blots representative of n=3 independent experiments.

FIG. 11: List of plasmid and sequences thereof (where appropriate) used in the examples.

FIG. 12: List of proteins and sequences thereof (where appropriate) used in the examples.

### DETAILED DESCRIPTION OF INVENTION

The inventors have found that inhibiting N-terminal autoubiquitination by the E2 enzyme Ube2W of a fusion protein comprising at least one RING domain and an adaptor domain increases the cellular half-life of the fusion protein whilst still maintaining its cellular activity.

Accordingly, the invention provides a fusion protein comprising:
at least a first RING domain; and
an adaptor sequence that is capable of localising the RING domain with a substrate, and
wherein the fusion protein is incapable of N-terminal autoubiquitination.

The fusion proteins of the invention have E3 ubiquitin ligase activity, however they are incapable of N-terminal autoubiquitination. In some embodiment the fusion proteins are incapable of being ubiquitinated. The fusion protein is capable of binding to the E2 enzyme Ube2W and using it to ubiquitinate substrates but is modified to prevent it from ubiquitinating itself (autoubiquitination). By being “incapable of N-terminal autoubiquitination”, “unable to undergo N-terminal autoubiquitination”, is “inhibited from being N-terminally autoubiquitinated”, or is “unable to be N-terminally autoubiquitinated” or similar, it means the fusion protein cannot autoubiquitinate itself but is capable of ubiquitinating other proteins present. In other words, the invention provides RING containing fusion proteins that are incapable of N-terminally ubiquitinating themselves but are still catalytically active and able to N-terminally ubiquitinate other proteins.

A RING has to be active to mediate target substrate degradation, however an active RING will also degrade itself unless its autoubiquitination is blocked. By blocking the N-terminus of the fusion protein it is possible to extend the half-life of the fusion proteins in cells. The inventors have found it is possible to block a RING-containing fusion protein's autoubiquitination without affecting its ability to mediate substrate degradation. Modifying the N-terminal of the fusion protein to inhibit autoubiquitination of the fusion protein means it will survive for longer periods once delivered into cells, thereby persisting long enough to degrade multiple copies of the substrate. The fusion proteins are no longer degraded alongside their target, thereby resulting in a more efficient and longer-lasting protein depletion.

Even in the absence of a target substrate, the RING can have some residual activity and can be a targeted by other ligases. Therefore, blocking the N-terminus makes RING containing fusion proteins more persistent in the cell.

The N-terminal of the fusion protein is modified in order to inhibit autoubiquitination of the fusion protein. Modification of the N-terminal of the fusion protein prevents N-terminal ubiquitination of the fusion protein by E2 enzymes for example Ube2W. This is accomplished by rendering the reactive N-terminus of the fusion protein incapable of being covalently modified with ubiquitin by E2 enzymes e.g. Ube2W. The fusion protein is still capable of binding Ube2W. The E2 enzymes, in particular Ube2W, are still able to bind the E2 binding site of the RING domain. However, the bound Ube2W is inhibited from conjugating ubiquitin to the N-terminus of the fusion protein.

The E2 binding site of the RING domain retains its ability to bind E2 enzymes, preferably retains the ability to bind Ube2W.

In one embodiment the first RING domain is at the N-terminal end of the fusion protein and the adaptor domain is located at the C-terminal end of the RING domain. Alternative embodiments may comprise the adaptor domain at the N-terminal of the fusion protein.

In one embodiment the fusion protein is N-terminally acetylated, i.e. the fusion protein comprises an acetyl group at its N-terminal residue. Capping the N-terminus of the fusion protein with an acetyl group, prevents the N-terminus from being ubiquitinated and the fusion protein from being degraded, for example during Trim-Away.

In some embodiments the N-terminus of the fusion protein is capped with other chemical moieties which prevent the N-terminus being ubiquitinated. The chemical moiety is covalently coupled to the N-terminus of the fusion protein. The chemical moiety inhibits Ube2W ubiquitination of the fusion protein. Chemical moieties that may be conjugated to the N-terminal also include, in addition to acetyl, other amine reactive moieties, for example methyl. Therefore, other modifications include when the fusion protein is N-terminally methylated, i.e. the fusion protein comprises a methyl group at its N-terminal residue.

In some embodiments the N-terminal of the fusion protein comprises an N-Acetyltransferase recognition site. An N-Acetyltransferase recognition site is a short amino acid sequence which N-Acetyltransferase enzymes will recognise. Preferably the recognition site comprises the sequence DDDI (SEQ ID NO: 14), or EEEI (SEQ ID NO: 15), more preferably DDDI. The presence of these sites allows acetylation of the fusion protein, such that the fusion protein comprises an acetyl group at its N-terminus.

In some embodiments the N-terminal of the fusion protein can undergo N-terminal cyclisation, preferably the fusion protein can undergo N-pyroglutamate cyclisation. In order to facilitate N-terminal cyclisation of the fusion protein, the fusion protein can comprise a glutamic acid or glutamine as the N-terminal residue of the fusion protein. In some embodiments the N-terminal glutamine is part of the sequence GFA at the N-terminus of the fusion protein.

Once the fusion protein has undergone N-terminal cyclisation the resultant fusion protein will comprise an N-terminal pyroglutamine as the N-terminal residue. Therefore, in some embodiments the fusion protein comprises an N-terminal pyroglutamate residue. Pyroglutamate as used herein refers broadly to the amino acid derivative in which the free amino group of glutamic acid or glutamine cyclizes to form a lactam. Without being bound by theory it is thought that presence of a pyroglutamate at the N-terminus of the fusion protein protects the fusion protein from N-terminal autoubiquitination via the E2 enzyme Ube2W.

In some embodiments the N-terminal amino acids of the fusion protein are substituted or modified with an amino acid or amino acid sequence that inhibits the ability of E2 enzymes, for example Ube2W, to ubiquitinate the fusion protein. In some embodiments the N-terminal amino acid may be substituted with an amino acid sequence that inhibits Ube2w ubiquitination, preferably E2 enzyme ubiquitination of the fusion protein. In some embodiments, amino acids at positions 1, 2, 3, 4 and 5 are modified or substituted to provide a sequence that inhibits Ube2W ubiquitination of the fusion protein. Preferably at least the amino acids at positions 1, 2, and 3, more preferably at least the amino acid at position 1 is substituted or modified. For example, in one embodiment the N-terminal amino acids, may be substituted with, a polyproline sequence, i.e. amino acids at positions 1, 2, 3 and 4 and 5 may be substituted with a polyproline sequence, or other sequence capable of blocking Ube2W ubiquitination, preferably E2 enzyme ubiquitination, of the fusion protein. The stretch of amino acids may replace an equivalent number of amino acids at the start of the fusion protein. In some embodiments the N-terminus of the fusion protein is modified by adding a sequence to the N-terminal that blocks Ube2W ubiquitination, preferably E2 enzyme ubiquitination, of the fusion protein, e.g. added N-terminally to the first RING domain or the adaptor domain. Therefore, in some embodiments the inventions provide fusion proteins comprising a RING domain and adaptor domain, that are incapable of being ubiquitinated.

Fusion proteins of some embodiments of the invention can be represented as:

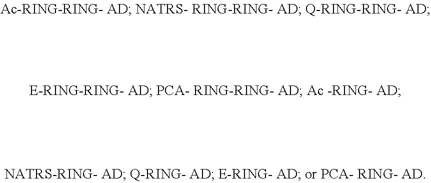

Wherein RING=Ring Domain, AD=Adaptor domain, Ac=acetyl group; NATRS=N-Acetyltransferase recognition site, E=glutamic acid, Q=glutamine, PCA=pyroglutamate, and wherein linker sequence may optionally be present between domains. Although the fusion proteins are represented with the Adaptor domain located N-terminally to the RING domain(s), the Adaptor Domain and RING domain(s) can be in any order as long as the N-terminus of the fusion protein comprises the modification to inhibit N-terminal autoubiquitination, e.g. so that Ube2W is unable ubiquitinate the fusion protein.

The RING domains of the fusion protein may be derived from any suitable polypeptide. RING domains are known in the art and were described in Freemont P S et al (1991) and function as E3 ligases (Meroni G and Roux G, 2005).

The RING domains used in the fusion proteins of the invention have E3 ubiquitin ligase activity. The RING domain of TRIM21 is an E3 ubiquitin ligase and targets ubiquitin conjugating enzymes to the substrate. Members of the RING (Really Interesting New Gene) domain family typically have the consensus sequence Cys-X₂-Cys-X(₉₋₃₉)-Cys-X(₁₋₃)-His-X(₂₋₃)-(Ans/Cys/His)-X₂-Cys-X(₄₋₄₈)-Cys-X₂-Cys (Deshaies R J and Joazeiro C, 2009). RING E3 ligase domains are found in a variety of proteins. Other RING domains include a RING domain from a protein X-linked mammalian inhibitor of apoptosis (XIAP) and a RING domain of DER3/Hrd1. Therefore, the use of RING domains derived from other protein families in the fusion proteins are also encompassed. The invention is particular applicable to RING domains that may be capable of self-ubiquitination, i.e. have self-ubiquitination activity.

Preferably the RING domains of the fusion protein are derived from a TRIM polypeptide. The TRIM family comprises a large number of RING E3 ligases (Marin, I. et al, 2012). In a preferred embodiment the RING domain is derived from a TRIM21 polypeptide, preferably human TRIM21. The sequence of human TRIM21 is set forth in SEQ ID NO: 1 (Uniprot: P19474).

(SEQ ID NO: 1)
MASAARLTMMWEEVTCPICLDPFVEPVSIECGHSFCQECISQVGK
GGGSVCPVCRØRFLLKNLRPNRQLANMVNNLKEISQEAREGTQGE
RCAVHGERLHLFCEKDGKALCWVCAQSRKHRDHAMVPLEEAAQEY
QEKLQVALGELRRKQELAEKLEVEIAIKRADWKKTVETQKSRIHA
EFVQQKNFLVEEEQRQLQELEKDEREQLRILGEKEAKLAQQSQAL
QELISELDRRCHSSALELLQEVIIVLERSESWNLKDLDITSPELR
SVCHVPGLKKMLRTCAVHITLDPDTANPWLILSEDRRQVRLGDTQ
QSIPGNEERFDSYPMVLGAQHFHSGKHYWEVDVTGKEAWDLGVCR
DSVRRKGHFLLSSKSGFWTIWLWNKQKYEAGTYPQTPLHLQVPPC
QVGIFLDYEAGMVSFYNITDHGSLIYSFSECAFTGPLRPFFSPGE
NDGGKNTAPLTLCPLNIGSQGSTDY

The RING domain of human TRIM21 comprises at least amino acids 3-81 of human TRIM21 sequence as set forth in SEQ ID NO: 1, preferably amino acids 1 to 85 of human TRIM21 amino acid sequence as set forth in SEQ ID NO: 1. The RING domain comprising amino acid 1 to 85 of human TRIM21 comprises the sequence:

(SEQ ID NO: 2)
MASAARLTMMWEEVTCPICLDPFVEPVSIECGHSFCQECISQVGK
GGGSVCPVCRQRFLLKNLRPNRQLANMVNNLKEISQEARE

Therefore, in one embodiment of the invention the RING domain comprises amino acids 3-81 of SEQ ID NO: 2, preferably amino acid residues 1-81 of SEQ ID NO: 2 or variant thereof. In one embodiment the RING domain comprises the sequence of SEQ ID NO: 2 or a variant thereof, preferably the RING domain of the fusion protein consists of the sequence of SEQ ID NO: 2 or a variant thereof.

Amino acids 3-81 of human TRIM21 comprises the sequence:

(SEQ ID NO: 3)
SAARLTMMWEEVTCPICLDPFVEPVSIECGHSFCQECISQVGKGG
GSVCPVCRØRFLLKNLRPNRQLANMVNNLKEISQ

Amino acids 1-81 of human TRIM21 comprises the sequence:

(SEQ ID NO: 4)
MASAARLTMMWEEVTCPICLDPFVEPVSIECGHSFCQECISQVGK
GGGSVCPVCRQRFLLKNLRPNRQLANMVNNLKEISQ

In some embodiment the RING domain comprises amino acid residues 2-81 of human TRIM21. In some embodiments the RING domain consists of amino acid residues 2-81 of human TRIM21.

Amino acids 2-81 of human TRIM21 comprises the sequence:

(SEQ ID NO: 5)
ASAARLTMMWEEVTCPICLDPFVEPVSIECGHSFCQECISQVGKG
GGSVCPVCRØRFLLKNLRPNRQLANMVNNLKEISQ

Preferably the variant sequence has at least 60% identity to the reference sequence, using the default parameters of the BLAST computer program (Atschul et al., 1990. provided by HGMP (Human Genome Mapping Project)), at the amino acid level. More preferably, the variant sequence of SEQ ID NO: 2 may have at least 65%, 70%, 75%, 80%, 85%, 90% and still more preferably 95% (still more preferably at least 99%) identity, at the amino acid level, to the sequence of SEQ ID NO:2.

“Identity” as known in the art is the relationship between two or more polypeptide sequences or two or more polynucleotide sequences, as determined by comparing the sequences. In the art, identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., 1984, BLASTP, BLASTN, and FASTA (Atschul et al., 1990)).

The N-terminal methionine of the expressed fusion protein may be co- or post-translationally cleaved from the expressed protein, e.g. by a Methionine amino peptidase. Therefore, wherein it is referred to the N-terminal amino acid of the fusion protein being modified, it also includes a fusion protein wherein it is the N-terminal residue of the fusion protein after excise of the methionine residue and other co- or post-translation processing of the expressed fusion protein that is modified, e.g. residue 2 of the SEQ ID NO: 2. Therefore, wherein the RING domain is at the N-terminal of the fusion protein in some embodiments it will be Arginine residue of RING domain that will modified, e.g. it will be acetylated or modified by other means to inhibit the ability of Ube2W to ubiquitination the fusion protein.

In some embodiments RING domains from a TRIM polypeptide other than TRIM21 can be used, for example a RING domain from TRIM5, TRIM7, TRIM 19, TRIM25, TRIM28 and/or TRIM32, preferably a RING domain from TRIM5 may be used.

The fusion protein comprises at least one RING domain, i.e. 1, 2, 3 or more RING domains, preferably the fusion comprises 2 or 3 RING domains, more preferably 2 RING domains.

In some embodiments the fusion protein comprises a second RING domain, wherein the second RING domain is between the first RING domain and the adaptor sequence. By providing a fusion protein comprising at least two RING domains and an adaptor domain the fusion protein is capable of forming part of a catalytic RING topology that enables protein degradation of a target protein.

Fusion proteins comprising two RING domains may be more efficient degraders of target protein than corresponding fusion proteins comprising only one RING domain, however dual RING fusion proteins are constitutively active even in the absence of substrate (e.g. under steady state conditions). This means that such fusion proteins are faster and more efficient, however they have a shorter half-life. Therefore, inhibiting N-terminal autoubiquitination, e.g. by blocking the N-terminal of fusion proteins comprising a RING-RING format is particularly beneficial for a RING-RING fusion protein format. Such RING-RING fusion proteins which are incapable of N-terminal autoubiquitination have both longer cellular half-life and improved function, as compared to a single RING domain fusion protein that is able to constitutively undergo N-terminal ubiquitination, and a longer half-life in comparison to a corresponding RING-RING fusion protein that does have autoubiquitination activity.

When the fusion comprises two RING domains, the adaptor domain is preferably at the C-terminal end of the first and second RING domains. The separate domains of the fusion protein can be provided in the order N-terminus to C-terminus of RING Domain-RING Domain-Adaptor Domain. In such an embodiment the amino acid sequence of the first RING domain is linked to the N-terminal of the second RING domain and the adaptor domain is linked to the C-terminal domain of the second RING domain. Such fusion proteins comprising at least two RING domains are described in WO2022/175549, wherein the adaptor protein is described as a protein targeting domain, the contents of which is incorporated herein by reference.

When the fusion protein comprises two RING domains, the RING domains have sequences capable of dimerizing with each other to form a RING dimer. Preferably the RING domains comprise the same sequence. In one embodiment the first RING domain and second RING domain both comprise the sequence of SEQ ID NO: 2. If the RING domains comprise different sequences, at least the sequences of the first and second RING domains should be capable of dimerizing with each other to form a RING dimer. In one embodiment the first RING domain comprises the sequence of SEQ ID NO: 2 and the second RING domain comprises a variant sequence of SEQ ID NO: 2, or vice versa. The variant sequence may have at least 65%, 70%, 75%, 80%, 85%, 90% and preferably 95% (still more preferably at least 99%) identity, to the sequence of SEQ ID NO:2.

The fusion protein comprises an adaptor domain. The adaptor domain helps localise the fusion protein in proximity with the target substrate (i.e. a target protein to be degraded). The adaptor domain is a polypeptide sequence capable of locating the fusion protein with the target substrate. The adaptor domain may associate with the target protein directly or may bind an antibody or antibody thereof that binds the target protein. The adaptor protein may be located at the C-terminal or N-terminal end of the construct, preferably the adaptor protein is located at the C-terminal end of the fusion protein.

In one embodiment the adaptor domain is referred to as a protein targeting domain. The protein targeting domain directs the fusion protein to the target protein (substrate) to be degraded, also referred to as a protein of interest. The protein targeting domain, binds the target protein or antibody or fragment thereof or antibody mimetic binding the same, and may also be referred to as a “protein binding domain”. The protein targeting domain may either bind the target protein directly to form a Fusion protein-Target protein complex, or bind to an antibody, antibody fragment thereof or antibody mimetic binding the target protein to form a Fusion protein-Antibody-Target protein complex. The protein targeting domain is preferably connected to the C-terminal end of the RING domain.

In one embodiment the protein targeting domain is the PRYSPRY domain. In one such an embodiment the fusion protein comprises a first RING domain and a PRYSPRY domain. In another embodiment the fusion protein comprises a first RING domain; a second RING domain; and a PRYSPRY domain. In another embodiment the fusion protein comprises a first RING domain; a second RING domain; and a PRYSPRY domain. The PRYSPRY domain is located at the C-terminal or N-terminal end of the RING domains (e.g. RING-PRYSPRY, RING-RING-PRYSPRY, RYSPRY-RING-RING or PRYSPRY-RING).

The N-terminal of the fusion protein is modified to prevent autoubiquitination of the fusion protein. For example, as discussed above the N-terminal of the first RING is N-acetylated (Ac), the fusion protein comprises a N-Acetyltransferase recognition site (NATRS), or the fusion protein comprises a N-terminal glutamic acid (E), glutamine (Q) or pyroglutamate (PCA) residue, such constructs can be represented as:

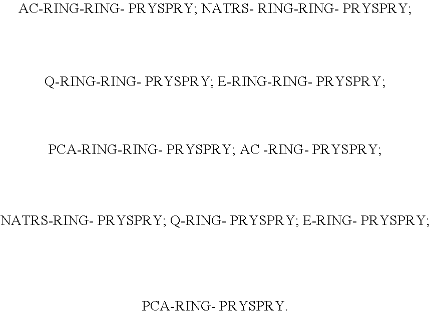

Although the fusion proteins are represented with the Adaptor domain located N-terminally to the RING domain(s), the Adaptor Domain and RING domain(s) can be in any order as long as the N-terminal of the fusion protein comprises the modification to inhibit N-terminal autoubiquitination, e.g. so that Ube2W is unable to ubiquitinate the fusion protein.

The PRYSPRY domain can be derived from a TRIM polypeptide preferably TRIM21, more preferably human TRIM21. The PRYSPRY domain is comprised of the PRY and SPRY regions at positions 286-337 and 339-465 of the human TRIM21 amino acid sequence as set forth in SEQ ID NO: 1.

Amino acids 286-337 of human TRIM21 are:

(SEQ ID NO: 7)
AVHITLDPDTANPWLILSEDRRQVRLGDTQQSIPGNEERFDSYPM
VLGAQHF

Amino acids 339-465 of human TRIM21 are:

(SEQ ID NO: 8)
SGKHYWEVDVTGKEAWDLGVCRDSVRRKGHFLLSSKSGFWTIWLW
NKQKYEAGTYPQTPLHLQVPPCQVGIFLDYEAGMVSFYNITDHGS
LIYSFSECAFTGPLRPFFSPGENDGGKNTAPLTLCPL

Preferably the PRYSPRY domain comprises the sequence:

(SEQ ID NO: 9)
AVHITLDPDTANPWLILSEDRRQVRLGDTQQSIPGNEERFDSYPM
VLGAQHFHSGKHYWEVDVTGKEAWDLGVCRDSVRRKGHFLLSSKS
GFWTIWLWNKQKYEAGTYPQTPLHLQVPPCQVGIFLDYEAGMVSF
YNITDHGSLIYSFSECAFTGPLRPFFSPGENDGGKNTAPLTLCPL

In one embodiment of the invention, the PRYSPRY domain comprises the sequence of SEQ ID NO: 9 or a variant thereof. Preferably the variant sequence has at least 60% identity to the reference sequence, using the default parameters of the BLAST computer program (Atschul et al., J. Mol. Biol. 215, 403-410 (1990) provided by HGMP (Human Genome Mapping Project), at the amino acid level. More preferably, the variant sequence of SEQ ID NO: 9 may have at least 65%, 70%, 75%, 80%, 85%, 90% and preferably 95% (still more preferably at least 99%) identity, at the amino acid level, to the sequence of SEQ ID NO:9.

The PRYSPRY domain of the fusion proteins binds to the Fc of an antibody or antibody fragment thereof, for example the Fc region of a human IgG1. The fusion protein binds the antibody bound to the target protein.

The Fc is a dimer and therefore can be bound by two PRYSPRY domains. The PRYSPRY domain of a first fusion protein binds one of the monomers of the Fc, whilst the PRYSPRY domain of a second fusion protein binds the second monomer of the Fc. This co-localises two fusion proteins bringing the RING dimers of each fusion protein into close proximity, so that one RING dimer of one fusion protein is available to mediate the ubiquitination of the other RING dimer.

In a further embodiment of the invention the protein targeting domain is an antibody, antibody fragment thereof, or antibody mimetic. Preferably the antibody fragment molecule is selected from the group consisting of a Fab, Fab′, F(ab′)2, scFab, Fv, scFV, dAB, VL fragments thereof, VH fragments thereof and sdAb (i.e. nanobodies) such as VHH fragments thereof. Preferably an scFV or VHH.

In one embodiment the fusion protein comprises a RING domain; and a VHH domain, wherein the RING domain is derived from a TRIM polypeptide, preferably TRIM21, wherein the VHH binds to a protein of interest. Preferably the VHH is at the C-terminal end of the RING domain. Preferably the fusion protein does not comprise a coiled-coil domain and/or a B-box domain derived from TRIM located between the VVH domain and the RING domain, more preferably the fusion protein does not comprise any coiled-coil domain or B-box domain sequence. The N-terminus of the fusion protein is modified to prevent autoubiquitination of the fusion protein. For example, as discussed above the N-terminus of the first RING is N-acetylated (Ac), the fusion protein comprises a N-Acetyltransferase recognition site (NATRS), or the fusion protein comprises a N-terminal glutamic acid (E), glutamine (Q) or pyroglutamate (PCA) residue, such construct can be represented as:

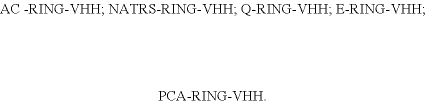

Although the fusion proteins are represented with the VHH located N-terminally to the RING domain, the VHH and RING domain can be in any order as long as the N-terminal of the fusion protein comprises the modification to inhibit N-terminal autoubiquitination, e.g. so that Ube2W is unable to bind the fusion protein. When the fusion protein is in the order VHH-RING, it will be the N-terminal of the VHH that may N-acetylated.

In one preferred embodiment the fusion protein comprises a first RING domain; a second RING domain; and a VHH domain, wherein the RING domains are derived from a TRIM polypeptide, preferably TRIM21, wherein the VHH binds to a protein of interest. Preferably the VHH is at the C-terminal end of the first and second RING domains. Preferably the fusion protein does not comprise a coiled-coil domain and/or a B-box domain derived from TRIM located between the VVH domain and the second RING domain, more preferably the fusion protein does not comprise any coiled-coil domain or B-box domain sequence. The N-terminal of the fusion protein is modified to prevent autoubiquitination of the fusion protein. For example, as discussed above the N-terminal of the first RING is N-acetylated (Ac), the fusion protein comprises a N-Acetyltransferase recognition site (NATRS), or the fusion protein comprises a N-terminal glutamic acid (E), glutamine (Q) or pyroglutamate (PCA) residue, such construct can be represented as:

AC-RING-RING-VHH; NATRS-RING-RING-VHH; Q-RING-RING-VHH; E-RING-RING-VHH; PCA-RING-RING-VHH.

Although the fusion proteins are represented with the VHH located N-terminally to the RING domains, the VHH and RING domains can be in any order as long as the N-terminal of the fusion protein comprises the modification to inhibit N-terminal autoubiquitination, e.g. so that Ube2W is unable to bind the fusion protein. When the fusion protein is in the order VHH-RING-RING, it will be the N-terminal of the VHH that may N-acetylated.

The antibody, antibody fragment thereof or antibody mimetic, for example the VHH, of the fusion protein specifically binds to the target protein. The fusion protein directly binds the target protein to be degraded at a target sequence of the target protein. Many proteins are oligomeric (or at least dimers) or part of a protein complex, therefore the antibody domain of a first fusion protein can bind one of the monomers of the oligomer or protein complex, whilst the antibody domain of a second fusion protein binds a second monomer of the oligomer or protein complex.

In one embodiment the target protein can be a protein having a pathogenic form and a non-pathogenic form. The protein targeting domain binds the pathogenic form but does not bind the non-pathogenic form of the protein. The pathogenic form of the target protein may comprise a repeat domain or is a multimeric form of the protein.

The target protein may be an intracellular protein selected from the group comprising of huntingtin and tau. If the intracellular protein is huntingtin, in one embodiment the protein target domain of the fusion protein binds to a poly-glutamate sequence of huntingtin.

In one embodiment the adaptor domain encodes for a protein or fragment thereof that is capable of locating the RING domain to the substrate.

Preferably the fusion protein does not comprise a B-box domain and a coiled-coil domain of TRIM21 located between first RING domain and the adaptor domain. In one embodiment wherein the fusion comprises two RING domains, the fusion protein may not comprise a B-box domain and a coiled-coil domain derived from any protein located between the second RING domain the adaptor domain, or between the first and second RING domains. In one embodiment the fusion protein does not comprise a B-box domain, such as a B-box domain derived from TRIM21, and preferably does not comprise a B-box domain derived from any protein. In one embodiment the fusion does not comprise a coiled-coil domain derived from TRIM21, and preferably does not comprise a coiled-coil domain derived from any protein.

The B-box domain of human TRIM21 comprises amino acid 91 to 128 of the human TRIM21 amino acid sequence as set forth in SEQ ID NO: 1. The coiled-coil domain of human TRIM21 comprises amino acids 128 to 238 of the human TRIM21 amino acid sequence as set forth in SEQ ID NO: 1.

The B-box domain can comprise the sequence:

(SEQ ID NO: 10)
RCAVHGERLHLFCEKDGKALCWVCAQSRKHRDHAMVPL

Therefore, in one embodiment the fusion protein does not comprise the sequence of SEQ ID NO: 10 or a variant thereof.

The coiled coil domain can comprise the sequence:

(SEQ ID NO: 11)
EEAAQEYQEKLQVALGELRRKQELAEKLEVEIAIKRADWKKTVET
QKSRIHAEFVQQKNFLVEEEQRQLQELEKDEREQLRILGEKEAKL
AQQSQALQELISELDRRCHS

Therefore, in one embodiment the fusion protein does not comprise the sequence of SEQ ID NO: 11 or a variant thereof.

Preferably the fusion protein does not comprise the sequence of SEQ ID NO: 10 and SEQ ID NO: 11 or functional variants thereof. Preferably the variant sequence has at least 60% identity to the reference sequence, using the default parameters of the BLAST computer program (Atschul et al., 1990), at the amino acid level. More preferably, the variant sequence of SEQ ID NO: 10 or 11 may have at least 65%, 70%, 75%, 80%, 85%, 90% and preferably 95% (still more preferably at least 99%) identity, at the amino acid level, to the sequence of SEQ ID NO:10 or 11.

In an embodiment having two RING domains, by not including the coiled-coil domain and the B-box domain between the second RING domain and the adaptor domain, assists in allowing the RING dimers of a fusion protein to be in close proximity with the RING dimers of a second fusion protein co-localised on the target protein (or antibody binding the target protein).

However, in some embodiments the fusion construct may comprise a coiled-coil domain, a B-box domain, or a coiled-coil domain and a B-box domain. If a coiled-coil domain and/or a B-box are present in the fusion protein comprising two RING domains, they should be located at a sufficient distance from the adaptor domain and RING domains such that the RING dimer of a first fusion protein can still be in close proximity to the RING dimer of a second fusion protein, co-localised on the target protein (or antibody binding the target protein), for example when both are bound to the same Fc.

Linker sequences may be provided between the RING domains and adaptor domain and between each RING domain present in the fusion protein.

The linker sequences may be derived from a sequence of a TRIM polypeptide, wherein the linker sequence does not encode for the coiled-coil domain and/or the B-box domain of a TRIM polypeptide In other embodiments standard linker sequences known in the art may be also be used, for example polyglycine or polyserine amino acid sequences may be used, or linker sequences comprising the combination of glycine and serine residues, e.g. a linker having the sequence GSGGGGS (SEQ ID NO: 12). The linker length can vary in size.

However, in an embodiment comprising two RING domains, the linker sequence between the two RING domains should be of sufficient length to provide flexibility to the fusion protein and enable dimerization of the two RING domains present. In one embodiment the linker sequence between the RING domains is between 1-50 amino acid in length, preferably 1-35, 1-30, 1-25, 1-20, 1-15 or 1-10 amino acids in length. More preferably the linker is 1-6 amino acids in length, for example 1, 2, 3, 4, 5, or 6 amino acids in length. In some embodiments no linker may be present between the first and second RING domains.

In an embodiment wherein the fusion comprises two RING domains, the linker sequence between the RING domain and the adaptor domain, should of be a length sufficient that enables the RING dimer of first fusion protein to be in close proximity to the RING dimer of a second fusion protein when co-localised on the target protein (or antibody binding the target protein). The linker should be of sufficient length to enable formation of the catalytic RING topology with a RING domain of a second protein.

In one embodiment the linker sequence between the adaptor domain and the RING domain is between 5 and 50 amino acids in length, preferably 5-40, 5-30, 5-25, 10-25, 15-25, 15-20 or 10-20 amino acids in length. More preferably the linker is between 10-20 amino acids in length.

In one embodiment the linker sequences may be derived from a sequence of a TRIM polypeptide, wherein the linker sequence does not encode for the coiled-coil domain and/or the B-box domain of a TRIM polypeptide. For example, the linker sequence provided between the RING domain and adaptor domain may comprise the sequence GTQGERGLKKMLRTC (SEQ ID NO: 13). In one embodiment the sequence consists of the sequence GTQGERGLKKMLRTC (SEQ ID NO: 13).

Therefore one embodiment of the invention comprises a fusion protein comprising a first RING domain; a second RING domain; an adaptor domain located at the C-terminal end of the first and second RING domains, and a linker sequence between the RING domains and the adaptor domain, wherein the RING domains are derived from a TRIM polypeptide, preferably TRIM21, and preferably wherein the fusion protein does not comprise a coiled-coil domain or a B-box domain, and wherein fusion protein is incapable of N-terminal autoubiquitination. Preferably the first RING domain is N-acetylated (Ac), the fusion protein comprises a N-Acetyltransferase recognition site (NATRS) at its N-terminus, or the fusion protein comprises a N-terminal glutamic acid (E), glutamine (Q) or pyroglutamate (PCA) residue.

Another embodiment of the invention comprises a fusion protein comprising one RING domain; an adaptor domain located at the C-terminal end of the RING domain, and a linker sequence between the RING domain and the adaptor domain, wherein the RING domain is derived from a TRIM polypeptide, preferably TRIM21 and preferably wherein the fusion protein does not comprise a coiled-coil domain or a B-box domain, and wherein fusion protein is incapable of N-terminal autoubiquitination. Preferably the N-terminal of the fusion protein is modified to prevent autoubiquitination of the fusion protein. Preferably the RING domain is N-acetylated (Ac), the fusion protein comprises a N-Acetyltransferase recognition site (NATRS) at its N-terminus, or the fusion protein comprises a N-terminal glutamic acid (E), glutamine (Q) or pyroglutamate (PCA) residue.

A “fusion protein” and a “fusion polypeptide” refer to a polypeptide having two or more portions covalently linked together, where each of the portions is a polypeptide having a specific property, which may be the same or different. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other.

The term “fusion protein” in this text means, in general terms, one or more proteins joined together by chemical means, including hydrogen bonds or salt bridges, or by peptide bonds through protein synthesis or both. Typically, fusion proteins will be prepared by DNA recombination techniques standard in the art and may be referred to herein as recombinant fusion proteins.

The invention also provides nucleic acid constructs encoding a fusion protein of the invention. The nucleic acid construct can comprise a first nucleic acid sequence encoding a first RING domain; and a second nucleic acid sequence encoding an adaptor domain. In some embodiments the nucleic acid construct can comprise a third nucleic acid sequence encoding a second RING domain, located between the first RING domain and adaptor domain. The nucleic acid can encode for a fusion protein that is incapable of autoubiquitination, and comprises an N-terminal which inhibits Ube2W ubiquitination of the fusion protein. Although the nucleic acid constructs of the encode fusion proteins that inhibit Ube2W ubiquitination of the fusion protein, the fusion proteins encoded by the nucleic construct are able to bind Ube2W.

In some embodiments the nucleic construct comprises a sequence that encodes for N-Acetyltransferase recognition site at the N-terminal of fusion protein. In some embodiments the nucleic acid construct encodes a fusion protein having the sequence DDDI (SEQ ID NO: 14) or EEEI (SEQ ID NO: 15) at its N-terminus.

In some embodiments the nucleic construct encodes for a fusion protein comprising a glutamine or glutamic acid residue as the N-terminal residue. In some embodiments the nucleic acid construct can further comprise a sequence that encodes a glutaminyl cyclase.

In some embodiments the nucleic acid construct encodes for a fusion protein having an amino acid sequence at its N-terminal that inhibits Ube2W ubiquitination of the fusion protein. The amino acid sequence may be in addition to the RING domain and Adaptor domain. Alternatively, the amino acid sequence may replace an N-terminal sequence of the RING or Adaptor domain (depending on which domain is located at the N-terminal). For example, when the nucleic acid construct encodes for a fusion protein sequence with the RING domain at the N-terminal, the nucleic construct encodes a RING domain, wherein the N-terminal resides of the RING domain have been substituted with an amino acid sequence that inhibits Ube2W ubiquitination of the fusion protein, for example the N-terminal residues of the RING domain may be substituted with a polyproline sequence. In another embodiment wherein the nucleic acid construct encodes for a fusion protein sequence with the RING domain at the N-terminal, the nucleic construct comprising an additional nucleic acid sequence that encodes an amino acid sequence that inhibits Ube2W ubiquitination of the fusion protein, wherein the amino acid sequence that inhibits Ube2W ubiquitination of the fusion protein is located at the N-terminal of the RING domain have been substituted with a sequence.

In some embodiments the nucleic acid construct does not encode for a coiled-coil domain; does not encode for or a B-Box domain or does not encode for a coiled-coil domain and a B-box domain.

There nucleic acid construct may be provided in the form of a vector, for example, an expression vector, and may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, lentiviruses, fowl pox viruses, pseudorabies viruses and retroviruses, and vectors derived from combinations thereof, such as those derived from plasmid and bacteriophage genetic elements, such as cosmids and phagemids.

Generally, any vector suitable to maintain, propagate or express nucleic acid to express a polypeptide in a host, may be used for expression in this regard. The vector may comprise a plurality of the nucleic acid constructs defined above, for example two or more. Preferably the vector is viral delivery vector, preferably an adenoassociated virus (AAV) vector or a lentivirus vector.

The nucleic acid construct of the invention preferably includes a promoter or other regulatory sequence which controls expression of the nucleic acid. The promoter or other regulatory sequences can be operably linked to the nucleic acid sequences encoding the domains of the fusion protein. Promoters and other regulatory sequences which control expression of a nucleic acid have been identified and are known in the art. The person skilled in the art will note that it may not be necessary to utilise the whole promoter or other regulatory sequence. Only the minimum essential regulatory element may be required and, in fact, such elements can be used to construct chimeric sequences or other promoters.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

The invention also provides hosts cell comprising such nucleic acid constructs. The fusion proteins may be expressed in a variety of cells.

The invention also provides a method for preparing fusion proteins of the invention, the method comprising cultivating or maintaining a host cell comprising the nucleic construct or vector described above under conditions such that said host cell produces the fusion protein, optionally further comprising isolating the fusion protein.

In one aspect of the invention there is provided a method producing the fusion protein of the invention.

The method comprising:
(a) culturing a host cell comprising a first vector encoding a fusion protein comprising a RING domain and an adaptor domain, under conditions to permit expression of the fusion protein; and
(b) obtaining the expressed protein from the host cell, wherein the fusion protein is incapable of N-terminal autoubiquitination.

In some embodiments the first vector also encodes for:
a N-Acetyltransferase recognition site at the N-terminal of the fusion protein, preferably wherein the N-Acetyltransferase recognition site is the sequence DDDI (SEQ ID NO:14) or EEEI (SEQ ID NO: 15);
glutamic acid residue or a glutamine reside at the N-terminal of the fusion protein; or the
the sequence QFA at the N-terminal of the fusion protein;
an amino acid sequence at the N-terminus of the fusion protein that inhibits the ubiquitination of the fusion protein by Ube2W, for example a polyproline sequence.

In some embodiments the first vector also encodes a glutaminyl cyclase and the fusion protein expressed comprises an N-terminal glutamic acid residue or an N-terminal glutamine residue, and the method comprises culturing the host cell under conditions to permit expression of the fusion protein and the glutaminyl cyclase.

For example, in one embodiment first vector also expresses a tev cleavage site with the fusion protein, and the second vector express a tev protease. The fusion protein can be expressed on a multicistronic vector that also expresses the glutaminyl cyclase. The first vector is co-transfected with the second vector. During expression the tev protease cleaves the RING fusion protein to expose the N-terminal glutamine, which is cyclised by the glutaminyl cyclase. The cyclised RING fusion protein having a pyroglutamate at their N-terminus is then purified, for example on the basis of a C-terminal His tag. In such an embodiment the following fusion proteins are expressed during the production of the fusion protein of the invention.
1. T7-RBS-ENLYVQQFA-R-R-PY-6His-Stop-RBS-bQC(E45Q)-Stop-T7T
2. T7PRBS-MBP-REVP(S219V)

Where T7P=T7 promoter, RBS=Ribosome binding site, ENLYVQ (SEQ ID NO: 16) is the tev site, QFA is the site for glutaminyl cyclase (QC), R=RING, PY=PRYSPRY domain, 6His=6 Histidines, Stop=stop codon, T7T=T7 terminator, MBP=maltose binding protein, TEVP=TEV protease.

In some embodiments the methods comprise the step of modifying the N-terminus of the expressed fusion protein. Modification of the N-terminus of the fusion protein can occur in a variety of ways.

In some embodiments the first vector also encodes for a N-Acetyltransferase recognition site at the N-terminus of the expressed fusion protein, and the method further comprises the step of incubating the fusion protein with an N-acetyl transferase (NAT) and acetyl-CoA. The NAT can add an acetyl group to the N-terminal of the fusion protein.

Capping the N-terminus of the fusion protein with an acetyl group, prevents the N-terminus from being ubiquitinated and the fusion protein from being degraded, for example during Trim-Away.

In some embodiments the first vector encodes a fusion protein having a glutamic acid residue or a glutamine reside at N-terminal of the expressed fusion protein, and the method further comprises the step of incubating the fusion protein with a glutaminyl cyclase. In some embodiments the vector encodes a fusion protein having the sequence QFA as its N-terminal sequence. The glutaminyl cyclase cyclises the free amino group of glutamic acid or glutamine to form a lactam, to provide a pyroglutamate as the N-terminal reside of the fusion protein. Capping the N-terminus of the fusion protein with pyroglutamate resides, prevents the N-terminal from being ubiquitinated and the fusion protein from being degraded, for example during Trim-Away.

In some embodiments the fusion protein may be expressed with N-terminal methionine. Such residue may be post-translationally cleaved from the expressed protein, e.g. by a Methionine amino peptidase. Therefore, wherein it is referred to the N-terminal amino acid of the fusion protein being modified, it also includes a fusion protein wherein it is the N-terminal residue of the fusion protein after excision of the methionine residue of the expressed fusion protein that is modified, e.g. residue 2 of the SEQ ID NO: 2. Therefore, wherein the RING domain is at the N-terminal of the fusion protein in some embodiments it will be the Arginine residue of RING domain that will modified, e.g. it will be acetylated or modified by other means to inhibit to prevent autoubiquitination via Ube2W, or may be substituted with an amino acid that inhibits the ability of Ube2W to ubiquitinate the fusion protein.

In some embodiments the method further comprises:
methylating the N-terminal amino acid of the expressed fusion protein; or
coupling a chemical moiety to the N-terminal amino acid of the expressed fusion protein, wherein the chemical moiety reduces the ability of E2 enzymes Ube2W to ubiquitinate the fusion protein.

Therefore, in some embodiments the methods provide fusion proteins that are incapable of being ubiquitinated.

Also provided is a pharmaceutical composition comprising the fusion protein or nucleic acid constructs of the invention. The pharmaceutical composition may contain a variety of pharmaceutically acceptable carriers and/or excipients. Suitable pharmaceutically acceptable carriers and/or excipients are known in the art. Pharmaceutical compositions of the invention may be for administration by any suitable method known in the art, including but not limited to intravenous, intramuscular, oral, or intraperitoneal. In preferred embodiments, the pharmaceutical composition may be prepared in the form of a liquid, gel, powder, tablet, capsule, or foam.

The fusion proteins and nucleic acid constructs of the invention may be used for therapy as a medicament. In one embodiment the invention also provides for the treatment of neurological disorders, for example Alzheimer's Disease or Huntington's Disease. In other embodiments the invention provides for the treatment of an infection, for example a viral infection such as HIV. In further embodiments the invention provides for the treatment of a trinucleotide repeat disorder, in particular trinucleotide repeat disorders wherein the trinucleotide repeat resides in the coding sequence of the gene. Trinucleotide repeat disorders that may be treated with the fusion proteins or nucleic acid constructs of the invention include Huntington disease, Dentatorubropallidoluysian atrophy and spinocerebellar ataxia.

The treatment of the neurological disorder, infection or trinucleotide repeat disorder comprises administering to the subject a fusion protein, nucleic acid or pharmaceutical composition of the invention.

In one embodiment the treatment involves administering a fusion protein comprising: a first RING domain; and an adaptor domain, wherein the fusion protein is incapable of N-terminal autoubiquitination. The adaptor domain is located at the C-terminal end of the first and second RING domains. Preferably the fusion protein administered does not comprise a coiled-coil domain or a B-box domain. In some embodiments the fusion protein comprises a second RING domain located between the first RING domain and the adaptor domain.

In one embodiment of the invention the treatment involves administering a nucleic acid construct comprising a first nucleic acid sequence encoding a first RING domain; and a second nucleic acid sequence encoding a protein targeting domain, wherein the nucleic acid encodes for a fusion protein incapable of N-terminal autoubiquitination. The nucleic acid construct encodes for a fusion protein wherein the protein targeting domain is located at the C-terminal end of the RING domain. Preferably the nucleic acid constructs administered do not comprise a sequence encoding for a B-box domain or a coiled-coil domain. In some embodiments the nucleic acid construct comprises a third nucleic acid sequence encoding a second RING domain located between the first RING domain and the adaptor domain.

When the disorder to be treated is a neurological disorder such as Alzheimer Disease, the adaptor protein may encode for a sequence that targets tau. In one embodiment the adaptor domain may encode for an antibody, antibody fragment thereof or antibody mimetic that specifically binds for tau. When the disorder to be treated is Huntington disease, the adaptor domain may encode for a sequence that targets huntingtin. In one embodiment the adaptor domain may encode for an antibody, antibody fragment thereof or antibody mimetic that specifically bind the polyglutamate sequence of huntingtin.

The nucleic acid construct according to the invention may also be administered by means of delivery vectors. These include viral delivery vectors, such as adenovirus, retrovirus or lentivirus delivery vectors known in the art. Other non-viral delivery vectors include lipid delivery vectors, including liposome delivery vectors known in the art.

Treatment includes both prophylaxis (prevention) and therapeutic treatment. The terms “treat”, “treating” or “treatment” (or equivalent terms) mean that the severity of the individual's condition is reduced or at least partially improved or ameliorated and/or that some alleviation, mitigation or decrease in at least one clinical symptom is achieved and/or there is an inhibition or delay in the progression of the condition and/or prevention or delay at the onset of a disease or illness.

The terms “patient”, “individual” or “subject” include human and other mammalian subjects that receive either prophylactic or therapeutic treatment with the fusion proteins or nucleic acid constructs described herein. Mammalian subjects include primates, e.g., non-human primates. Mammalian subjects also include laboratory animals commonly used in research, such as but not limited to, rabbits and rodents such as rats and mice.

The fusion proteins and nucleic acid constructs of the invention may also be used as a research tool, for example the degradation of proteins in a cell or sample.

Accordingly, in one embodiment of the invention there is provided a method of degrading a protein in a cell comprising administering a fusion protein or a nucleic acid of the invention. The cell may be an in vitro cell.

A further embodiment of the invention provides a method of degrading a protein in a sample comprising introducing a fusion protein or a nucleic construct of the invention into a sample.

In one embodiment the methods of degrading a protein in a cell or sample involve administering a fusion protein comprising: a first RING domain; and an adaptor domain, wherein the fusion protein is incapable of N-terminal autoubiquitination. The adaptor domain is preferably located at the C-terminus end of the RING Domain. Preferably the fusion protein administered does not comprise a coiled-coil domain or a B-box domain. In some embodiments the fusion protein comprises a second RING domain located between the first RING domain and the adaptor domain.

In one embodiment of the invention the methods of degrading a protein in a cell or sample involve administering a nucleic acid construct comprising a first nucleic acid sequence encoding a first RING domain; and a second nucleic acid sequence encoding an adaptor domain, wherein the nucleic acid encodes for a fusion protein incapable of N-terminal autoubiquitination. Preferably the nucleic acid construct administered does not comprise a sequence encoding for a B-box domain or a Coiled-coil domain. In some embodiments the nucleic acid construct comprises a third nucleic acid sequence encoding a second RING domain located between the first RING domain and the adaptor domain. The nucleic acid construct may have other features as described above.

An antibody, antibody fragment thereof, or antibody mimetic targeting a protein of interest, or a nucleic acid encoding the antibody, antibody fragment thereof, or antibody mimetic, may also be administered to the cell or sample. A “protein of interest” is a protein targeted for degradation. The antibody, antibody fragment thereof, or antibody mimetic may specifically bind the protein of interest.

The methods are particular useful for degrading proteins in cells that don't endogenously express TRIM21. The methods of are particularly useful in degrading intracellular proteins. However, in some embodiments an antibody will bind the protein of interest extracellularly, for example when targeting a pathogen, such as a virus. The antibody-target will be internalised in a cell, where the fusion protein will bind the antibody-target degrading the protein.

The fusion protein or nucleic acid can be introduced into the cell by transfection for example by injection, including microinjection or by electroporation, or transduction for example by the use of a viral delivery vector, for example an AAV vector. Other suitable delivery techniques for introducing the fusion protein and nucleic acid constructs into cells are known in the art.

The provided fusion proteins have increased cellular half-life relative to a corresponding fusion protein that is capable of autoubiquitination.

In one aspect of the invention there is provided a method of increasing cellular half-life of a fusion protein comprising a RING domain and an adaptor sequence where the adaptor sequence is capable of localising the RING domain with a substrate, the method comprising modifying the fusion protein such that it does not undergo N-terminal autoubiquitination.

By an “increased cellular half-life” it is meant that the cellular half-life of the modified fusion protein is increased relative to a corresponding fusion protein without the modification.

Modifying the fusion such that it is unable to undergo N-terminal autoubiquitination involves modifying the N-terminal of the fusion protein, as compared to an unmodified fusion protein capable of N-terminal autoubiquitination. Modification of the N-terminal results in a fusion protein that cannot be ubiquitinated by Ube2W. This is accomplished by rendering the reactive N-terminus of the fusion protein incapable of being covalently modified with ubiquitin by Ube2W. Modification of the N-terminal of the fusion protein can occur a variety of ways.

In one embodiment the method comprises incubating the fusion protein with an N-acetyl transferase (NAT) and acetyl-CoA to N-terminally acetylate the fusion protein. Capping the N-terminus of the fusion protein with an acetyl group, prevents the N-terminal from being ubiquitinated and the protein from being degraded, for example during Trim-Away.

The fusion protein can also be modified by introducing an N-Acetyltransferase recognition site to the N-terminal of the fusion protein. N-Acetyltransferase recognition sites include sequences such as DDDI and EEEI. In some embodiments the method can further comprise incubating the fusion protein with an N-acetyl transferase (NAT) and acetyl-CoA to N-terminally acetylate the fusion protein. The presence of the N-Acetyltransferase recognition site also makes the fusion protein a substrate for cytosolic N-acetyl transferase. The cytosolic N-Acetyltransferase can add an acetyl group to the N-terminus of the fusion protein.

Other techniques include methylating the N-terminal amino acid of the fusion protein or coupling chemical moiety to the N-terminal amino acid of the fusion protein, wherein the chemical moiety inhibits the ability of E2 enzymes, in particular Ube2W, to ubiquitinate the fusion protein.

The fusion protein can also be modified so that the fusion protein is capable of undergoing N-terminal cyclization. Preferable the method comprise modifying the fusion protein so that the fusion protein is capable of undergoing N-terminal pyroglutamate cyclisation.

In order to provide a fusion protein that can undergo N-terminal pyroglutamate cyclisation, the method can comprise introducing a glutamic acid or a glutamine residue to the N-terminal of the fusion protein. Preferably the method comprises encoding an N-terminal glutamic acid residue into the fusion protein.

Proteins beginning with glutamine or glutamic acid can undergo an N-terminal cyclization reaction to form a pyroglutamate at their N-terminus. Without being bound by theory it is thought that in a fusion protein having an N-terminal RING domain, the presence of a pyroglutamate at the N-terminus of the fusion protein protects the RING domain from N-terminal autoubiquitination via Ube2W.

This reaction can happen spontaneously or can be catalysed by glutaminyl cyclase (GS) enzymes. Pyroglutamate cyclisation of fusion proteins could be allowed to take place spontaneously by encoding an N-terminal glutamine or glutamate. In one embodiment the method may further comprise incubating the fusion protein with a glutaminyl cyclase. In some embodiments the method comprises introducing the sequence QFA to the N-terminal of the protein. Adding this QFA sequence onto a target protein and co-expressing with the GS enzyme allows the N-terminally cyclised proteins to be produced.

In one embodiment enzymatically catalysed cyclisation of the N-terminal glutamine of the fusion could be performed in vitro or co-translationally in cells as follows: A fusion protein comprising a RING domain is expressed with a tev cleavage site to leave an exposed glutamine at the N-terminus. It is expressed on a multicistronic vector that includes the glutaminyl cyclase. The plasmid is co-transfected with a second plasmid encoding the tev protease. During expression, the tev protease cleaves the RING fusion protein to expose the N-terminal glutamine, which is cyclised by the glutaminyl cyclase. The cyclised RING fusion protein having a pyroglutamate at their N-terminus is then purified, for example on the basis of a C-terminal His tag. In such an embodiment the following fusion proteins are expressed during the production of the fusion protein of the invention.

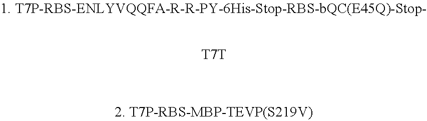

Where T7P=T7 promoter, RBS=Ribosome binding site, ENLYVQ (SEQ ID NO: 16) is the tev site, QFA is the site for glutaminyl cyclase (QC), R=RING, PY=PRYSPRY domain, 6His=6 Histidines, Stop=stop codon, T7T=T7 terminator, MBP=maltose binding protein, TEVP=TEV protease

A further method for modifying a fusion protein comprises substituting or modifying the N-terminal amino acids of the fusion protein, e.g. the N-terminal amino acid of the RING domain if located at the N-terminus, with an amino acid sequence that inhibits the ability of Ube2W to ubiquitinate the fusion protein. In some embodiments only the N-terminal amino acid may be substituted with an amino acid sequence that results in inhibition of Ube2w ubiquitination of the fusion protein.

In some embodiments amino acid at positions 1, 2, 3, 4 and 5 are modified or substituted to provide an amino acid sequence that inhibits the ubiquitination of the fusion protein by Ube2W. Preferably the method comprises modifying or substituting at least the amino acids at positions 1, 2, and 3, more preferably at least the amino acids at position 1. For example, in one embodiment the method comprises substituting the -N-terminal amino acids of the RING domain located at the N-terminus, with a polyproline sequence, e.g. the method can comprise substituting amino acids at positions 1, 2, 3 and 4 and 5 with a polyproline sequence, or other sequence capable of inhibiting Ube2W ubiquitination of the fusion protein. The stretch of amino acids may replace an equivalent number of amino acids at the start of the fusion protein, In some embodiments the method comprises adding an amino acid sequence that is capable of blocking Ube2W ubiquitination of the fusion protein to the N-terminal of the fusion protein e.g. wherein the fusion protein comprises the RING domain at the N-terminus, the method comprises adding the amino acid sequence to the N-terminal of the first RING domain.

The terms “comprising” or “comprises” may be substituted with the terms “consisting of”, “consists of”, “consisting essentially of” or “consists essentially of” and vice versa, wherever they occur herein.

The contents of all publications cited herein are incorporated herein by reference in their entirety into this application to more fully describe the state of the art to which this invention pertains.

The present invention will be further understood by reference to the following examples.

### Examples

### Methods

### Plasmids

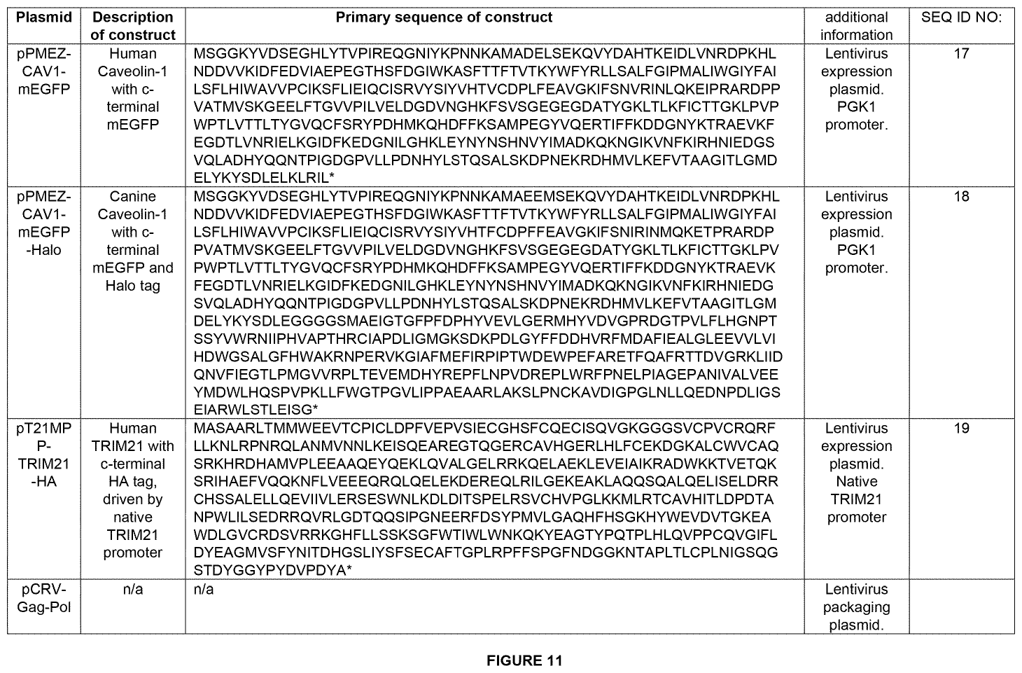

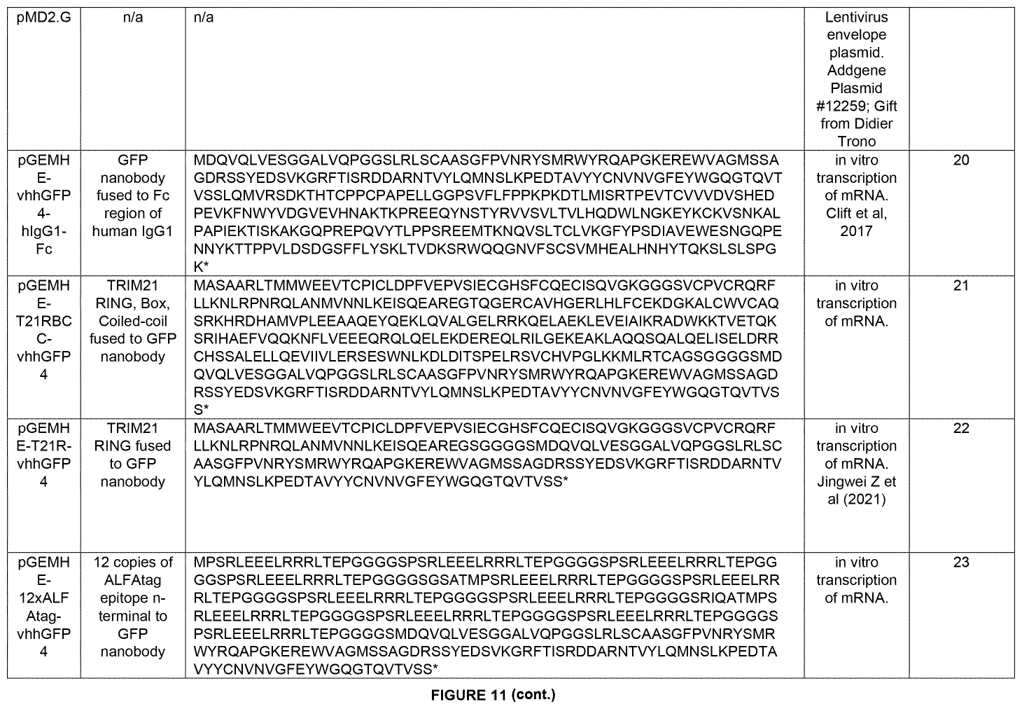

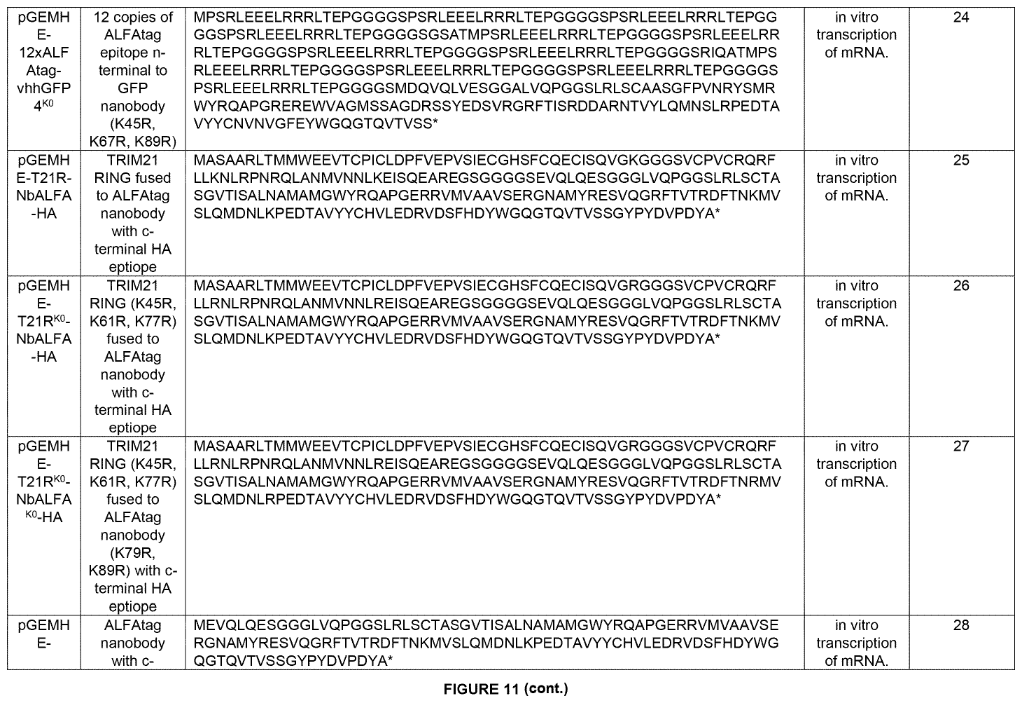

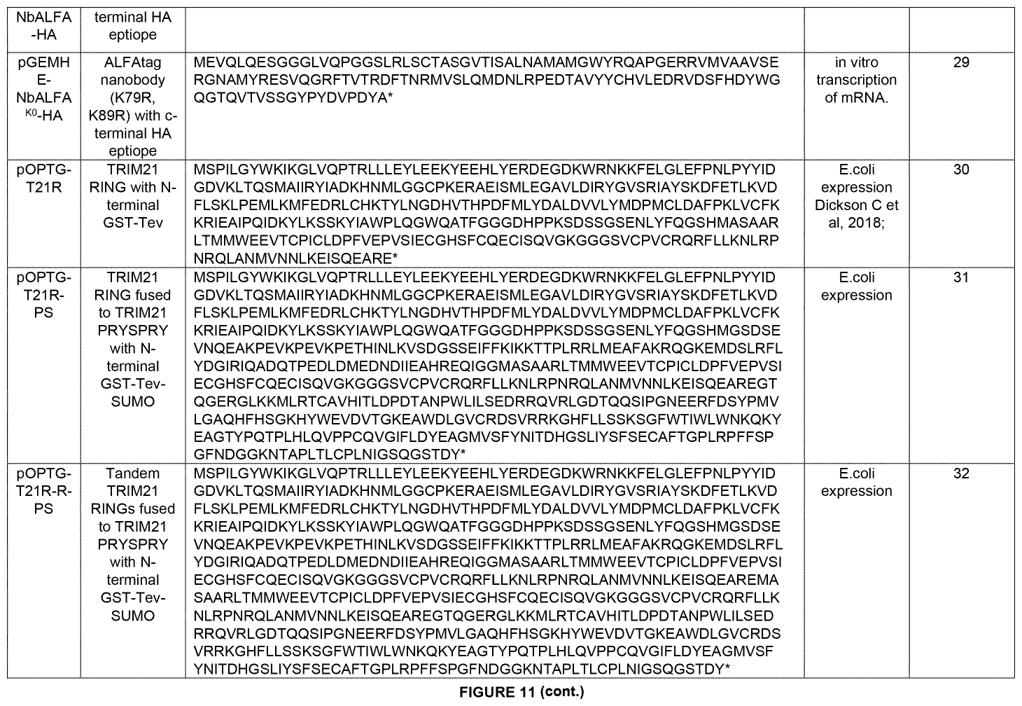

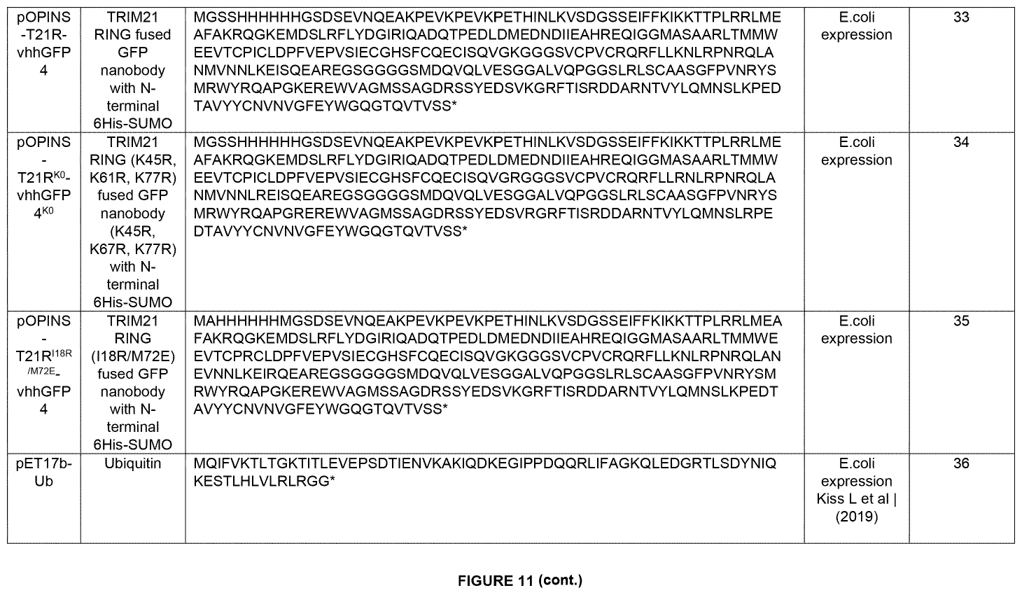

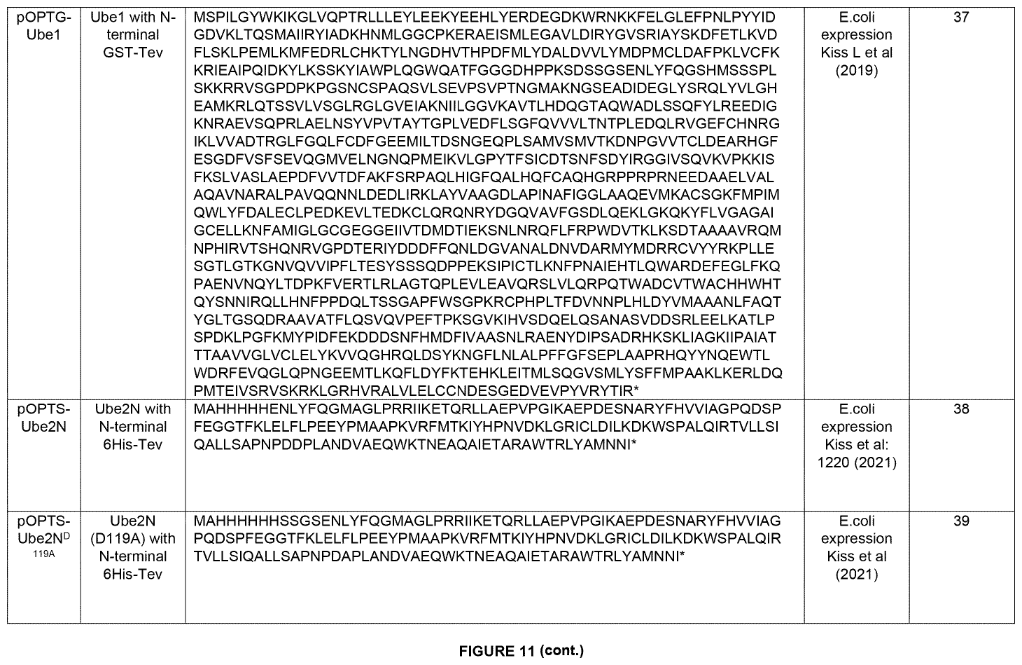

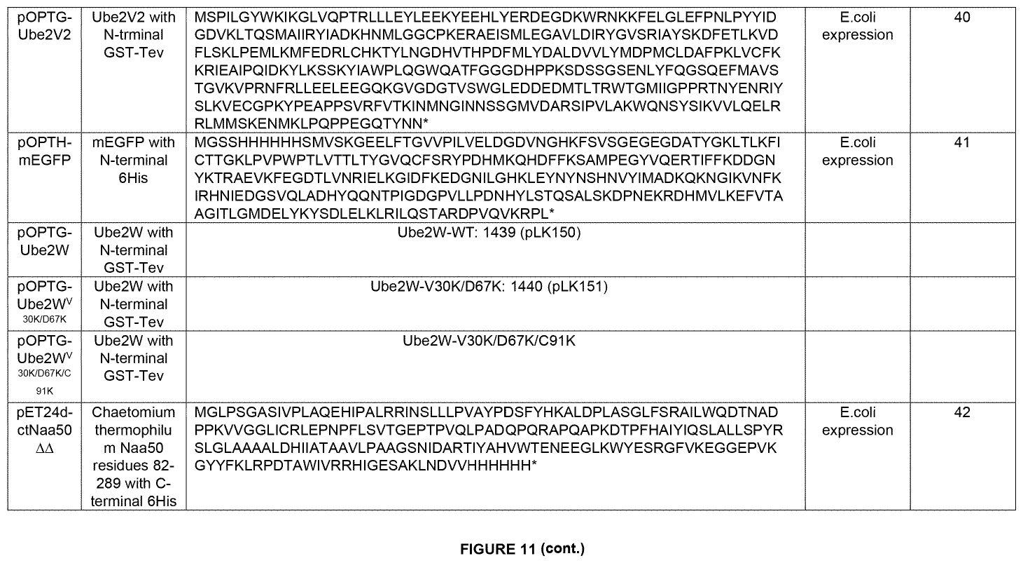

A full list of plasmids used in this study, including primary sequences of all constructs can be found in FIG. 11. For lentivirus production, constructs were inserted into a modified version of pSMPP (Addgene #104970) where the SFFV promotor and puromycin resistance sequences were replaced with PGK1 promoter and Zeocin resistance sequences respectively (pPMEZ). For in vitro mRNA transcription, constructs were inserted into pGEMHE (Liman et al., 1992), which contains UTR and polyA sequences for optimal mRNA stability and translation. For protein purification, constructs were inserted into derivations of the pOP and pET (Novagen) series of vectors.

### Lentivirus Production

Lentivirus particles were collected from HEK293T cell supernatant 3 days after co-transfection (FuGENE 6, Promega) of lentiviral plasmid constructs (FIG. 11) with HIV-1 GagPol expresser pcRV1 (a gift from Dr. Stuart Neil) and pMD2G, a gift from Didier Trono (Addgene plasmid #12259). Supernatant was filtered at 0.45 μm before storage at −80° C.

In Vitro Transcription of mRNA

pGEMHE plasmid constructs (FIG. 11) were linearised and 5′-capped mRNA was synthesised with T7 polymerase (NEB HiScribeT7 ARCA kit) according to manufacturer's instructions. mRNA concentration was quantified using a Qubit 4 fluorometer (ThermoFisher) and RNA Broad Range assay kit (ThermoFisher; Q10211).

### Protein Expression and Purification

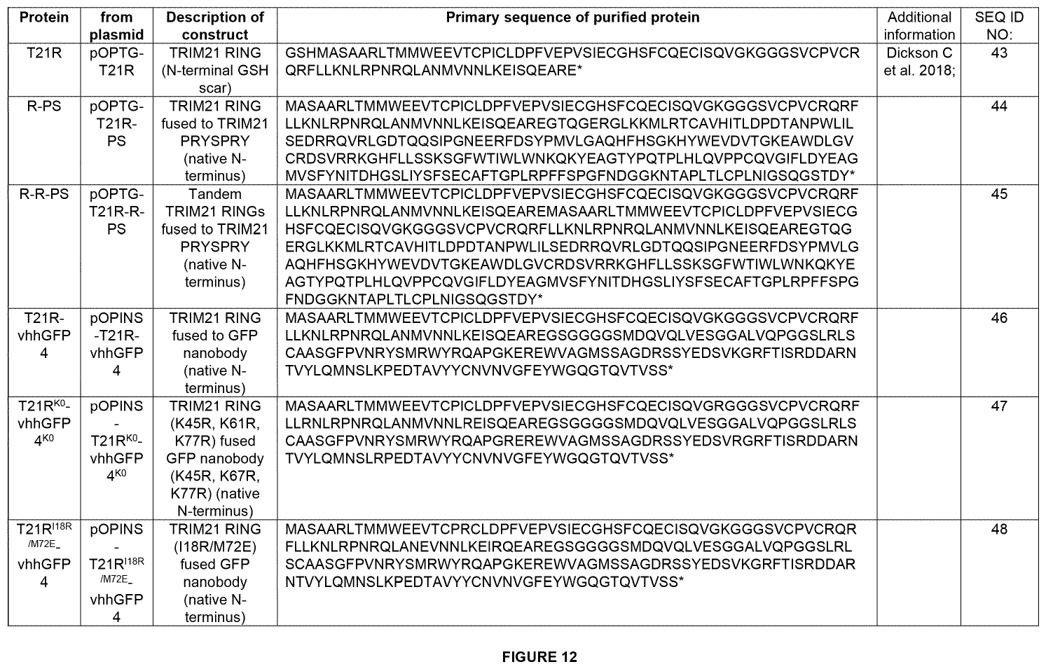

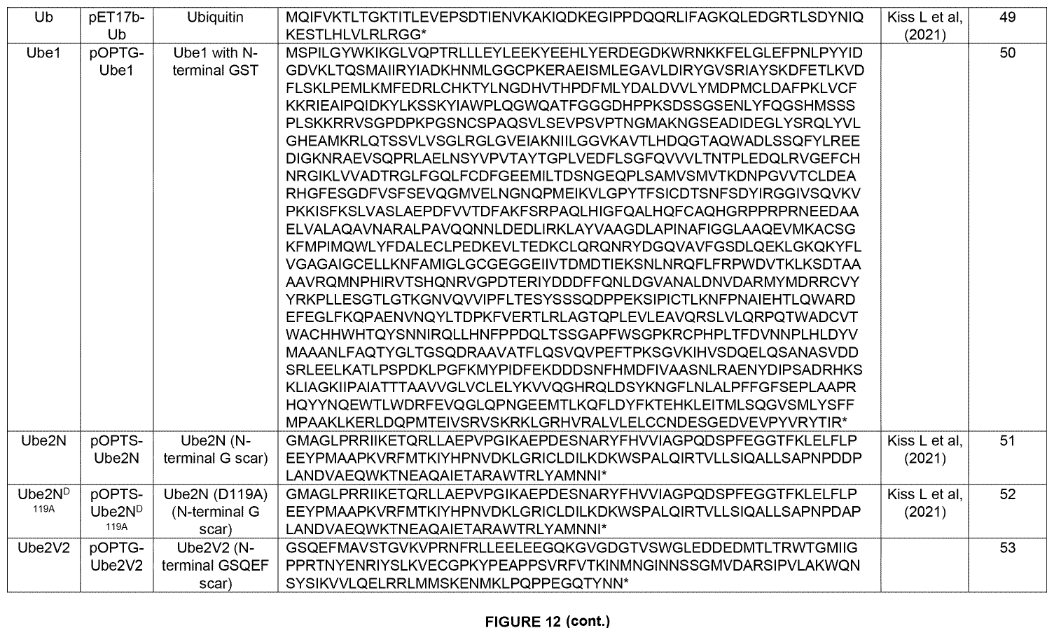

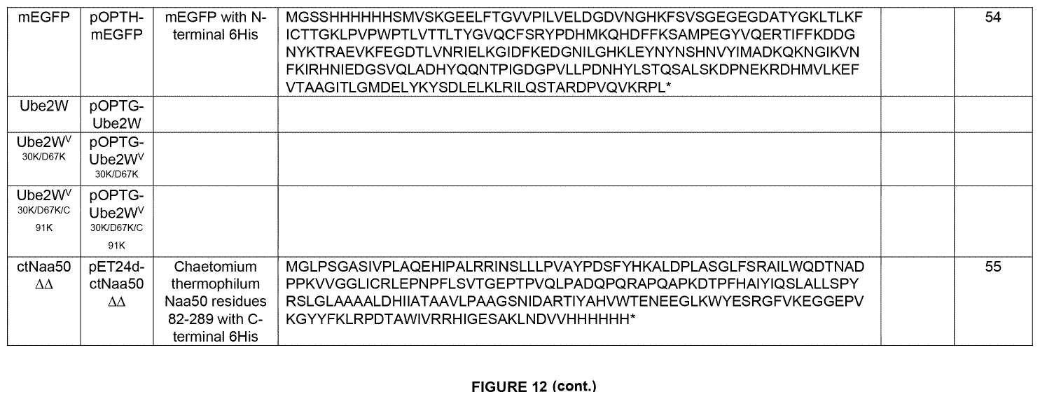

A full list of purified proteins used in this study can be found in (FIG. 12). Ube2W, Ube2N and Ube2V2, and TRIM21 R, R-PS, R-R-PS, T21R-vhhGFP4 and mEGFP were expressed in Escherichia coli BL21 DE3. Ubiquitin and Ube1 were expressed in E. coli Rosetta 2 DE3 cells as previously described (Kiss et al., 2021). Cells were grown at 37° C. and 220 rpm until an OD⁶⁰⁰ of ˜0.7. After induction, the temperature was reduced to 18° C. overnight. For TRIM21 and E2s induction was performed with 0.5 mM IPTG and 10 μM ZnCl₂, for ubiquitin and Ube1 with 0.2 mM IPTG. mEGFP was expressed in ZY autoinduction media (Studier, 2005) at 37° C. and 220 rpm. At OD⁶⁰⁰ of 0.7, the temperature was reduced to 18° C. for expression overnight. After centrifugation, cells were resuspended in 50 mM Tris pH 8.0, 150 mM NaCl, 10 μM ZnCl₂, 1 mM DTT, 20% Bugbuster (Novagen) and Complete protease inhibitors (Roche, Switzerland). For His-tagged proteins, 20 mM imidazole was added to the buffer. Lysis was performed by sonication. TRIM21-R-PS and -R-R-PS were expressed with N-terminal GST-SUMO tag and TRIM-R, Ube2W, Ube2V2 and Ube1 were expressed with N-terminal GST-tag followed by a TEV protease cleavage site and purified via glutathione sepharose resin (GE Healthcare) equilibrated in 50 mM Tris pH 8.0, 150 mM NaCl and 1 mM DTT. The tag was cleaved on beads overnight at 4° C. (with SUMO or TEV protease, respectively). Cleavage with SUMO protease resulted in no cleavage scar on TRIM21-R-PS and -R-R-PS. TEV cleavage results in an N-terminal GSH-scar on TRIM21-R, an N-terminal G-scar on Ube2N, an N-terminal GSQEF-scar on Ube2V2 and an N-terminal GSH-scar on Ube2W. In the case of Ube1, no protease cleavage was performed and the GST-Ube1 fusion protein was eluted using 50 mM Tris pH 8.0, 150 mM NaCl, 10 mM reduced glutathione and 1 mM DTT. mEGFP was expressed with an N-terminal His-tag without protease cleavage site and Ube2N was expressed with an N-terminal His-tag followed by a TEV protease cleavage site. TRIM21 R-vhhGFP4 was expressed as a His-SUMO fusion protein, to generate the native TRIM21 N-terminus after SUMO protease cleavage during purification. His-tagged proteins were purified via Ni-NTA resin equilibrated in 50 mM Tris pH 8.0, 150 mM NaCl, 20 mM imidazole and 1 mM DTT. Proteins were eluted in 50 mM Tris pH 8.0, 150 mM NaCl, 1 mM DTT, and 300 mM imidazole. For Ube2N, TEV-cleavage of the His-tag was performed overnight by dialyzing the sample against 50 mM Tris pH 8.0, 150 mM NaCl, 1 mM DTT, and 20 mM imidazole. Afterward, His-tagged TEV protease was removed by Ni-NTA resin. For T21R-vhhGFP4, SUMO protease cleavage was performed on the Ni-NTA resin overnight at 4° C. Elution was performed on the next day using the equilibration buffer. Finally, size-exclusion chromatography of all proteins was carried out on either HiLoad 26/60 or 16/600 Superdex 75 prep grade column (GE Healthcare) in 20 mM Tris pH 8.0, 150 mM NaCl, and 1 mM DTT, except for GST-Ube1 for which either HiLoad 26/60 or 16/600 Superdex 200 prep grade column (GE Healthcare) were used. Ubiquitin purification was performed following the protocol established by the Pickart lab (Pickart and Raasi, 2005). After cell lysis by sonication (lysis buffer: 50 mM Tris pH 7.4, 1 mg mL-1 Lysozyme (by Sigma Aldrich, St. Louis, USA), 0.1 mg mL-1 DNAse (by Sigma Aldrich, St. Louis, USA), a total concentration of 0.5% perchloric acid was added to the stirring lysate at 4° C. The (milky) lysate was incubated for another 30 min on a stirrer at 4° C. to complete precipitation. Next, the lysate was centrifuged (19,500 rpm) for 30 min at 4° C. The supernatant was dyalized overnight (3,500 MWCO) against 3 L 50 mM sodium acetate pH 4.5. Afterwards, Ub was purified via cation-exchange chromatography using a 20 mL SP column (GE Healthcare) using a NaCl gradient (0-1000 mM NaCl in 50 mM NaAc pH 4.5). Finally, size exclusion chromatography was carried out on a HiLoad 26/60 Superdex 75 prep grade column (GE Healthcare) in 20 mM Tris pH 7.4. Isotopically labelled proteins were expressed using Escherichia coli BL21 DE3 cells in M9 minimal media supplemented with ¹⁵NH₄Cl (Sigma-Aldrich ISOTEC). Chaetomium thermophilum Naa50⁸²⁻²⁸⁹ (Naa50ΔΔ) containing a C-terminal His-tag was expressed using E. coli Rosetta 2 cells in ZY autoinduction media (Studier, 2005) which was grown at 37° C. and 220 rpm. At OD⁶⁰⁰ of 0.7, the temperature was reduced to 18° C. for expression overnight. CtNaa50⁸²⁻²⁸⁹ was purified as follows: Cells were harvested, resuspended in buffer A500 (20 mM HEPES pH 7.5, 500 mM NaCl, 20 mM imidazole) supplemented with a protease inhibitor mix (SERVA Electrophoresis GmbH, Germany) and lysed with a microfluidizer (M1-10L, Microfluidics). The lysate was cleared for 30 min at 50,000 g, 4° C. and filtered through a 0.45 μm membrane. The supernatant was applied to a 1 mL HisTrap FF column (GE Healthcare) for Ni-IMAC (immobilized metal affinity chromatography) purification. The column was washed with buffer A500 and the proteins were eluted with buffer A500 supplemented with 250 mM imidazole. CtNaa5082-289 was subsequently purified by SEC (size-exclusion chromatography) using a Superdex 75 26/60 gel filtration column (GE Healthcare) in buffer G500 (20 mM HEPES pH 7.5, 500 mM NaCl). SUMO protease (MBP-Ulp1 (based on R3 sequence (Lau et al., 2018)) was purified using an MBPTrap HP 5 ml column and eluted with 50 mM Tris pH 8, 150 mM NaCl, 1 mM DTT and 10 mM Maltose. Finally, the eluted fractions were separated on a HiLoad 26/60 Superdex 75 pg SEC column (150 mM NaCl, 50 mM Tris pH 8 and 1 mM DTT).

### Cell Culture

HEK293T (ATCC) and NIH3T3-CAV1-EGFP (Shvets et al., 2015) cells were cultured in DMEM medium (Gibco; 31966021) supplemented with 10% calf serum and penicillin-streptomycin. RPE-1 cells (ATCC) were cultured in DMEM/F-12 medium (Gibco; 10565018) supplemented with 10% Calf Serum and penicillin-streptomycin. All cells were grown at 37° C. in a 5% CO₂ humidified atmosphere and regularly checked to be mycoplasma-free. The sex of NIH3T3 cells is male. The sex of HEK293T and RPE-1 cells is female. For proteasome inhibition experiments, MG132 (Sigma; C2211) was used at a final concentration of 25 μM and Epoxomicin (Sigma; 324801) was used at 10 μM. Following electroporation, cells were grown in medium supplemented with 10% calf serum without antibiotics. Live imaging was performed using the IncuCyte S3 live cell analysis system (Sartorius) housed within a 37° C., 5% CO₂ humidified incubator. For live imaging with the IncuCyte, cell culture medium was replaced with Fluorobrite (Gibco; A1896701) supplemented with 10% calf serum and GlutaMAX (Gibco; 35050061).

### Cell Lines

Cell lines used and generated in this manuscript are detailed in (Table 1). RPE-1 TRIM21 KO cells (Zeng et al., 2021), HEK293T TRIM21 KO cells (Zeng et al., 2019) and NIH3T3-CAV1-EGFP (Shvets et al., 2015) were described previously. For stable expression of CAV1-mEGFP and CAV1-mEGFP-Halo, RPE-1 cells were transduced with lentiviral particles at multiplicity ˜0.1 transducing units per cell and the GFP-positive population selected by flow cytometry. For stable expression of TRIM21-HA at endogenous levels, RPE-1 TRIM21 KO cells were reconstituted with TRIM21-HA construct under control of the native TRIM21 promoter as described previously (Zeng et al., 2019).

### Electroporation

Electroporation was performed using the Neon® Transfection System (Thermo Fisher). Cells were washed with PBS and resuspended in Buffer R at a concentration of between 1-8×10⁷ cells ml⁻¹. For each electroporation reaction 1-8×10⁵ cells in a 10.5 μl volume were mixed with 2 μl of antibody (typically 0.5 mg/ml) or mRNA (typically 0.5 μM) or protein to be delivered. The mixture was taken up into a 10 μl Neon® Pipette Tip, electroporated at 1400V, 20 ms, 2 pulses and transferred to media without antibiotics.

### Measurement of Fluorescence in Live Cells

To quantify GFP fluorescence in live cells, images were acquired and analysed using the IncuCyte live cell analysis system (Sartorius). Within the IncuCyte software, the integrated density (the product of the area and mean intensity) for GFP fluorescence was normalised to total cell area (phase) for each image. Values were normalized to internal controls within each experiment.

### Antibodies

Antibodies and concentrations used for traditional immunoblotting (IB), capillary-based immunoblotting (Jess) and electroporation (EP) are detailed in (Table 2). All antibodies used for electroporation were either purchased in azide-free formats or passed through Amicon Ultra-0.5 100 KDa centrifugal filter devices (Millipore) to remove traces of azide and replace buffer with PBS.

### Adv5 Neutralisation Assay

Adenovirus serotype 5 2.6-del CMV-eGFP (Adv5-GFP, Viraquest) was diluted to 1.1×109 T.U./mL in PBS, and 16 uL was incubated 1:1 with the anti-hexon recombinant humanised IgG1 9C12 or 9C12H433A (Foss et al., 2016) at indicated concentrations, or PBS. After 1 hour incubation at room temperature, complexes were diluted with 250 μL Fluorobrite media and used for Adv5 neutralisation assays. For infections, 4×106 HEK293T TRIM21 KO cells were electroporated with PBS or R-R-PS±N-acetylation and resuspended in 2 mL Fluorobrite media. 50 μL of each cell suspension was combined 1:1 with Adv5:9C12 or Adv5:9C12H433A complexes (for immediate infections) or Fluorobrite (for delayed infections) in 96-well plates. For delayed infections, electroporated cells were allowed to adhere to the plate for 2 hours, then media was replaced with 50 μL of Fluorobrite and infected with 50 μL Ad5:9C12 complexes. Infection levels were quantified using the IncuCyte system by measuring GFP fluorescence area relative to total cell area 16 h post-infection. Infection levels are plotted relative to Adv5-GFP infection the absence of 9C12 antibody.

### NFKB Signalling Assay

HEK293T TRIM21 KO cells were transfected with 2 ug pGL4.32 NF-KB Luciferase plasmid (Promega), using 12 μL of Viafect (Promega) in 200 uL OptiMEM (Thermo Fisher). Twenty-four hours later 4×106 transfected cells were electroporated with PBS or R-R-PS±N-acetylation and resuspended in 1 mL DMEM media. For infections, Adv5:9C12 complexes were prepared as described above, except Ad5-GFP was diluted to 1.1×1010, 9C12 was used at 20 μg/mL, and the complex was diluted into 150 μL DMEM after 1 hour incubation. 50 μL of the electroporated cell suspension was mixed 1:1 with Adv5:9C12 complexes or PBS (control), then lysed 4 hours later in 100 μL of SteadyLite Plus luciferase reporter (PerkinElmer). As an internal control, TNF-α was used at 10 ng/uL. Luminance was recorded on a PheraStar FS (BMG LabTech).

### Immunoblotting

Samples were run on NuPAGE 4-12% Bis-Tris gels (ThermoFisher) and transferred onto nitrocellulose membrane. Membranes were incubated in blocking buffer (PBS, 0.1% Tween20, 5% milk) for 1 h at room temp prior to incubation with antibodies. Antibodies and dilutions (in blocking buffer) used for immunoblotting (IB) are detailed in (Table 2). HRP-coupled antibodies were detected by enhanced chemiluminescence (Amersham, GE Healthcare) and X-ray films. IRDye-coupled antibodies were detected using LI-COR Odyssey CLx imaging system.

### Capillary-Based Immunoblotting

RIPA buffer protein extracts were diluted 1:2 in 0.1× sample buffer (bio-techne; 042-195) and run on the Jess Simple Western system using a 12-230 kDa separation module (bio-techne) according to manufacturer's instructions. Antibodies and dilutions used for capillary-based immunoblotting (Jess) are detailed in (Table 2). Protein peak areas were quantified using Compass software (bio-techne) and normalized to internal protein loading controls within each capillary.

### In Vitro Ubiquitination Assays

Ube2W-dependent TRIM21-mono-ubiquitination assays were performed in 50 mM Tris pH 7.4, 150 mM NaCl, 2.5 mM MgCl2 and 0.5 mM DTT. The reaction components were 2 mM ATP, 1 μM GST-Ube1, 80 μM ubiquitin and the indicated concentrations of Ube2W and TRIM21, respectively. The reaction was stopped by addition of LDS sample buffer containing 50 mM DTT at 4 0C. Next, samples were boiled at 90° C. for 2 min. For reactions using 10 μM TRIM21, visualization was performed by Instant Blue stained LDS-PAGE only. Polyubiquitin chain extension assays were performed as above, but instead of Ube2W, 0.5 μM Ube2N/Ube2V2 were added. For antibody-induced mono-ubiquitination similar conditions were used as for the LDS-PAGE analysed mono-ubiquitination described above. However, the concentration of TRIM21 was reduced to 100 nM and GST-Ube1 to 0.25 μM. Anti-GFP antibody (9F9.F9) was added in one molar equivalent to TRIM21. The reaction was initiated by addition of Ube2W (0, 50, 100, 200 nM). The reaction was stopped by addition of LDS sample buffer at 4° C. Samples were boiled at 90° C. for 2 min and resolved by LDS-PAGE. TRIM21 was visualized using western blot. In vitro reconstitution of Trim-Away ubiquitination events was performed similar to the antibody-induced mono-ubiquitination experiments described above. E2 concentrations were 200 nM Ube2W and 0.5 μM Ube2N/Ube2V2 and His-mEGFP was used as Trim-Away target at 200 nM.

### Acetylation and Monoubiquitination Assay

N-terminal acetylation of TRIM21 was mediated by the Chaetomium thermophilum N-acetyl transferase (NAT) Naa50AA. Acetylation reactions were performed in 50 mM Tris pH 7.4 and 150 mM NaCl for 4 h at 25° C. The reactions contained 20 μM TRIM21, 1 mM Acetyl-CoA and 1 μM CtNaa50ΔΔ. After the Acetylation reaction was finished, it was mixed 1:1 with a Ube2W-ubiquitination mix containing 100 mM Tris pH 7.4, 300 mM NaCl, 5 mM MgCl2 and 1 mM DTT, 4 mM ATP, 2 μM GST-Ube1, 160 μM ubiquitin and 2 μM Ube2W. The Ube2W ubiquitinaton reaction was performed for 1 h at 37° C. and stopped by addition of LDS sample buffer containing 50 mM DTT at 4° C., followed by boiling the samples at 90° C. for 2 min. Visualization was performed by Instant Blue stained LDS-PAGE only.

### NMR Spectroscopy

Two-dimensional NMR measurements (¹⁵N-HSQC) were performed at 25° C. on Bruker Avance 1 600 MHz spectrometer equipped with 5 mm ¹H-¹³C-¹⁵N cryogenic probe. Data was processed with the program Topspin (Bruker BioSpin GmbH, Germany) and analyzed with the program CCPN analysis v2(Vranken et al., 2005). Samples were buffer exchanged into 50 mM deuterated Tris pH 7.0, 150 mM NaCl and 1 mM deuterated DTT (Cambridge Isotopes, United Kingdom).

Chemical shift perturbations (CSPs) were calculated using the equation (3):

$$Δ {δ}_{N , HN} = \sqrt{{( Δδ {(}^{1} H )}^{2} + {( Δ δ {(}^{15} N )}^{2} * 0.14 )} \qquad ( 3 )$$

where Δδ_{N, HN} is the CSP, Δδ(¹H) and Δδ(¹⁵N) are the chemical shift differences between the position of proton or nitrogen signal in absence and presence of titrant. TRIM21 assignments were used from a previous publication (Dickson et al., 2018).

### Mass Spectrometry

Excised protein gel pieces were destained with 50% v/v acetonitrile: 50 mM ammonium bicarbonate. After reduction with 10 mM DTT and alkylation with 55 mM iodoacetamide, the proteins were digested overnight at 37° C. with 6 ng μL⁻¹ of Asp-N(Promega, UK). Peptides were extracted in 2% v/v formic acid: 2% v/v acetonitrile and subsequently analyzed by nano-scale capillary LC-MS/MS with an Ultimate U3000 HPLC (Thermo Scientific Dionex, San Jose, USA) set to a flowrate of 300 nL min⁻¹. Peptides were trapped on a C18 Acclaim PepMap100 5 μm, 100 μm×20 mm nanoViper (Thermo Scientific Dionex, San Jose, USA) prior to separation on a C18 T3 1.8 μm, 75 μm×250 mm nanoEase column (Waters, Manchester, UK). A gradient of acetonitrile eluted the peptides, and the analytical column outlet was directly interfaced using a nano-flow electrospray ionization source, with a quadrupole Orbitrap mass spectrometer (Q-Exactive HFX, ThermoScientific, USA). For data-dependent analysis a resolution of 60,000 for the full MS spectrum was used, followed by twelve MS/MS. MS spectra were collected over a m/z range of 300-1,800. The resultant LC-MS/MS spectra were searched against a protein database (UniProt KB) using the Mascot search engine program. Database search parameters were restricted to a precursor ion tolerance of 5 ppm with a fragmented ion tolerance of 0.1 Da. Multiple modifications were set in the search parameters: two missed enzyme cleavages, variable modifications for methionine oxidation, cysteine carbamidomethylation, pyroglutamic acid and protein N-term acetylation. The proteomics software Scaffold 4 was used to visualize the fragmented spectra.

### Statistical Analysis

Average (mean), standard deviation (s.d.), standard error of the mean (s.e.m) and statistical significance based on Student's t-test (two-tailed) and one- or two-way ANOVA were calculated in Microsoft Excel or Graphpad Prism. Significance are represented with labels ns (not significant, P>0.05), *(P:0.05), **(P:0.01), ***(P:0.001), ****(P:0.0001).

### Crystallography

Crystals of TRIM21-RING:Ube2W^{V}30K/D^{67K}/C^{91K} complex were grown in 2 n1 drops at 10 mg/ml at 17° C. in 0.1 M Bicine pH 9.0, 5% PEG 6000, 0.1 M TCEP hydrochloride. Diffraction experiments were performed at the European Synchrotron Radiation Facility at beamline ID23 using a Dectris PILATUS 6M detector at a wavelength of 0.984004 Å. The diffraction data at 2.25 Å was processed using XDS. The structure was solved by molecular replacement using Phaser (Adams et al., 2010) with TRIM21 RING domain (5OLM (Dickson et al., 2018)) and Ube2W residues 1-118 (2MT6 (Vittal et al., 2015)) as search models. Model building and real-space-refinement were carried out in coot (Emsley and Cowtan, 2004), and refinement was performed using REFMAC5 and phenix-refine (Afonine et al., 2012). Model and structure factors have been deposited at the PDB with the accession code 8A58.

### Results

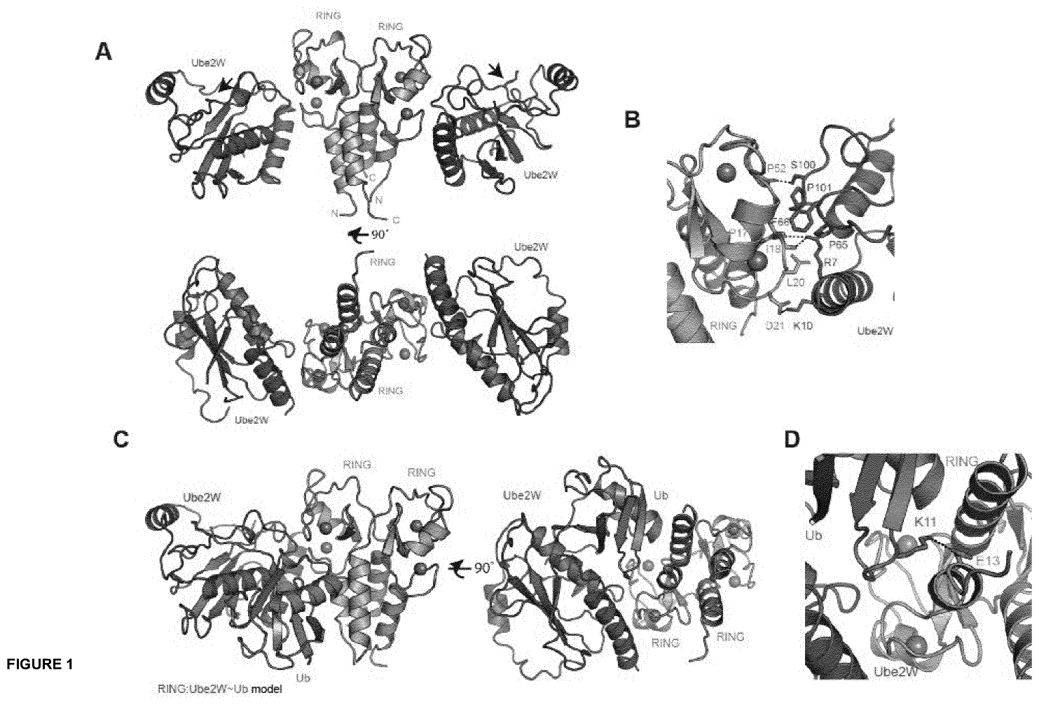

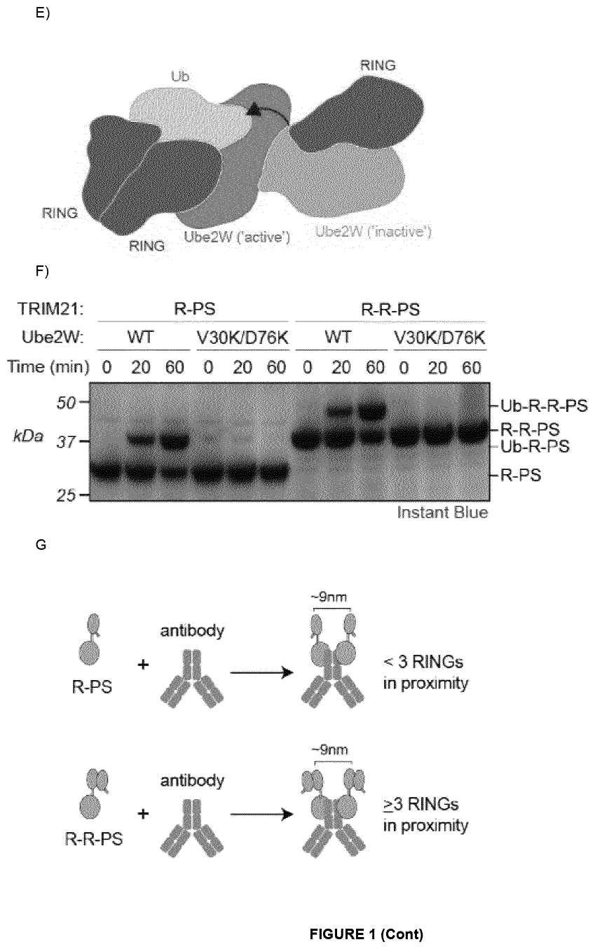

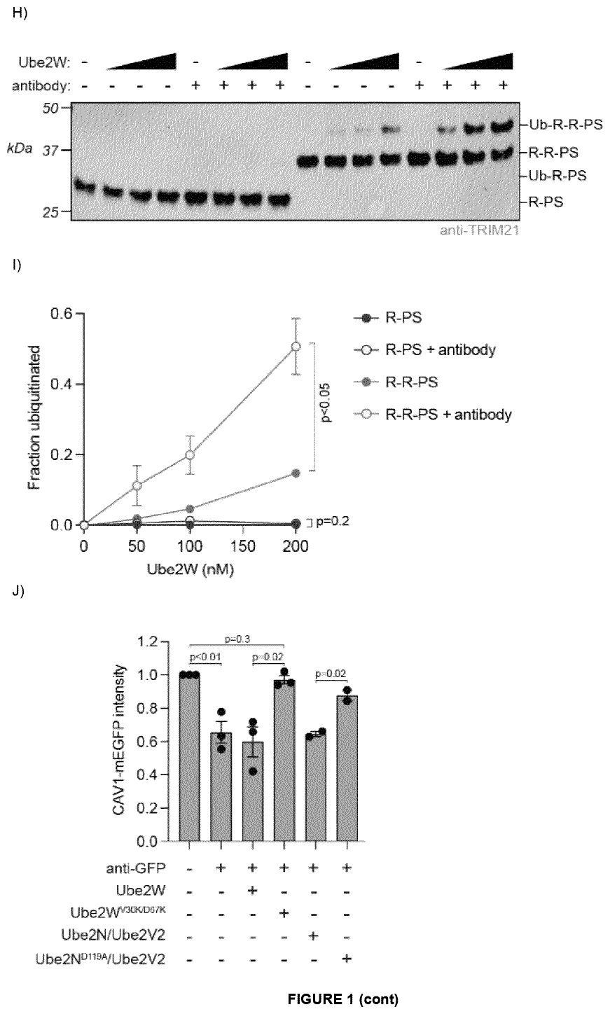

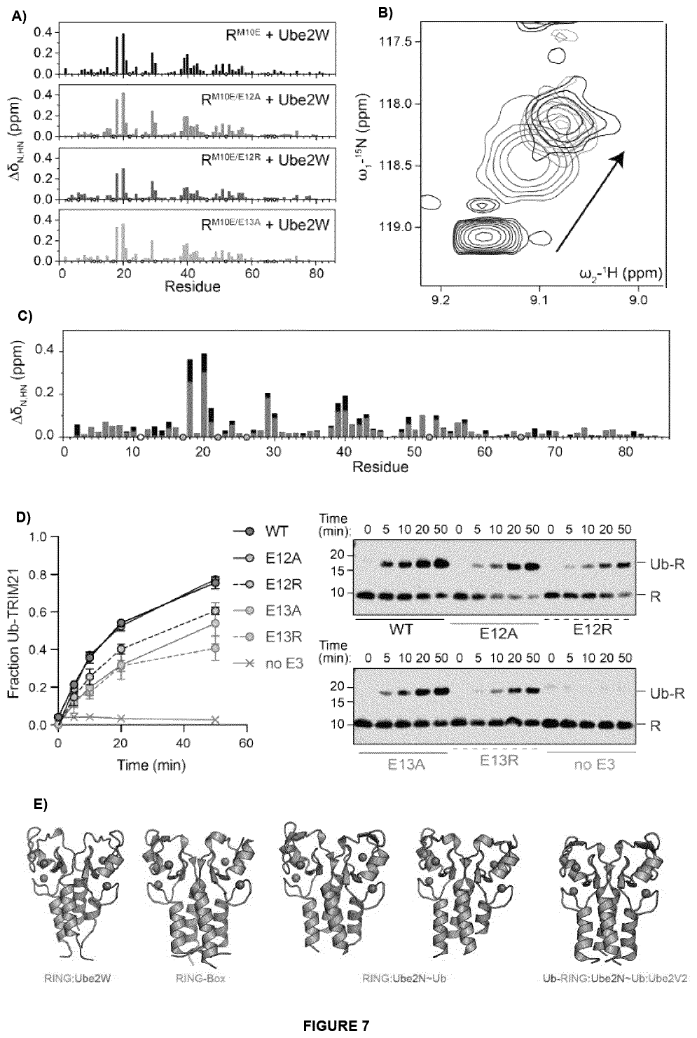

To understand how TRIM21 recruits Ube2W, we solved the crystal structure of the RING domain (R) in complex with a Ube2W dimerization mutant (Vittal et al., 2013b) in which the active site cysteine was also replaced with lysine (Ube2WV30K/D6^{7K}/C⁹1K). Two copies of each Ube2W and RING could be found in the asymmetric unit, with the two RINGs forming a homodimer as described previously (Dickson et al., 2018; Kiss et al., 2021; Kiss et al., 2019) (FIG. 1A). RING and Ube2W engage each other via the canonical RING:E2 interface (FIG. 1B). By superposing the R:Ube2W structure with Ube2N-Ub from the previously determined Ub-R:Ube2N-Ub:Ube2V2 structure (Kiss et al., 2021), the activation of Ube2W˜Ub was modelled (FIG. 1C). Overall, the arrangement of a Ube2W˜Ub is similar to Ube2N˜Ub, when being activated by TRIM21 (Kiss et al., 2021; Kiss et al., 2019). In this model, the donor ubiquitin is in the closed conformation, stabilized by both RING protomers. Interestingly, the model also suggests that TRIM21 E13 might engage ubiquitin K11 to stabilize the closed conformation (FIG. 1D). E13 is part of a tri-ionic motif that was identified to drive Ube2N-Ub interaction (Kiss et al., 2019). We tested whether this motif is involved in Ube2W binding by performing NMR titrations of ¹⁵N-labelled TRIM21 tri-ionic mutants against Ube2W^{V30K/D67K/C91K}. Mutation of the tri-ionic residues E12 and E13 to alanine did not lead to obvious reductions in the observed chemical shift perturbations (CSPs) (FIG. 7A-C). Moreover, tri-ionic mutants had only a modest effect on TRIM21 monoubiquitination (FIG. 7D). TRIM21 mutants E13A and E13R both showed a slight reduction in activity, suggesting that residue E13 could indeed interact with ubiquitin K11, but this is not as critical as for Ube2N-Ub (Kiss et al., 2019). When comparing the RING in the R:Ube2W structure to the apo-(Dickson et al., 2018) and Ube2N-Ub (Kiss et al., 2019) engaged structures, we noted that the N- and C-terminal helices of the RINGs are partly unfolded when bound by Ube2W (FIG. 7E). While this may reflect differences in crystallisation, it could suggest that Ube2W has the potential to destabilize the 4-helix bundle, thereby generating a disordered N-terminus for modification. Even so, this is insufficient to explain how Ube2W can modify the N-terminus of a RING it is bound to, as it is located far away from the E2 active site.

We postulated that as Ube2W is normally dimeric (Vittal et al., 2013a), it may utilise a similar catalytic RING topology to that previously described for the Ube2N/Ube2V2 heterodimer (Kiss et al., 2021). Under such an arrangement, two RINGs could form a dimer to act as the enzyme, activating the donor ubiquitin on one Ube2W monomer, while a third RING acts as the substrate, oriented by the second Ube2W monomer to allow attack on the N-terminus (FIG. 1E). We tested this hypothesis using two TRIM21 constructs, carrying either one (R) or two (R-R) RINGs and a PRYSPRY (PS) domain (R-PS and R-R-PS). Both constructs were efficiently monoubiquitinated by Ube2W (FIG. 1F). However, while Ube2W dimerization was deemed not to be important for activity (Vittal et al., 2013, Cell Biochem Biophys), this activity was abolished when monomeric Ube2W^{V30K/D67K} was used (FIG. 1F). This is consistent with Ube2W dimerization being used to orient one RING domain as a substrate (FIG. 1E). Surprisingly, no difference between R-PS and R-R-PS was observed, probably because the relatively high TRIM21 concentrations were sufficient to drive RING dimerization of R-PS.

Previously we have shown that TRIM21 is activated in cells by substrate-induced clustering (Zeng et al., 2021) and indeed the addition of IgG Fcto in vitro ubiquitination experiments is required to induce TRIM21-mediated K63-chain formation by Ube2N/Ube2V2 under near-physiological enzyme concentrations(Kiss et al., 2021). We therefore reduced R-PS or R-R-PS concentrations and titrated Ube2W either in the presence or absence of IgG. Under these conditions, monoubiquitination was only observed with R-R-PS (FIG. 1G&H). Moreover, R-R-PS monoubiquitination was greatly stimulated by the addition of IgG (FIG. 1H&I). Importantly, we observed that endogenous TRIM21 is similarly dependent upon dimeric Ube2W for substrate degradation in cells. Electroporation of Ube2WV30K/D6^{7K} but not wild-type Ube2W, during a Trim-Away experiment prevented anti-GFP antibody-induced degradation of the substrate CAV1-mEGFP in cells. As a positive control, we also compared wild-type Ube2N/Ube2V2 with a mutant in which the residue responsible for deprotonating K63 on the acceptor ubiquitin is replaced with alanine (Ube2N^{N119A}/Ube2V2) (Kiss et al., 2021). The catalytically inactive mutant failed to degrade CAV1-mEGFP (FIG. 1J). These results confirm previous observations using siRNA that both Ube2W and Ube2N/Ube2V2 are required for TRIM21 activity (Fletcher et al., 2015b; McEwan et al., 2013). Taken together, the cellular and in vitro data suggest that antibody binding promotes TRIM21 RING monoubiquitination by dimeric Ube2W through a similar trans mechanism to Ube2N/Ube2V2 and that this is required for substrate degradation.

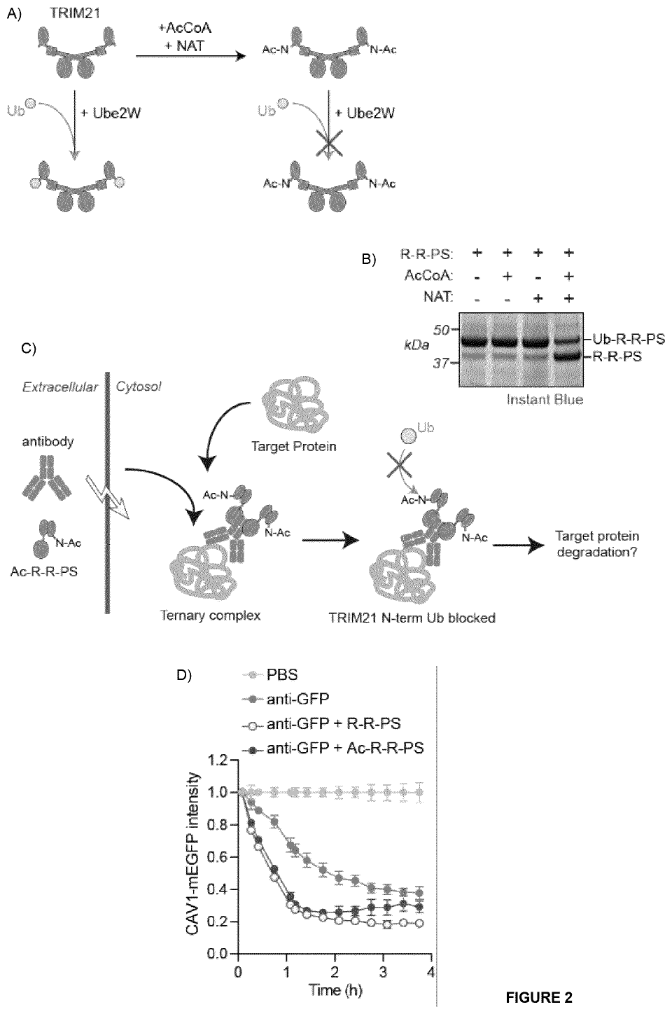

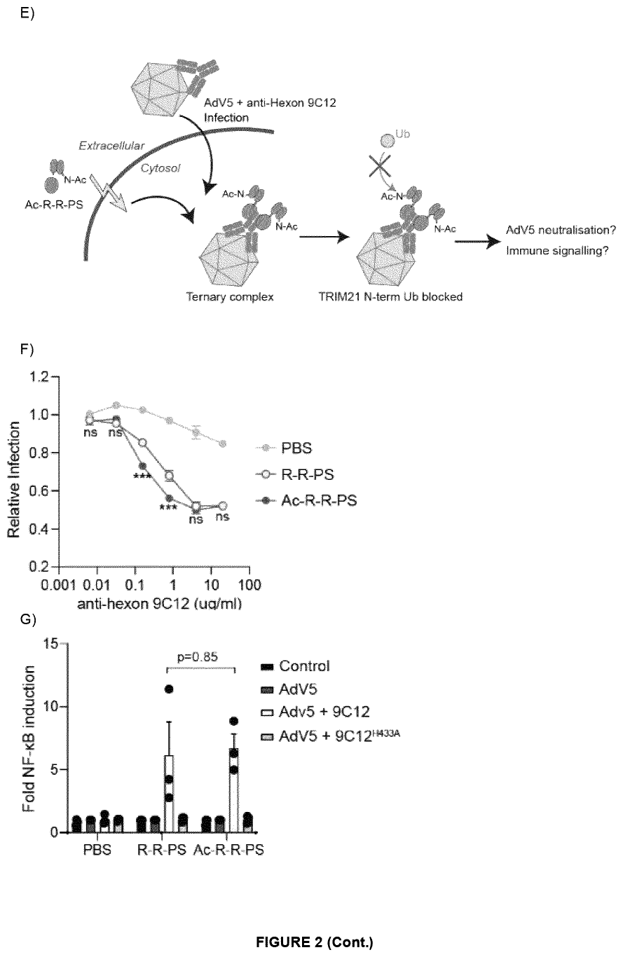

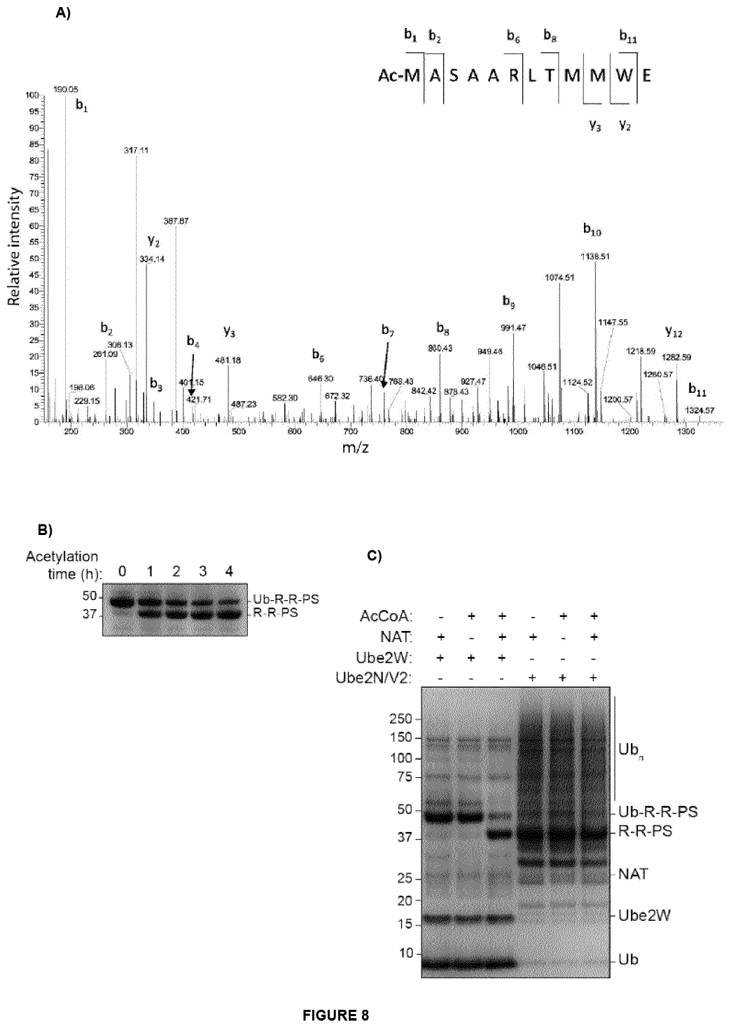

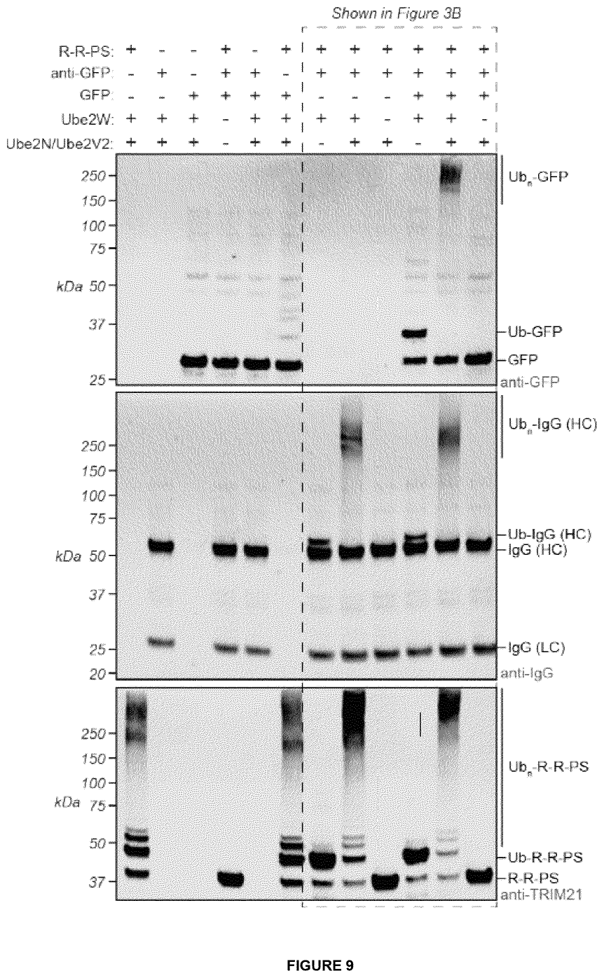

While Ube2W is needed for substrate degradation and TRIM21 can be monoubiquitinated by the E2 in vitro, this does not prove that one requires the other. To investigate the requirement for TRIM21 N-terminal monoubiquitination, we decided to block it biochemically via N-acetylation. N-acetylation is an irreversible modification catalysed in cells by N-Acetyl Transferases (NATs) using the co-factor Acetyl-CoA (Aksnes et al., 2019). We chose a NAT from Chaetomium thermophilum, a thermostable enzyme capable of interacting with human proteins (Weyer et al., 2017), and incubated it with R-R-PS in the presence of Acetyl-CoA. Successful N-terminal acetylation was confirmed by LC-MS/MS (FIG. 8A). N-terminally acetylated R-R-PS (Ac-R-R-PS) was added together with Ube2W and ubiquitin to test whether monoubiquitination was inhibited (FIG. 2A). In the absence of acetylation, all R-R-PS was monoubiquitinated, whereas R-R-PS incubated with NAT and Acetyl-CoA remained predominantly non-ubiquitinated (FIG. 2B). The degree of monoubiquitination inhibition was proportional to the time of NAT and Acetyl-CoA incubation (FIG. 8B). Importantly, the formation of free K63-linked ubiquitin chains was not compromised, demonstrating that the acetylated RING remains catalytically active (FIG. 8C). These results confirm both that TRIM21 RING is monoubiquitinated by Ube2W at its N-terminus and that this can be inhibited by N-terminal acetylation.

With a method to specifically block N-terminal ubiquitination of the RING, we tested whether this is required for substrate degradation. Either R-R-PS or Ac-R-R-PS was electroporated together with anti-GFP antibody into cells expressing CAV1-mEGFP and the kinetics of substrate degradation monitored through fluorescence detection (FIG. 2C). Consistent with previous Trim-Away experiments, electroporation of anti-GFP antibody drove substrate degradation via endogenous TRIM21 (FIG. 2D). However, CAV1-mEGFP degradation was substantially accelerated through the delivery of exogenous R-R-PS. Importantly, ligase acetylation (Ac-R-R-PS) had no impact and degradation proceeded with identical kinetics (FIG. 2D). Next, we tested whether N-terminal acetylation was required for TRIM21 antiviral function. Cells were electroporated with either R-R-PS or Ac-R-R-PS, then infected with Adenovirus 5 (AdV5) encoding the gene for GFP in the presence of the anti-hexon antibody 9C12. Productive infection was monitored by measuring GFP expression 24 hours post-challenge (FIG. 2E). Previous experiments have shown that when TRIM21 binds to antibody-coated virus it blocks infection by mediating proteasomal-degradation of the virion (Mallery et al., 2010). As expected, electroporation of R-R-PS neutralized AdV5 in an antibody-dose dependent manner (FIG. 2F). Ac-R-R-PS was at least as active as R-R-PS at neutralizing AdV5 infection and at intermediate antibody concentrations was more effective. In addition to mediating virion degradation, TRIM21 also activates innate immune signalling upon detection of antibody-coated virus (McEwan et al., 2013). We therefore repeated infection experiments in cells containing an NF-κB promoter-driven luciferase gene. R-R-PS and Ac-R-R-PS were equally effective at stimulating NF-κB-driven transcription in response to antibody-coated Adv5 (FIG. 2G). In both cases, this was dependent upon direct antibody engagement as the 9C12 mutant H433A, which specifically ablates binding to the PRYSPRY (McEwan et al., 2012), failed to activate NF-κB. Taken together, the data show that N-terminal ubiquitination of the RING ligase is dispensible for both Trim-Away or TRIM21 antiviral functions.

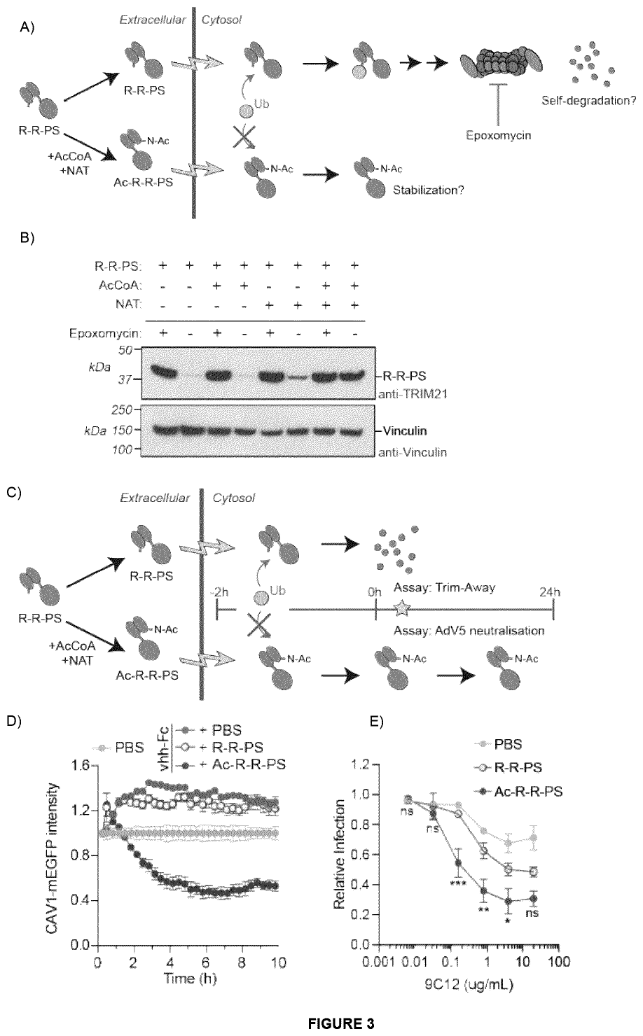

We reasoned that if RING ligase autoubiquitination is not required for substrate degradation, perhaps it is involved in mediating self-turnover. We therefore electroporated acetylated R-R-PS into RPE-1 cells and monitored protein levels after 1 hour (FIG. 3A). Comparison of epoxomicin treated and untreated cells revealed that non-acetylated R-R-PS was readily degraded by the proteasome (FIG. 3B, first two lanes). In contrast, epoxomicin had little impact on Ac-R-R-PS protein levels, indicating that inhibiting N-terminal ubiquitination blocks proteasomal degradation (FIG. 3B, last two lanes). This data suggests that while ligase N-terminal ubiquitination is not required for substrate degradation it may be used to regulate ligase levels. To test this, we repeated our electroporation experiments with R-R-PS or Ac-R-R-PS but measured either Trim-Away or AdV5 neutralization after a delay of several hours (FIG. 3C). For Trim-Away experiments, this was accomplished by co-electroporating the RNA encoding the antibody construct responsible for recruiting R-R-PS to substrate (vhhGFP4-Fc). Trim-Away is thus delayed by several hours while vhhGFP4-Fc is expressed. Under this experimental regime, there was no longer any substrate degradation in cells electroporated with R-R-PS (FIG. 3D, compare with FIG. 2D). In contrast, Ac-R-R-PS degraded CAV1-mEGFP just as efficiently as when Trim-Away proceeds immediately upon ligase electroporation (FIG. 3D). For AdV5 neutralization experiments, cells were infected 2 hours post-ligase delivery. In this case, neutralisation by R-R-PS neutralization was severely attenuated with Ac-R-R-PS inhibiting infection significantly more efficiently (FIG. 3E).

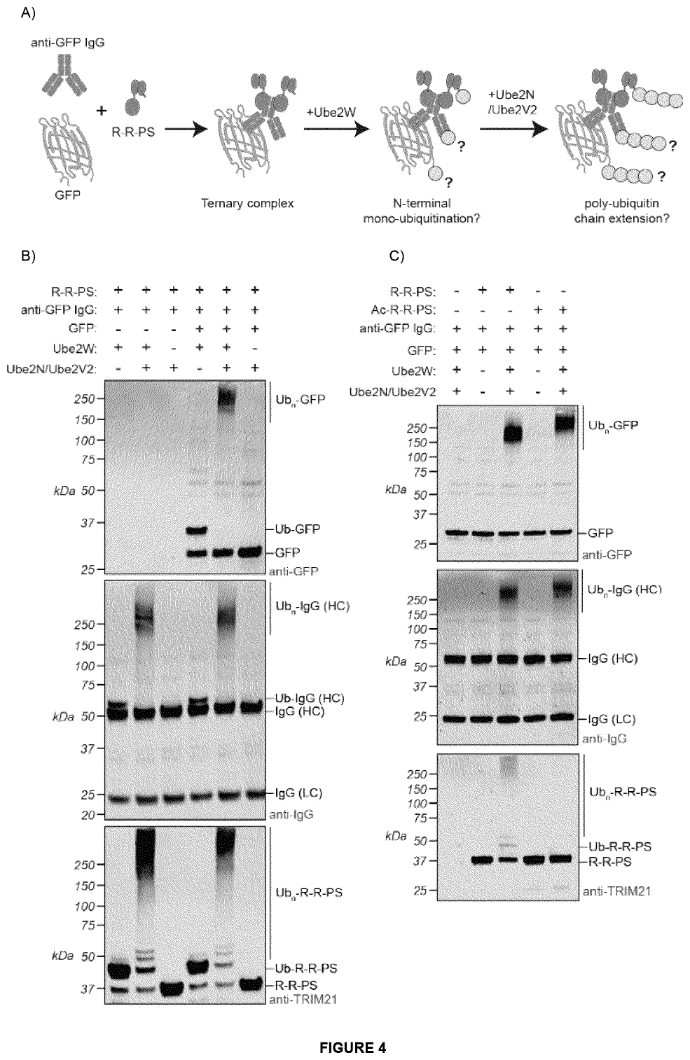

As ligase autoubiquitination is not required for substrate degradation, we investigated whether this might be driven by substrate ubiquitination instead. To test this, we performed in vitro ubiquitination experiments with R-R-PS, anti-GFP antibody and recombinant mEGFP substrate (FIG. 4A). We observed simultaneous ubiquitination of R-R-PS, antibody heavy chain and mEGFP (FIGS. 4A and B). Monoubiquitination was observed upon incubation with Ube2W alone, whereas in conditions where Ube2N/V2 was also included this resulted in anchored polyubiquitin chains. Importantly, substrate ubiquitination only occurred when both R-R-PS and antibody were present (FIG. 9). This is consistent with the requirement for antibody to recruit the TRIM21 ligase to its substrate. Next, we asked whether substrate ubiquitination occurs independently of ligase ubiquitination or if the latter modification is required to stimulate catalytic activity. To do this, we repeated our in vitro ubiquitination assay in the presence of both E2s and compared R-R-PS with Ac-R-R-PS. Ligase acetylation blocked autoubiquitination but did not interfere with polyubiquitination of either antibody heavy chain or substrate (FIG. 4C). This data shows that TRIM21 catalyses polyubiquitination of an antibody-coated substrate and that this can occur independently of ligase autoubiquitination. Nevertheless, the fact that TRIM21, antibody and substrate can be simultaneously polyubiquitinated in vitro is consistent with in-cell Trim-Away data showing that all three components are simultaneously degraded (Clift et al., 2017).

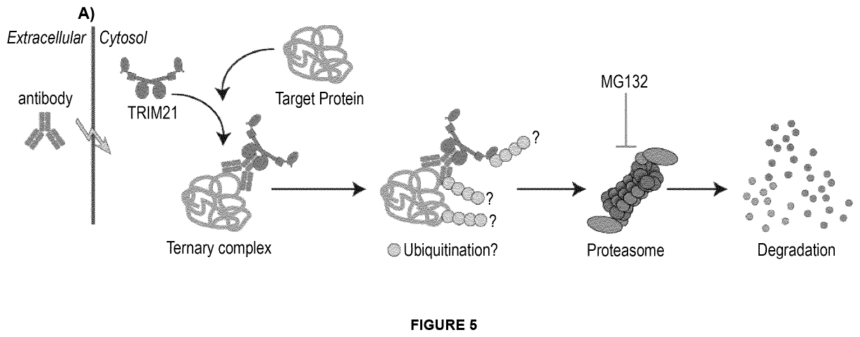

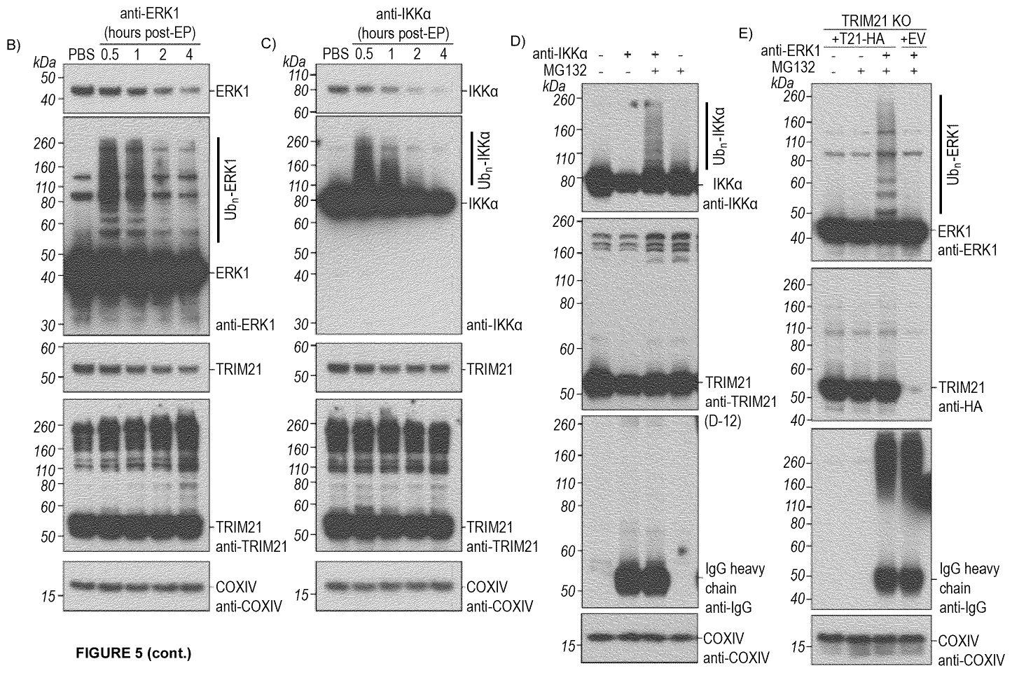

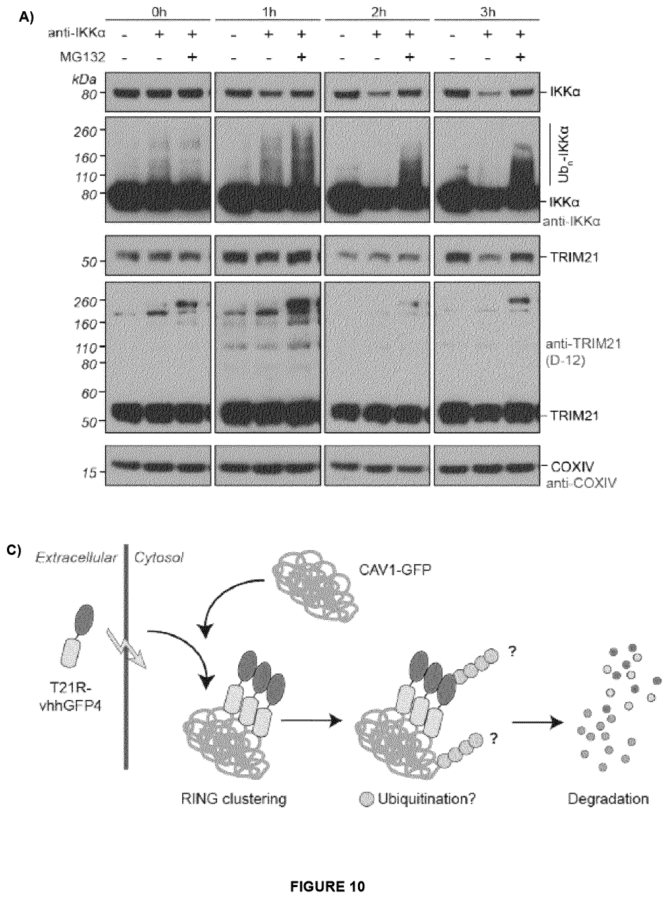

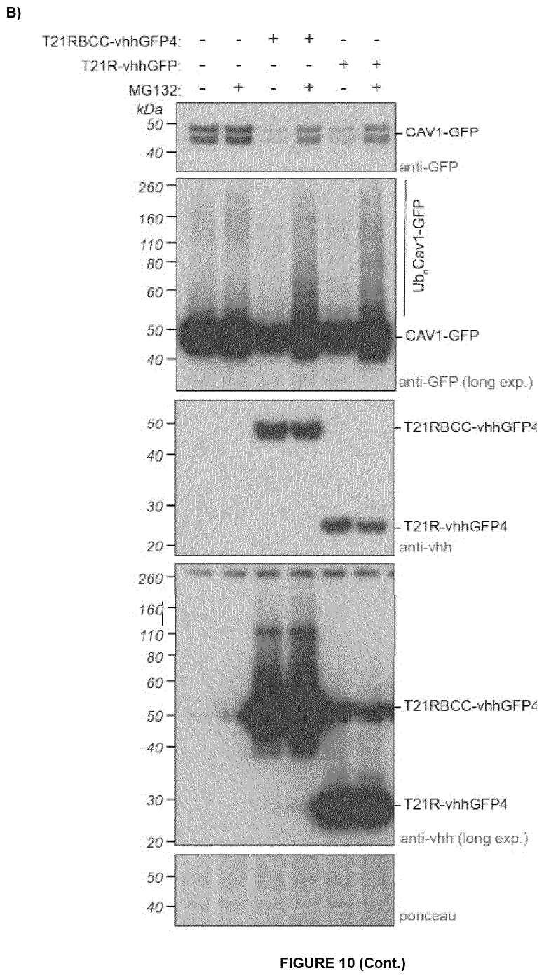

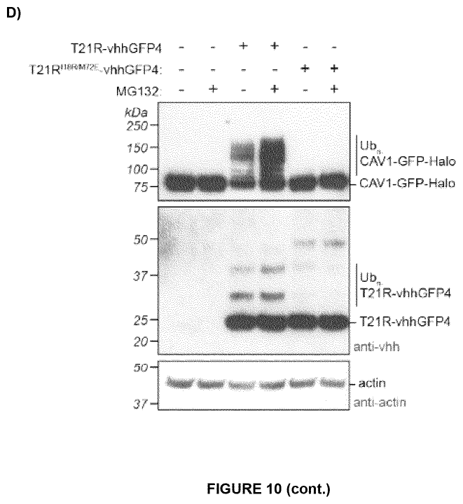

Next, we attempted to monitor substrate ubiquitination during Trim-away degradation in living cells (FIG. 5A). We chose two kinase substrates, ERK1 and IKKα, and blotted for protein levels at various timepoints post antibody electroporation. For both substrates, high-molecular weight bands or smearing consistent with polyubiquitination was observed at 30 minutes post-electroporation (FIG. 5B&C). These high molecular weight bands decreased over the next few hours simultaneous with a reduction in substrate protein levels. To obtain further evidence for substrate polyubiquitination we repeated the IKKα Trim-Away experiment in the presence of proteasome inhibitor MG132. Addition of MG132 had no impact in control cells, but in the presence of electroporated antibody higher molecular weight laddering was clearly observed (FIG. 5D). When performed as a time-course experiment, this revealed that IKKα ubiquitinated species first formed and then was depleted coincident with protein degradation. Treatment with MG132 both blocked degradation and led to a steady accumulation of ubiquitinated material (FIG. 10A). We also probed for TRIM21 and observed a decrease in protein levels that also paralleled substrate depletion (FIGS. 5B and C). Higher molecular weight bands were observed for TRIM21 that may also indicate polyubiquitination, however whilst these increased during ERK1 Trim-Away there was no change with IKKα. To test whether antibody-dependent substrate ubiquitination is mediated by TRIM21 we repeated our experiments in TRIM21 knockout cells reconstituted with either HA-tagged TRIM21 or an empty vector control. Antibody-induced ERK1 laddering indicative of polyubiquitination was observed knockout cells reconstituted with TRIM21-HA but not empty vector (FIG. 5E). These data indicate that substrates are ubiquitinated during Trim-Away in live cells in an antibody- and TRIM21-dependent manner.

Previously, we have shown that Trim-Away can be performed in the absence of antibody by fusing a substrate-targeting nanobody directly to domains from TRIM21 (Zeng et al., 2021). We used this approach to determine whether there is something particular to the ternary complex formed between TRIM21:antibody:substrate that is required for substrate ubiquitination. We tested two fusion constructs in which either the TRIM21 RING, B Box and coiled-coil (T21RBCC-) or RING domain alone (T21R-) is fused to the anti-GFP nanobody (vhhGFP4). The fusion constructs were transduced as RNA into cells expressing CAV1-mEGFP and both ubiquitination and degradation was monitored. Efficient Trim-Away was observed using both fusion constructs (FIG. 10C). Treatment with MG132 inhibited degradation and led to a coincident accumulation of ubiquitinated substrate (FIG. 10C). These results show that neither antibody nor a ternary complex is required for TRIM21-mediated substrate ubiquitination and degradation. Introducing two RING-inactivating mutations into the T21R-vhhGFP4 construct (T21R^{I18R/M72E}-vhhGFP4) (Zeng et al., 2021) completely abolished both substrate ubiquitination and degradation, suggesting these processes are driven by TRIM21 RING catalytic activity.

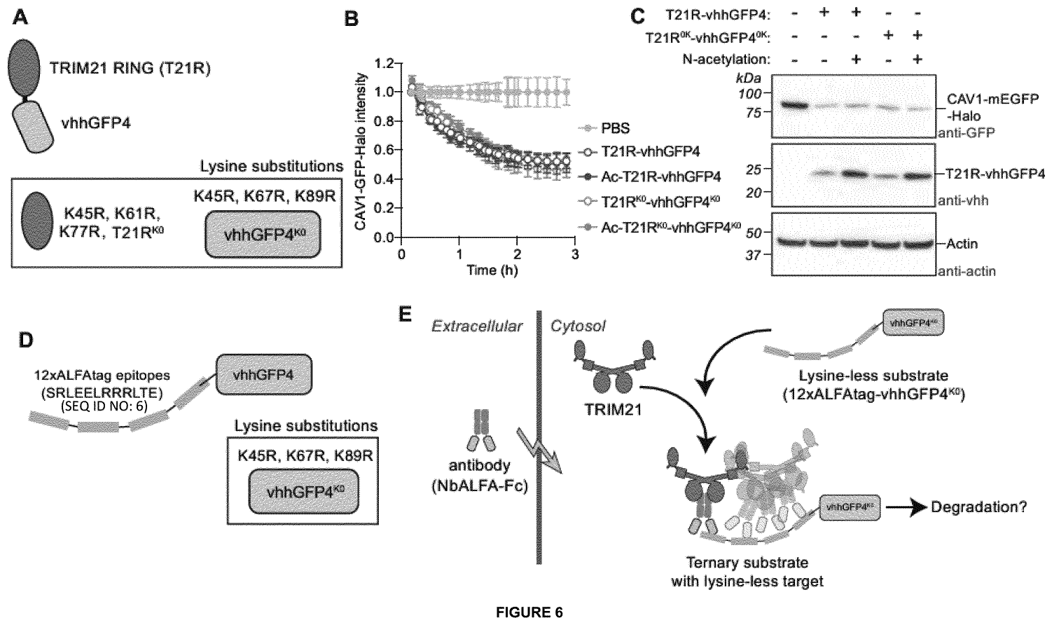

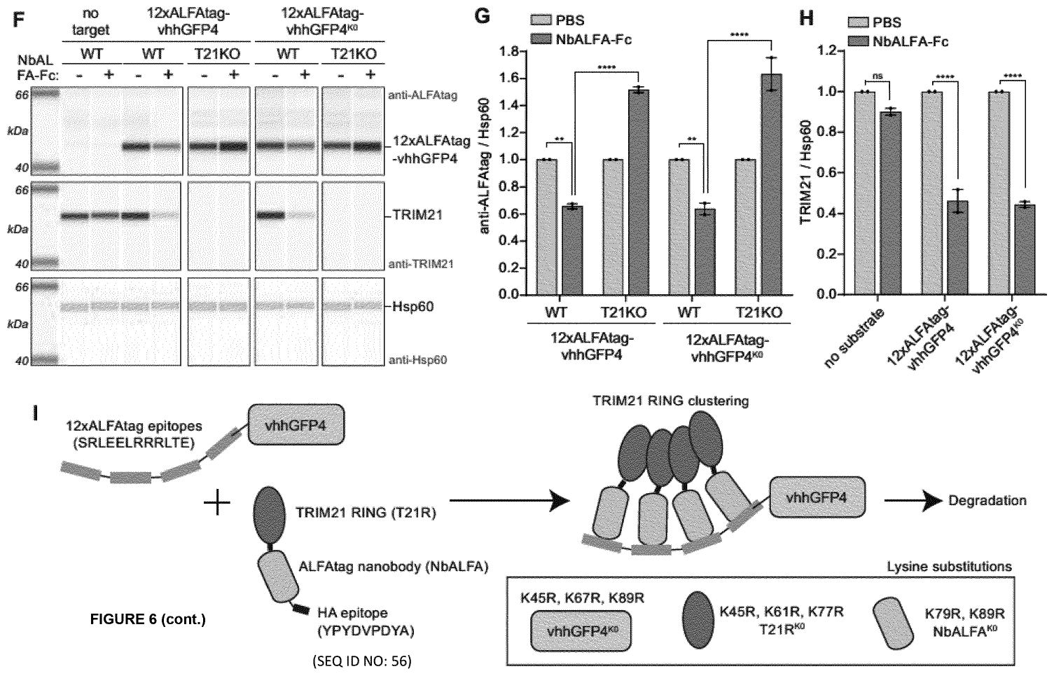

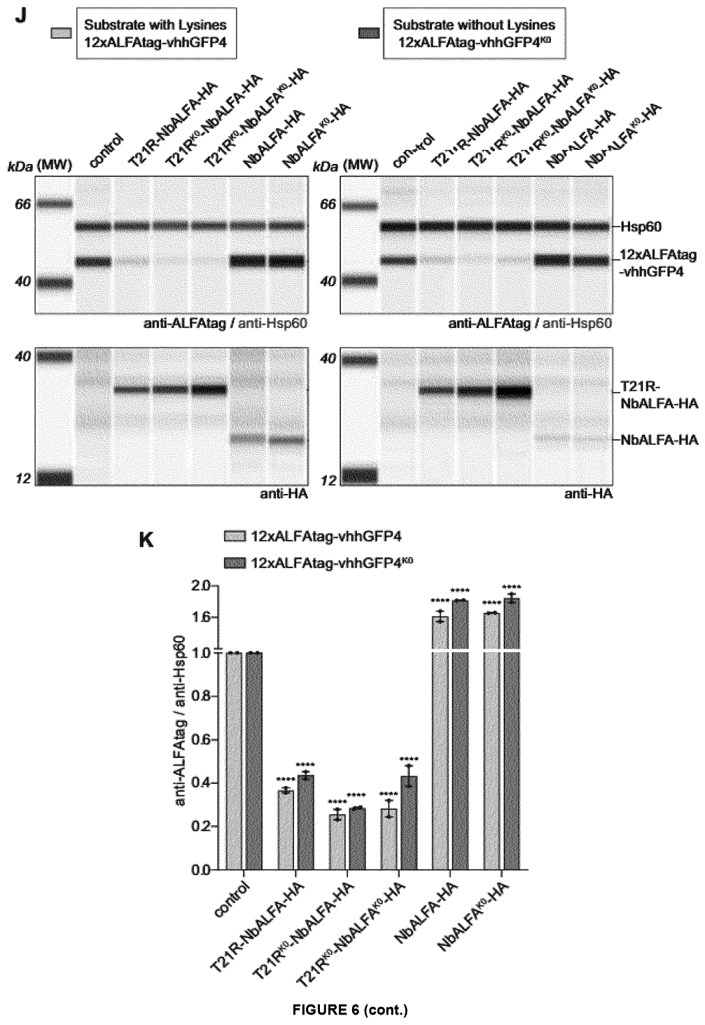

The preceding data show that activated TRIM21 can catalyse simultaneous ubiquitination of itself, antibody and substrate. However, blocking N-terminal TRIM21 ubiquitination does not prevent substrate degradation. To exclude the possibility that ligase lysine autoubiquitination drives substrate degradation, we made a variant of T21R-vhhGFP4 in which all lysines were mutated to arginine (FIG. 6A; T21R^{K0}-vhhGFP4^{K0}). Remarkably, removal of all lysines from T21R-vhhGFP4 had no impact on either the speed or efficiency of substrate degradation (FIGS. 6 B and C). Furthermore, simultaneous blocking the N-terminus via acetylation also had no effect. Taken together, these data show that substrate degradation by TRIM21 is not dependent upon canonical autoubiquitination. Importantly, however, substrate turnover can be uncoupled from ligase turnover as inhibiting N-terminal autoubiquitination reduced ligase depletion without altering substrate degradation (FIG. 6C).

To test whether Trim-Away is driven by substrate lysine ubiquitination, we designed model substrates comprising a dodecameric ALFAtag repeat, which naturally contains no lysine residues (Gotzke et al., 2019), fused to a wild-type or lysineless anti-GFP nanobody (FIG. 6D). These substrates were expressed in either wild-type or TRIM21 knockout (T21 KO) RPE-1 cells, which were then electroporated with ALFAtag nanobody fused to IgG Fc (vhhALFA-Fc). The ALFA-Fc is predicted to bind to the ALFAtag substrate and recruit endogenous TRIM21 via Fc interaction, leading to TRIM21 clustering, activation and degradation (FIG. 6E). Indeed this is what was observed, with significant degradation in wild-type but not T21 KO cells (FIGS. 6F and G). Note that the addition of ALFA-Fc actually increased substrate levels in the absence of TRIM21, suggesting that the nanobody:tag complex is more stable. Importantly, degradation was not dependent upon substrate lysines, as both wild-type and lysiness substrates were equally well degraded (FIG. 6F&G). As expected for a Trim-Away experiment, endogenous TRIM21 was also degraded alongside each substrate (FIG. 6H). As it is formally possible that degradation of a TRIM21:substrate complex requires only one partner to undergo lysine ubiquitination, we modified our assay to remove lysines simultaneously from both ligase and substrate. To do this, we complemented our model substrate with a model ligase comprising a TRIM21 RING fused to the anti-ALFAtag nanobody (T21R-vhhALFA; FIG. 6I). In this assay, the ALFAtag substrate is predicted to recruit multiple T21R-vhhALFA ligases, leading to ligase clustering, activation and degradation (FIG. 6I). As before, removal of all lysines from the substrate had no impact on substrate degradation (FIGS. 6J and K). Strikingly however, Trim-Away was equally efficient when both ligase and substrate were lysineless (FIGS. 6J and K). Taken together, our data show that ligase autoubiquitination does not drive TRIM21-mediated degradation but neither does substrate lysine ubiquitination. This finding may explain the efficiency with which Trim-Away degrades diverse substrates (Clift et al., 2017).

This data also shows that while TRIM21 activation results in ubiquitination of both ligase and substrate, autoubiquitination is regulatory and not required for substrate degradation. Substrate binding stimulates N-terminal RING autoubiquitination by the E2 Ube2W, but when inhibited, for example by N-terminal acetylation, this does not prevent substrate ubiquitination or substrate degradation and has no impact on TRIM21 activity. The inventors have found that uncoupling ligase and substrate degradation prevents ligase recycling, and extends functional persistence in cells.

Therefore, the data also establishes that by blocking the N-terminal of a RING containing protein construct, to inhibit N-terminal RING autoubiquitination it is possible to provide RING containing fusion protein with increased cellular half-life, without substantially effecting substrate degradation.

Although the present disclosure has been described in detail with reference to the specific features, it will be apparent to those skilled in the art that this description is only for a preferred embodiment and does not limit the scope of the present disclosure.

### Materials Used:

TABLE 1

| Cell Line | Source | Identifier | additional information |
|---|---|---|---|
| HEK293T | ATCC; CRL-3216 | CVCL_0063 | Parental cell line |
| HEK293T TRIM21 | Dickson C et al. | n/a | TRIM21 CRISPR knockout |
| KO | 2018; | | |
| hTERT-RPE-1 | ATCC; CRL-4000 | CVCL_4388 | Parental cell line |
| hTERT-RPE-1 | Jingwei Zeng et al | n/a | TRIM21 CRISPR knockout |
| TRIM21 KO | (2021) | | |
| hTERT-RPE-1 | | n/a | Stable cell line (pT21MPP- |
| TRIM21 KO TRIM21- | | | TRIM21-HA) |
| HA | | | |
| hTERT-RPE-1 | | n/a | Stable cell line (pPMEZ-CAV1- |
| CAV1-mEGFP | | | mEGFP) |
| hTERT-RPE-1 | | n/a | Stable cell line (pPMEZ-CAV1- |
| CAV1-mEGFP-Halo | | | mEGFP-Halo) |
| NIH3T3-CAV1-EGFP | Shvets E et al | n/a | TALENs heterozygous EGFP |
| | (2015) | | knockin at 3′ of endogenous |
| | | | caveolin 1 gene |

TABLE 2

| Antibody | Source | Identifier | additional information |
|---|---|---|---|
| humanised anti- | Foss S et al, | Foss S et al, | Used for Adv5 neutralisation and |
| hexon IgG1 9C12 | 2016 | 2016 | signalling assays |
| humanised anti- | Foss S et al, | Foss S et al, | Used for Adv5 neutralisation and |
| hexon IgG1 | 2016 | 2016 | signalling assays |
| 9C12^{H433A} | | | |
| mouse anti-GFP | Rockland; | Abcam Cat # | Used as monoclonal anti-GFP |
| clone 9F9.F9 | 600-301-215 | AB_218216 | antibody for Trim-Away in cells and |
| | | | in vitro. Used as ‘antibody’ for in |
| | | | vitro experiments. |
| mouse anti-GFP | Roche; | AB_390913 | IB: 1:2000 |
| (clones 7.1 and | 11814460001 | | |
| 13.1) | | | |
| NbALFA-msIgG1- | NanoTag; | NanoTag; | Jess 1:20; used as anti-ALFAtag |
| Fc | N1582 | N1582 | antibody for Trim-Away |
| | | | experiments |
| rabbit anti-ERK1 | abcam; | AB_732202 | IB 1:5000; used as anti-ERK1 |
| clone Y72 | ab214168 | | antibody for Trim-Away |
| | | | experiments |
| rabbit anti-IKKα | abcam; | AB_733070 | IB 1:5000; used as anti-IKKα |
| clone Y463 | ab169743 | | antibody for Trim-Away |
| | | | experiments |
| rabbit anti-TRIM21 | Cell Signalling | AB_2800177 | IB 1:2000; recognises PRYSPRY |
| clone D1O1D | Technology; | | domain of TRIM21 (to blot for R-PS |
| | #92043 | | constructs) |
| mouse anti-TRIM21 | Santa Cruz | AB_628286 | IB 1:500 |
| clone D-12 | Biotechnology; | | |
| | sc-25351 | | |
| rabbit anti-vhh | GenScript; | AB_2734123 | IB 1:2000 |
| | A01860 | | |
| rat anti-HA clone | roche; | AB_390917 | IB 1:500; Jess 1:10 |
| 3F10 HRP- | 12013819001 | | |
| conjugated | | | |
| rabbit anti-Vinculin | abcam; | AB_11144129 | IB 1:50,000 |
| clone EPR8185 | ab217171 | | |
| rabbit anti-COXIV | LI-COR; 926- | AB_2783000 | IB 1:5000 |
| | 42214 | | |
| rabbit anti-Hsp60 | bio-techne; | AB_2118931 | Jess 1:00 |
| | AF1800 | | |
| Rabbit anti-Mouse | Dako; P0260 | AB_2636929 | IB 1:5000 |
| IgG HRP- | | | |
| conjugated | | | |
| Goat anti-Mouse | Millipore; | AB_805324 | IB 1:5000 |
| light chain specific, | AP200P | | |
| HRP-conjugated | | | |
| Goat anti-Mouse | LI-COR; 925- | AB_2687825 | IB 1:5000 |
| IgG, IRDye 800CW | 32210 | | |
| Goat anti-Rabbit | Thermo | AB_228338 | IB 1:5000 |
| IgG HRP- | Fisher; #31462 | | |
| conjugated | | | |
| Mouse anti-Rabbit | Millipore; | AB_827270 | IB 1:5000 |
| light chain specific, | MAB201P | | |
| HRP-conjugated | | | |
| Goat anti-Rabbit | Lil-COR; 925- | AB_2721181 | IB 1:5000 |
| lgG, IRDye 680RD | 68071 | | |

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallogr D Biol Crystallogr 66, 213-221. Afonine, P. V., Grosse-Kunstleve, R. W., Echols, N., Headd, J. J., Moriarty, N. W., Mustyakimov, M., Terwilliger, T. C., Urzhumtsev, A., Zwart, P. H., and Adams, P. D. (2012). Towards automated crystallographic structure refinement with phenix.refine. Acta Crystallogr D Biol Crystallogr 68, 352-367.
Aksnes, H., Ree, R., and Arnesen, T. (2019). Co-translational, Post-translational, and Non-catalytic Roles of N-Terminal Acetyltransferases. Mol Cell 73, 1097-1114.
Cadena, C., Ahmad, S., Xavier, A., Willemsen, J., Park, S., Park, J. W., Oh, S. W., Fujita, T., Hou, F., Binder, M., et al. (2019). Ubiquitin-Dependent and -Independent Roles of E3 Ligase RIPLET in Innate Immunity. Cell 177, 1187-1200 e1116.
Clift, D., McEwan, W. A., Labzin, L. I., Konieczny, V., Mogessie, B., James, L. C., and Schuh, M. (2017). A Method for the Acute and Rapid Degradation of Endogenous Proteins. Cell 171, 1692-1706 e1618. Diaz-Griffero, F., Li, X., Javanbakht, H., Song, B., Welikala, S., Stremlau, M., and Sodroski, J. (2006). Rapid turnover and polyubiquitylation of the retroviral restriction factor TRIM5. Virology 349, 300-315.
Diaz-Griffero, F., Qin, X. R., Hayashi, F., Kigawa, T., Finzi, A., Sarnak, Z., Lienlaf, M., Yokoyama, S., and Sodroski, J. (2009). A B-box 2 surface patch important for TRIM5alpha self-association, capsid binding avidity, and retrovirus restriction. J Virol 83, 10737-10751.
Dickson, C., Fletcher, A. J., Vaysburd, M., Yang, J. C., Mallery, D. L., Zeng, J., Johnson, C. M., McLaughlin, S. H., Skehel, M., Maslen, S., et al. (2018). Intracellular antibody signalling is regulated by phosphorylation of the Fc receptor TRIM21. Elife 7 e32660.
Emsley, P., and Cowtan, K. (2004). Coot: model-building tools for molecular graphics. Acta Crystallogr D Biol Crystallogr 60, 2126-2132.
Fletcher, A. J., Mallery, D. L., Watkinson, R. E., Dickson, C. F., and James, L. C. (2015b). Sequential ubiquitination and deubiquitination enzymes synchronize the dual sensor and effector functions of TRIM21. Proc Natl Acad Sci USA 112, 10014-10019.
Fletcher, A. J., Vaysburd, M., Maslen, S., Zeng, J., Skehel, J. M., Towers, G. J., and James, L. C. (2018). Trivalent RING Assembly on Retroviral Capsids Activates TRIM5 Ubiquitination and Innate Immune Signaling. Cell Host Microbe 24, 761-775 e766.
Foss, S., Watkinson, R. E., Grevys, A., McAdam, M. B., Bern, M., Hoydahl, L. S., Dalhus, B., Michaelsen, T. E., Sandlie, I., James, L. C., et al. (2016). TRIM21 Immune Signaling Is More Sensitive to Antibody Affinity Than Its Neutralization Activity. J Immunol 196, 3452-3459.
Galao, R. P., Wilson, H., Schierhorn, K. L., Debeljak, F., Bodmer, B. S., Goldhill, D., Hoenen, T., Wilson, S. J., Swanson, C. M., and Neil, S. J. D. (2022). TRIM25 and ZAP target the Ebola virus ribonucleoprotein complex to mediate interferon-induced restriction. PLoS Pathog 18, e1010530.
Gotzke, H., Kilisch, M., Martinez-Carranza, M., Sograte-Idrissi, S., Rajavel, A., Schlichthaerle, T., Engels, N., Jungmann, R., Stenmark, P., Opazo, F., et al. (2019). The ALFA-tag is a highly versatile tool for nanobody-based bioscience applications. Nat Commun 10, 4403.
Herquel, B., Ouararhni, K., Khetchoumian, K., Ignat, M., Teletin, M., Mark, M., Bechade, G., Van Dorsselaer, A., Sanglier-Cianferani, S., Hamiche, A., et al. (2011). Transcription cofactors TRIM24, TRIM28, and TRIM33 associate to form regulatory complexes that suppress murine hepatocellular carcinoma. Proc Natl Acad Sci USA 108, 8212-8217.
James, L. C., Keeble, A. H., Khan, Z., Rhodes, D. A., and Trowsdale, J. (2007). Structural basis for PRYSPRY-mediated tripartite motif (TRIM) protein function. Proc Natl Acad Sci USA 104, 6200-6205.
Kato, K., Ahmad, S., Zhu, Z., Young, J. M., Mu, X., Park, S., Malik, H. S., and Hur, S. (2021). Structural analysis of RIG-1-like receptors reveals ancient rules of engagement between diverse RNA helicases and TRIM ubiquitin ligases. Mol Cell 81, 599-613 e598.
Kiss, L., Clift, D., Renner, N., Neuhaus, D., and James, L. C. (2021). RING domains act as both substrate and enzyme in a catalytic arrangement to drive self-anchored ubiquitination. Nature Communications 12, 1220.
Kiss, L., and James, L. C. (2022). The molecular mechanisms that drive intracellular neutralization by the antibody-receptor and RING E3 ligase TRIM21. Semin Cell Dev Biol 126, 99-107.
Kiss, L., Zeng, J., Dickson, C. F., Mallery, D. L., Yang, J. C., McLaughlin, S. H., Boland, A., Neuhaus, D., and James, L. C. (2019). A tri-ionic anchor mechanism drives Ube2N-specific recruitment and K63-chain ubiquitination in TRIM ligases. Nat Commun 10, 4502.
Lau, Y. K., Baytshtok, V., Howard, T. A., Fiala, B. M., Johnson, J. M., Carter, L. P., Baker, D., Lima, C. D., and Bahl, C. D. (2018). Discovery and engineering of enhanced SUMO protease enzymes. J Biol Chem 293, 13224-13233.
Liman, E. R., Tytgat, J., and Hess, P. (1992). Subunit stoichiometry of a mammalian K+ channel determined by construction of multimeric cDNAs. Neuron 9, 861-871.
Mallery, D. L., McEwan, W. A., Bidgood, S. R., Towers, G. J., Johnson, C. M., and James, L. C. (2010). Antibodies mediate intracellular immunity through tripartite motif-containing 21 (TRIM21). Proc Natl Acad Sci USA 107, 19985-19990.
McEwan, W. A., Hauler, F., Williams, C. R., Bidgood, S. R., Mallery, D. L., Crowther, R. A., and James, L. C. (2012). Regulation of virus neutralization and the persistent fraction by TRIM21. J Virol 86, 8482-8491.
McEwan, W. A., Tam, J. C., Watkinson, R. E., Bidgood, S. R., Mallery, D. L., and James, L. C. (2013). Intracellular antibody-bound pathogens stimulate immune signaling via the Fc receptor TRIM21. Nat Immunol 14, 327-336.
Pagani, I., Poli, G., and Vicenzi, E. (2021). TRIM22. A Multitasking Antiviral Factor. Cells 10.
Pao, K. C., Wood, N. T., Knebel, A., Rafie, K., Stanley, M., Mabbitt, P. D., Sundaramoorthy, R., Hofmann, K., van Aalten, D. M. F., and Virdee, S. (2018). Activity-based E3 ligase profiling uncovers an E3 ligase with esterification activity. Nature 556, 381-385.
Pickart, C. M., and Raasi, S. (2005). Controlled Synthesis of Polyubiquitin Chains. In Ubiquitin and Protein Degradation, Part B, pp. 21-36.
Robbez-Masson, L., Tie, C. H. C., Conde, L., Tunbak, H., Husovsky, C., Tchasovnikarova, I. A., Timms, R. T., Herrero, J., Lehner, P. J., and Rowe, H. M. (2018). The HUSH complex cooperates with TRIM28 to repress young retrotransposons and new genes. Genome Res 28, 836-845.
Rold, C. J., and Aiken, C. (2008). Proteasomal degradation of TRIM5alpha during retrovirus restriction. PLoS Pathog 4, e1000074.
Sanchez, J. G., Okreglicka, K., Chandrasekaran, V., Welker, J. M., Sundquist, W. I., and Pornillos, O. (2014b). The tripartite motif coiled-coil is an elongated antiparallel hairpin dimer. Proc Natl Acad Sci USA 111, 2494-2499.
Shvets, E., Bitsikas, V., Howard, G., Hansen, C. G., and Nichols, B. J. (2015). Dynamic caveolae exclude bulk membrane proteins and are required for sorting of excess glycosphingolipids. Nat Commun 6, 6867.
Stremlau, M., Owens, C. M., Perron, M. J., Kiessling, M., Autissier, P., and Sodroski, J. (2004). The cytoplasmic body component TRIM5alpha restricts HIV-1 infection in Old World monkeys. Nature 427, 848-853.
Studier, F. W. (2005). Protein production by auto-induction in high density shaking cultures. Protein Expr Purif 41, 207-234.
Towers, G. J. (2007). The control of viral infection by tripartite motif proteins and cyclophilin A. Retrovirology 4, 40.
Tsuchida, T., Zou, J., Saitoh, T., Kumar, H., Abe, T., Matsuura, Y., Kawai, T., and Akira, S. (2010). The ubiquitin ligase TRIM56 regulates innate immune responses to intracellular double-stranded DNA. Immunity 33, 765-776.
Vittal, V., Shi, L., Wenzel, D. M., Scaglione, K. M., Duncan, E. D., Basrur, V., Elenitoba-Johnson, K. S., Baker, D., Paulson, H. L., Brzovic, P. S., et al. (2015). Intrinsic disorder drives N-terminal ubiquitination by Ube2w. Nat Chem Biol 11, 83-89.
Vittal, V., Wenzel, D. M., Brzovic, P. S., and Klevit, R. E. (2013a). Biochemical and structural characterization of the ubiquitin-conjugating enzyme UBE2W reveals the formation of a noncovalent homodimer. Cell Biochem Biophys 67, 103-110.
Vittal, V., Wenzel, D. M., Brzovic, P. S., and Klevit, R. E. (2013b). Biochemical and structural characterization of the ubiquitin-conjugating enzyme UBE2W reveals the formation of a noncovalent homodimer. Cell Biochem Biophys 67, 103-110.
Vranken, W. F., Boucher, W., Stevens, T. J., Fogh, R. H., Pajon, A., Llinas, M., Ulrich, E. L., Markley, J. L., Ionides, J., and Laue, E. D. (2005). The CCPN data model for NMR spectroscopy: development of a software pipeline. Proteins 59, 687-696.
Wagner, J. M., Roganowicz, M. D., Skorupka, K., Alam, S. L., Christensen, D., Doss, G., Wan, Y., Frank, G. A., Ganser-Pornillos, B. K., Sundquist, W. I., et al. (2016). Mechanism of B-box 2 domain-mediated higher-order assembly of the retroviral restriction factor TRIM5alpha. Elife 5.
Weyer, F. A., Gumiero, A., Lapouge, K., Bange, G., Kopp, J., and Sinning, I. (2017). Structural basis of HypK regulating N-terminal acetylation by the NatA complex. Nat Commun 8, 15726.
Zeng, J., Santos, A. F., Mukadam, A. S., Osswald, M., Jacques, D. A., Dickson, C. F., McLaughlin, S. H., Johnson, C. M., Kiss, L., Luptak, J., et al. (2021). Target-induced clustering activates Trim-Away of pathogens and proteins. Nat Struct Mol Biol 28, 278-289.
Zeng, J., Slodkowicz, G., and James, L. C. (2019). Rare missense variants in the human cytosolic antibody receptor preserve antiviral function. Elife 8.
Zhang, J., Hu, M. M., Wang, Y. Y., and Shu, H. B. (2012). TRIM32 protein modulates type I interferon induction and cellular antiviral response by targeting MITA/STING protein for K63-linked ubiquitination. J Biol Chem 287, 28646-28655.
Freemont P S et al (1991) A novel cysteine-rich sequence motif. Cell 64: 483-484.
Meroni G and Roux G, TRIM/RBCC, a novel class of ‘single protein RING finger’ E3 ubiquitin ligases (2005) BioEssays 27, 11:1147-1157.
Marin, I. et al, Origin and diversification of TRIM ubiquitin ligases. PLoS One 7, e50030 (2012).
Atschul S F et al., Basic local alignment search tool, J. Mol. Biol. 215, 403-410 (1990).
Deshaies R J and Joazeiro C, RING Domain E3 Ubiquitin Ligases, Annu. Rev. Biochem (2009) 78:399-434
Trenevska I, Li D and Banham A H (2017) Therapeutic Antibodies against Intracellular Tumor Antigens. Front. Immunol. 8:1001.
Schapira et al. (2019) Targeted protein degradation: expanding the toolbox, Nature Reviews Drug Discovery, 18:949-963).
Castro-Dopico, T., et al. Anti-commensal IgG Drives Intestinal Inflammation and Type 17 Immunity in Ulcerative Colitis (2019). Immunity 50, 1099-1114 e1010.
Chen, X et al. (2019). Degradation of endogenous proteins and generation of a null-like phenotype in zebrafish using Trim-Away technology, Genome Biology 20, 19.

1. A fusion protein comprising:
at least a first RING domain; and
an adaptor domain that is capable of localising the RING domain with a substrate, and
wherein the fusion protein is incapable of N-terminal autoubiquitination.

2. The fusion protein according to claim 1 wherein the fusion protein is:
N-terminally acetylated;
N-terminally methylated; or
comprises a chemical moiety coupled to the N-terminus of the fusion protein, wherein the chemical moiety inhibits ubiquitination of the fusion protein.

3. The fusion protein according to claim 1 wherein the N-terminal of the fusion protein comprises an N-Acetyltransferase recognition site, preferably wherein the N-terminal of the fusion protein comprises the sequence DDDI, or EEEI.

4. The fusion protein according to claim 1 wherein the N-terminal of the fusion protein can undergo N-terminal cyclisation, preferably wherein the fusion protein can undergo N-pyroglutamate cyclisation.

5. The fusion protein according to claim 1 wherein the fusion protein comprises glutamic acid, glutamine or pyroglutamate as the N-terminal residue.

6. The fusion protein according to claim 1 wherein at least the N-terminal amino acid of fusion protein is substituted with an amino acid or amino acid sequence that inhibits the ability of the E2 enzyme Ube2W to ubiquitinate the fusion protein.

7. The fusion protein according to any one of claims 1 to 6 wherein the fusion protein:
comprises a second RING domain, wherein the second RING domain is between the first RING domain and the adaptor domain;
does not comprise a coiled-coil domain and/or a B-box domain; and/or
comprises linker sequences between the RING domains and the adaptor sequence.

8. The fusion protein according to any one of claims 1 to 7 wherein the RING domains are derived from TRIM polypeptides.

9. The fusion protein according to claim 8 wherein the TRIM polypeptide is selected from the group consisting of TRIM5, TRIM7, TRIM19, TRIM21, TRIM25, TRIM28 and TRIM 32, preferably TRIM21.

10. The fusion protein according to any one of claims 1 to 9 wherein the adaptor sequence is:
a protein targeting domain selected from a PRYSPRY domain, an antibody or antibody fragment thereof, or antibody mimetic, wherein the antibody fragment is preferably selected from the group consisting of a Fab, Fab′, F(ab′)2, scFab, Fv, scFV, dAB, VL fragments thereof, VH fragments thereof and V_{HH} fragments thereof; or a.
encodes a protein or fragment thereof capable of locating the RING domain to the substrate.

11. A nucleic acid construct encoding the fusion protein according to any one of claims 1 to 10.

12. A nucleic acid construct comprising a first nucleic acid sequence encoding a first RING domain, and a second nucleic acid sequence encoding an adaptor domain, wherein the nucleic acid construct encodes for a fusion protein that is incapable of autoubiquitination.

13. A nucleic acid construct according to claims 11 or 12, wherein the construct does not encode for a coiled-coil domain; does not encode for or a B-Box domain or does not encode for a coiled-coil domain and a B-box domain.

14. The nucleic acid construct according to any one of claims 11 to 13 in the form of a vector preferably wherein the vector is viral delivery vector, more preferably an adeno-associated virus (AAV) vector.

15. A pharmaceutical composition comprising a fusion protein according to any one of claims 1 to 10 or a nucleic acid according to any one of claims 11 to 14, and a pharmaceutically acceptable carrier and/or excipient.

16. A fusion protein according to anyone of claims 1 to 10 or a nucleic construct according to any one of claims 11 to 14 for use as a medicament.

17. A method of degrading a target protein in a cell comprising introducing a fusion protein of any one of claims 1 to 10 or a nucleic construct according to any one of claims 11 to 14 into the cell.

18. A method of increasing the cellular half-life of a fusion protein comprising a RING domain and an adaptor domain where the adaptor domain is capable of localising the RING domain with a substrate, the method comprising modifying the fusion protein such that it is incapable of N-terminal autoubiquitination.

19. The method according to claim 18 wherein modifying the fusion protein comprises:
incubating the fusion protein with an N-acetyl transferase (NAT) and acetyl-coA to N-terminally acetylate the fusion protein;
methylating the N-terminal amino acid of the fusion protein;
coupling a chemical moiety to the N-terminal amino acid of the fusion protein, wherein the chemical moiety reduces the ability of E2 enzymes Ube2W to ubiquitinate the fusion domain;
introducing an N-Acetyltransferase recognition site, preferably the sequence DDDI, to the N-terminal of the fusion protein, optionally and incubating the fusion protein with an N-acetyl transferase (NAT) and acetyl-coA to N-terminally acetylate the fusion protein;
modifying the fusion protein so that the fusion protein can undergo N-terminal cyclization;
modifying the fusion protein so that the fusion protein can undergo N-terminal pyroglutamate cyclisation;
introducing a glutamic acid or a glutamine residue to the N-terminal of the fusion protein, and optionally incubating the fusion protein with a glutaminyl cyclase; or
substituting at least the N-terminal amino acid of the RING domain with an amino acid or amino acid sequence that inhibits the ability of the E2 enzymes Ube2W to ubiquitinate the fusion protein.

20. A method of producing the fusion protein of any one of claims 1 to 10, comprising;
(a) culturing a host cell comprising a first vector encoding for a fusion protein comprising a RING domain and an adaptor domain, under conditions to permit expression of the fusion protein; and
(b) obtaining the expressed protein from the host cell, wherein the fusion protein is incapable of N-terminal autoubiquitination.

21. The method according to claim 20 wherein the first vector also encodes a glutaminyl cyclase and the fusion protein expressed comprises an N-terminal glutamic acid or an N-terminal glutamine, and the method comprises culturing the host cell under conditions to permit expression of the fusion protein and the glutaminyl cyclase.
